(19) United States
(12) Patent Application Publication
(10) Pub. No.: US 2025/0357010 A1
(43) Pub. Date: Nov. 20, 2025

(54) SYSTEMS AND METHODS FOR MONITORING PRESCRIPTION ORDERING PATTERNS

(71) Applicant: DECISION RX INC., Indianapolis, IN (US)

(72) Inventors: Bradley A. Bostic, Indianapolis, IN (US); Charles J. Clarke, Indianapolis, IN (US); Ryan C. Kennedy, Carmel, IN (US); Peter J. Plantes, Indianapolis, IN (US); Charles David Girard, JR., Indianapolis, IN (US)

(73) Assignee: DECISION RX INC., Indianapolis, IN (US)

(21) Appl. No.: 19/289,596

(22) Filed: Aug. 4, 2025

Related U.S. Application Data
(63) Continuation of application No. 17/205,126, filed on Mar. 18, 2021.
Application No. 17/205,126 is a continuation of application No. 17/023,516, filed on Sep. 17, 2020, now abandoned.
Application No. 17/023,516 is a continuation-in-part of application No. 16/825,396, filed on Mar. 20, 2020, now abandoned.
Application No. 16/825,396 is a continuation-in-part of application No. 16/778,377, filed on Jan. 31, 2020, now abandoned.
Application No. 16/778,377 is a continuation-in-part of application No. 16/535,863, filed on Aug. 8, 2019, now abandoned.
(60) Provisional application No. 62/716,090, filed on Aug. 8, 2018.
(60) Provisional application No. 62/800,086, filed on Feb. 1, 2019.

(51) Int. Cl. G16H 50/30; G16H 15/00; G16H 50/80

(52) U.S. Cl. CPC G16H 50/30; G16H 15/00; G16H 50/80

(57) ABSTRACT

Systems and methods are provided for characterizing the activities of one or more patients in a health care system using an interception module for retrieving prescription drug data relating to the one or more patients, a correlation module that ensures that the prescription drug data is associated with the correct records of the one or more patients, and an analytics module that determines whether prescription ordering patterns for the one or more patients and indicates whether a subset of the ordering patterns is anomalous as compared with a stored ordering criterion.

### CROSS-REFERENCE TO RELATED APPLICATIONS

This application is a continuation of U.S. application Ser. No. 17/205,126 filed on Mar. 18, 2021, entitled Systems and Methods for Monitoring Prescription Ordering Patterns, which is a continuation of U.S. application Ser. No. 17/023,516 filed on Sep. 17, 2020, entitled Methods and Systems for a Health Monitoring Command Center and Workforce Advisor, now abandoned, which is a continuation-in-part of U.S. application Ser. No. 16/825,396 filed on Mar. 20, 2020, entitled Methods and Systems for a Pharmacological Tracking and Representation of Health Attributes using Digital Twin, now abandoned, which is a continuation-in-part of U.S. application Ser. No. 16/778,377 filed on Jan. 31, 2020, entitled Methods and Systems for a Pharmacological Tracking and Reporting Platform, now abandoned, which (i) claims priority to U.S. provisional application No. 62/800,086 filed on Feb. 1, 2019, and (ii) is a continuation-in-part of U.S. application Ser. No. 16/535,863 filed on Aug. 8, 2019, entitled Methods and Systems for a Pharmacological Tracking and Reporting Platform, now abandoned, which claims priority to U.S. provisional application No. 62/716,090 filed on Aug. 8, 2018. Each of the above applications is hereby incorporated by reference as if fully set forth herein in its entirety.

### FIELD

The present disclosure relates to a pharmacological tracking platform facilitating representation of health attributes using one or more digital twins.

### BACKGROUND

It is estimated that approximately 80% of Americans are prescribed at least one pharmaceutical drug. Many people who are prescribed pharmaceutical drugs, however, may be prescribed the wrong drug, which can lead to adverse reactions, ineffective treatment, or even death. In some scenarios, a patient may be taking two medications that are not compatible with one another. In other scenarios, the patient may be physiologically unable to metabolize or otherwise process one of the active ingredients in the medication. These conditions may be averted if the patient is prescribed appropriate tests prior to being prescribed a treatment.

Moreover, many patients that are prescribed medications are misusing their drugs. In some scenarios, patients may be abusing the medication they are prescribed (e.g., opiates, amphetamines, and/or benzodiazepines). In other scenarios, patients may be using the drug with an incompatible over the counter medication or may be using the medication improperly (e.g., taking the medication too infrequently or without following the instructions). In other cases, prescribed medications may be diverted for use by individual's other than the one for whom the medication is prescribed, such as for sale on the black market or for unprescribed use by friends or family members. In any of these scenarios, a patient's health may be adversely affected and/or the costs of treating the patient may increase due to the improper use of the medication.

Applicant appreciates that a need exists for improved methods and systems for detecting and addressing situations involving improper prescription of medication, improper utilization of prescribed medications, and diversion of prescribed medications to unprescribed uses. Applicant also appreciates that a need exists for improved simulation of patient medical and diagnostic states and improvements to those states based on presented contingencies and options in care and health of the patient.

### SUMMARY

Improved methods, systems, components, processes, modules, and other elements (collectively referred to alternatively herein as the “pharmacological tracking platform,” or simply as the “platform”) for detecting and addressing situations involving improper prescription of medication, improper utilization of prescribed medications, and diversion of prescribed medications to unprescribed uses.

According to some embodiments of the present disclosure, a method for determining a patient health state, includes ingesting healthcare data of a patient received from one of a plurality of patient data providers; enriching at least one new data element of the ingested healthcare data based on the determined one or more relationships among the ingested healthcare data; transmitting the at least one new data element to a raw data cluster; transmitting the raw data cluster to a machine learning module; and using the machine learning module to compute a current health state for the patient based at least in part on modeling the at least one enriched data element and the ingested healthcare data.

In embodiments, the method includes storing the determined one or more relationships in a data store. In embodiments, the data store is further configured to store lifestyle and wellness records of the patient, the lifestyle and wellness information including information related to one or more of diet, smoking, alcohol consumption, and exercise habits.

In embodiments, the healthcare data may derive from an electronic medical record. In embodiments, the healthcare data derives from a physician's database. In embodiments, the machine learning module is configured to train a machine learned model that is leveraged by a test management system. In embodiments, the machine learning module is configured to train a machine learned model that is leveraged by a prescription monitoring system. In embodiments, the machine learning module is configured to train a machine learned neural network model. In embodiments, the machine learned neural network model is a recurrent neural network model. In embodiments, the machine learning module is configured to train a Bayesian model. In embodiments, the machine learning module is configured to train an artificial intelligence system. In embodiments, the machine learning module is configured to train a rules-based recommendation system.

In embodiments, a method for simulating a patient wellness state, includes ingesting, by a computing device, patient data of a patient received from one of a plurality of patient data providers; determining, by the computing device, one or more relationships between the ingested patient data and previously ingested patient data, wherein at least one new enriched data element is created based on the determined one or more relationships; transmitting the at least one new data element to a raw data cluster; storing the determined one or more relationships in a data store, wherein the data store is further configured to store lifestyle and wellness records of the patient, the lifestyle and wellness information including information related to one or more of diet, smoking, alcohol consumption, and exercise habits; transmitting the data store to a machine learning module; and using the machine learning module to simulate a future wellness state of the patient.

In embodiments, the future wellness state is a predicted illness. In embodiments, the machine learning simulation includes pharmaceutical data to simulate the future wellness state contingent upon the patient taking a stated medication. In embodiments, the machine learning simulation includes treatment plan data to simulate the future wellness state contingent upon the patient receiving a stated treatment. In embodiments, the machine learning simulation uses a digital twin of the patient. In embodiments, the digital twin of the patient is matched to a digital twin representing a population of patients sharing a patient health attribute. In embodiments, the digital twin of the patient is matched to a plurality of digital twins, each representing a population of patients receiving a stated treatment, wherein each stated treatment is indicated for the patient. In embodiments, the digital twin of the patient is matched to a plurality of digital twins, each representing a population of patients receiving a stated medication, wherein each stated medication is indicated for the patient. In embodiments, the machine learning simulation uses a plurality of digital twins of the patient.

In embodiments, a method for determining a patient wellness state, includes ingesting healthcare data of a patient received from one of a plurality of patient data providers; enriching at least one new data element of the ingested healthcare data based on the determined one or more relationships among the ingested healthcare data; transmitting the at least one new data element to a raw data cluster; transmitting the raw data cluster to a machine learning module; and using the machine learning module to compute a current health state for the patient based at least in part on modeling the at least one enriched data element and the ingested healthcare data; and classifying the patient within a population of patients, wherein said population of patients is determined based on one or more of lifestyle, diagnosis and/or prognosis, and present or previous healthcare treatments as categorized using the machine learning module.

In embodiments, the healthcare data derives from an electronic medical record. In embodiments, the healthcare data derives from a pharmacy database. In embodiments, the healthcare data derives from a laboratory database. In embodiments, the healthcare data derives from an insurer database. In embodiments, the healthcare data derives from a physician's database. In embodiments, the machine learning module is configured to train a machine learned model that is leveraged by a test management system. In embodiments, the machine learning module is configured to train a machine learned model that is leveraged by a prescription monitoring system.

In embodiments, the machine learning module is configured to train a machine learned neural network model. In embodiments, the machine learned neural network model is a recurrent neural network model.

In embodiments, the machine learning module is configured to train a Bayesian model. In embodiments, the machine learning module is configured to train an artificial intelligence system. In embodiments, the machine learning module is configured to train a rules-based recommendation system. In embodiments, the classification of the patient is according to a conformance to a prescription medication regimen.

In embodiments, a method for configuring classified patient wellness states, includes ingesting, by a computing device, patient data of a patient received from one of a plurality of patient data providers; determining, by the computing device, one or more relationships between the ingested patient data and previously ingested patient data, wherein at least one new enriched data element is created based on the determined one or more relationships; transmitting the at least one new data element to a raw data cluster; storing the determined one or more relationships in a data store, wherein the data store is further configured to store lifestyle and wellness records of the patient, the lifestyle and wellness information including information related to one or more of diet, smoking, alcohol consumption, and exercise habits; transmitting the data store to a machine learning module; using the machine learning module to compute a current health state for the patient; classifying the patient data within a population of patients, wherein said population of patients is determined based on one or more of lifestyle, diagnosis and/or prognosis, and present or previous healthcare treatments as categorized using the machine learning module; and configuring the patient data classification data to transmit to a healthcare provider.

In embodiments, the classification of the patient is according to an International Classification of Diseases (ICD) coding. In embodiments, the classification of the patient is according to a classification criterion specified by the healthcare provider. In embodiments, the classification of the patient is further associated with a confidence score indicating the degree of confidence in the classification. In embodiments, the classification of the patient is a ranked plurality of classifications corresponding to a plurality of populations of patients. In embodiments, the configuration of the patient classification data is based on a stored data transmission rule that is associated with the healthcare provider.

In embodiments, the method includes a method for predicting a future health state, that includes ingesting healthcare data of a patient received from one of a plurality of patient data providers; transmitting the ingested healthcare data to a data store; transmitting the data store to a machine learning module, wherein the machine learning module applies at least one algorithm selected from the set comprising transformation algorithms, normalization operations, and refinement operations; and using the machine learning module to predict a needed future treatment for the patient based on a predicted future health state for the patient.

In embodiments, the healthcare data derives from an electronic medical record. In embodiments, the healthcare data derives from a pharmacy database. In embodiments, the healthcare data derives from a laboratory database. In embodiments, the healthcare data derives from an insurer database. In embodiments, the healthcare data derives from a physician's database.

In embodiments, the machine learning module is configured to train a machine learned model that is leveraged by a test management system.

In embodiments, the machine learning module is configured to train a machine learned model that is leveraged by a prescription monitoring system. In embodiments, the machine learning module is configured to train a machine learned neural network model. In embodiments, the machine learned neural network model is a recurrent neural network model. In embodiments, the machine learning module is configured to train a Bayesian model. In embodiments, the machine learning module is configured to train an artificial intelligence system. In embodiments, the machine learning module is configured to train a rules-based recommendation system.

In embodiments, the rules-based recommendation system includes rules for determining the appropriateness of a treatment. In embodiments, the treatment is a prescription medication. In embodiments, the configuration of the machine learning module to train a rules-based recommendation system includes using training data from a prescription medication data set. In embodiments, the configuration of the machine learning module to train a rules-based recommendation system includes using training data from a prescription medication data set. In embodiments, the configuration of the machine learning module to train a rules-based recommendation system includes using training data from a patient outcomes data set.

In embodiments, a method for determining a medical service need, includes ingesting, by a computing device, patient data of a patient received from one of a plurality of patient data providers; transmitting the ingested data to a data store; transmitting the data store to a machine learning module, wherein the machine learning module wherein applies at least one algorithm selected from the set comprising transformation algorithms, normalization operations, and refinement operations; and using the machine learning module to simulate a future health state for the patient; matching the simulated future health state to a predicted patient medical service need; matching the predicted patient medical service need to at least one of the patient's healthcare providers; and transmitting an alert to the at least one healthcare provider indicating the predicted patient medical service need.

In embodiments, the machine learning simulation uses a digital twin of the patient. In embodiments, the method includes a computerized method for patient digital twin management, that includes receiving health information from a plurality of healthcare communication sources, the health information including data related to an individual patient and data related to a first population of patients and a second population of patients; forming a digital twin of said individual patient based on the health information related to said individual patient, wherein the digital twin of said individual patient is a digital representation of at least one health state of said individual patient; forming digital twins of said first and second populations of patients based on the health information related to at least one of said first and second population of patients, wherein the digital twins are a digital representation of at least one health attribute of at least one of said first and second population of patients; and presenting the digital twin of said individual patient and the digital twin of at least one of said first population of patients and second population of patients.

In embodiments, the method includes receiving healthcare research information derived from a plurality of healthcare research sources; determining, using a machine learning module, whether at least a portion of the healthcare research information is relevant to at least one of said individual patient, said first population of patients, and said second population of patients; and presenting the healthcare research information determined to be relevant to at least one of said individual patient, said first population of patients, and said second population of patients.

In embodiments, the method includes outputting the digital twin of said patient and the digital twin of said population of patients to a machine learning module of the healthcare data system; simulating a future health state of said first population of patients based on the digital twin of said patient using the digital twin of said patient and the machine learning module; simulating a future health state of said second population of patients based on the digital twin of said population of patients via the digital twin of said population of patients and the machine learning module; updating the digital twin of said patient based on the simulation of the future health state of said patient; updating the digital twin of said population of patients based on the simulation of the future health state of said population of patients; and presenting the healthcare research information determined to be relevant to at least one of said individual patient, said first population of patients, and said second population of patients.

In embodiments, simulation of the future health state of said first population of patients and/or the future health state of said second population of patients is performed according to simulation instructions received from one or more healthcare worker. In embodiments, wherein simulation of the future health state of said first population of patients and/or the future health state of said second population of patients is performed according to simulation instructions formed by the machine learning module.

In embodiments, the method includes forming, using a machine learning module, one or more models based on the health information related to at least one of a first and a second population of patients of said population of patients, wherein the one or models are configured to facilitate anticipating one or more responses to medical treatment by at least one of said first population of patients and said second population of patients.

In embodiments, the method includes facilitating opting into one or more treatment programs by at least one of said individual patient, a patient from said first population of patients, and a patient from said second population of patients.

In embodiments, the method includes simulating, using a machine learning module, effects of at least one of one or more drugs and treatment options on at least one of said individual patient, said first population of patients, and said second population of patients.

In embodiments, the method includes comparing simulations of one of one or more drugs and said treatment options to one or more of said treatment programs opted into by at least one of said individual patient, a patient from said first population of patients, and a patient from said second population of patients.

In embodiments, the method includes receiving healthcare study information including at least one of methodology and results of one or more healthcare studies; and comparing, using a machine learning module, the healthcare study information to the simulations of one or more said drugs and said treatment options to determine at least one of reliability and consistency of the simulations of one or more said drugs and treatment options.

In embodiments, a method for patient digital twin management, includes receiving health information from a plurality of healthcare communication sources, the health information including data related to a plurality of patients and data related to a first population of patients and a second population of patients; forming a digital twin of each of said plurality of patients based on the health information related to said plurality of patients, wherein the digital twin of each of said plurality of patients is a digital representation of at least one health state of said plurality of patients; forming digital twins of said first and second populations of patients based on the health information related to at least one of said first and second population of patients, wherein the digital twins are a digital representation of at least one health attribute of at least one of said first and second population of patients; and inferring a patient health state, using a machine learning module, based on a degree of correspondence among at least one of the digital twins based on the plurality of patients and at least one digital twin of said first and second population of patients.

In embodiments, the patient health state inference is based at least in part on a set of patient test data comprising a machine learning module for analyzing a set of at least one of laboratory testing data including at least one corresponding outcome, a correlation module for correlating the outcome with signals from the patient test data, analyzing the testing data corresponding to a set of patients, and providing a listing of a set of patients most likely to have a specified pathology.

In embodiments, the patient health state is a future health state. In embodiments, the patient health state is compared to ideal disease state data, a measure of correspondence between the patent health state and the ideal disease state data is calculated. In embodiments, the ideal disease state data is based upon one or more clinical standards and/or optimal health outcomes. In embodiments, the patient health state is an organ-specific health condition metric. In embodiments, the patient health state is a weighted metric summarizing a plurality of organ-specific health condition metrics.

In embodiments, providing the listing of the set of patients most likely to have the specified pathology includes a listing of a potential gap in current care of each of the patients. In embodiments, the potential gap in current care of the patients is a currently unused, but indicated, medication. In embodiments, providing the listing of the set of patients most likely to have the specified pathology includes a listing of a recommended treatment option for each of the patients. In embodiments, providing the listing of the set of patients most likely to have the specified pathology includes a listing of a recommended lab test for each of the patients.

In embodiments, the method includes predicting an insurance-related event, that includes ingesting healthcare data of a patient received from one of a plurality of patient data providers and physician data relating to the patient from insurance records; enriching at least one new data element of the ingested healthcare and physician data based on the determined one or more relationships among the ingested healthcare and physician data; transmitting the at least one new data element to a machine learning module; and using the machine learning module to predict a future insurance-related event relating to the patient.

In embodiments, the healthcare data derives from an electronic medical record. In embodiments, the healthcare data derives from a pharmacy database. In embodiments, the healthcare data derives from a laboratory database. In embodiments, the healthcare data derives from an insurer database. In embodiments, the healthcare data derives from a physician's database.

In embodiments, the machine learning module is configured to train a machine learned model that is leveraged by a test management system. In embodiments, the machine learning module is configured to train a machine learned model that is leveraged by a prescription monitoring system. In embodiments, the machine learning module is configured to train a machine learned neural network model. In embodiments, the machine learned neural network model is a recurrent neural network model. In embodiments, the machine learning module is configured to train a Bayesian model. In embodiments, the machine learning module is configured to train an artificial intelligence system. In embodiments, the machine learning module is configured to train a rules-based recommendation system. In embodiments, the rules-based recommendation system includes rules for determining the appropriateness of a treatment. In embodiments, the treatment is a prescription medication.

In embodiments, the configuration of the machine learning module to train a rules-based recommendation system includes using training data from a prescription medication data set. In embodiments, the configuration of the machine learning module to train a rules-based recommendation system includes using training data from a prescription medication data set. In embodiments, the future insurance-related event is a reimbursement event. In embodiments, the reimbursement event is a reimbursement denial.

In embodiments, the method includes monitoring insurance billing events, that includes ingesting patient data received from one of a plurality of patient data providers and healthcare services data relating to the patient data; ingesting data relating to insurance reimbursement criteria and insurance reimbursement records relating to the healthcare services data; determining one or more relationships between the ingested patient data, healthcare services data, insurance reimbursement criteria and insurance reimbursement records, and data and previously ingested patient data, healthcare services data, insurance reimbursement criteria and insurance reimbursement records wherein at least one new enriched data set is created based on the determined one or more relationships; transmitting the enriched data set to an analytic engine; using the analytic engine to calculate an insurance reimbursement score, wherein the insurance reimbursement score is based at least in part on an association between the healthcare services data and insurance reimbursement records; and using the analytic engine to calculate an insurance reimbursement score for a future planned health service event based at least in part on a comparison to the plurality of calculated insurance reimbursement scores.

In embodiments, a method for determining a patient wellness state, includes ingesting healthcare data of a patient received from one of a plurality of patient data providers and physician data relating to the patient from insurance records; enriching a data set by computing one or more relationships between the ingested healthcare data and the physician data and previously ingested healthcare data and physician data, wherein at least one new enriched data element is created based on the determined one or more relationships; transmitting the enriched data set to a machine learning module; and using the machine learning module to identify at least one potential medical coding inconsistency among the enriched data set.

In embodiments, the healthcare data derives from an electronic medical record. In embodiments, the healthcare data derives from a pharmacy database. In embodiments, the healthcare data derives from a laboratory database. In embodiments, the healthcare data derives from an insurer database. In embodiments, the healthcare data derives from a physician's database.

In embodiments, the machine learning module is configured to train a machine learned model that is leveraged by a test management system. In embodiments, the machine learning module is configured to train a machine learned model that is leveraged by a prescription monitoring system. In embodiments, the machine learning module is configured to train a machine learned neural network model. In embodiments, the machine learned neural network model is a recurrent neural network model. In embodiments, the machine learning module is configured to train a Bayesian model. In embodiments, machine learning module is configured to train an artificial intelligence system. In embodiments, the machine learning module is configured to train a rules-based recommendation system. In embodiments, the rules-based recommendation system includes rules for determining the appropriateness of a treatment. In embodiments, the treatment is a prescription medication.

In embodiments, the configuration of the machine learning module to train a rules-based recommendation system includes using training data from a prescription medication data set. In embodiments, the configuration of the machine learning module to train a rules-based recommendation system includes using training data from a prescription medication data set. In embodiments, the at least one potential medical coding inconsistency relates to inconsistency between a patent prescription coding and an identified patient metabolite. In embodiments, the inconsistency between the patient prescription coding and the identified patient metabolite is based on the absence of an expected metabolite associated with a medication identified within the patient prescription coding.

In embodiments, the method includes a machine learning device in communication with a healthcare database and configured to receive demographic records, diagnosis records, prescription records, and testing records from a plurality of healthcare databases, from a plurality of healthcare providers, wherein the machine learning device is configured to train an artificial intelligence module based on the demographic records, the diagnosis records, the prescription records, and the testing records; training the artificial intelligence module to identify inconsistencies in the names and/or codes used by the healthcare providers; normalizing the names and/or codes used to identify the tests by the healthcare providers; identifying similar tests used by the healthcare providers based at least in part on the normalized the names and/or codes; and associating each of the identified similar tests with a corresponding code used by at least one insurer; and storing the association.

In embodiments, a method for determining drug dispensing consistency, includes ingesting patient data from at least one patient data provider, wherein the patient data includes at least one of drug toxicology data, metabolite data, patient-reported symptoms, and patient prescriptions; enriching a data set by computing one or more relationships between the ingested patient data and previously ingested patient population data that includes at least one of drug toxicology data, metabolite data, patient-reported symptoms, and patient prescriptions, wherein at least one new enriched data element is created based on the determined one or more relationships; transmitting the enriched data set to a machine learning module; and using a machine learning module to analyze the enriched data set to determine if a patient metabolite reported in a toxicology test is consistent with a known patient prescription.

In embodiments, the healthcare data derives from an electronic medical record. In embodiments, the healthcare data derives from a pharmacy database. In embodiments, the healthcare data derives from a laboratory database. In embodiments, the healthcare data derives from an insurer database. In embodiments, the healthcare data derives from a physician's database.

In embodiments, the machine learning module is configured to train a machine learned model that is leveraged by a test management system. In embodiments, the machine learning module is configured to train a machine learned model that is leveraged by a prescription monitoring system. In embodiments, the machine learning module is configured to train a machine learned neural network model. In embodiments, the machine learned neural network model is a recurrent neural network model. In embodiments, the machine learning module is configured to train a Bayesian model. In embodiments, the machine learning module is configured to train an artificial intelligence system. In embodiments, the machine learning module is configured to train a rules-based recommendation system.

In embodiments, the rules-based recommendation system includes rules for determining the appropriateness of a treatment. In embodiments, the treatment is a prescription medication. In embodiments, the configuration of the machine learning module to train a rules-based recommendation system includes using training data from a prescription medication data set. In embodiments, the configuration of the machine learning module to train a rules-based recommendation system includes using training data from a prescription medication data set. In embodiments, the configuration of the machine learning module to train a rules-based recommendation system includes using training data from a prescription medication metabolization data set. In embodiments, the prescription medication metabolization data set includes time-series data on prescription drug metabolism over a specified time period.

In embodiments, a method for determining drug dispensing consistency, includes ingesting, by a computing device, patient data from at least one patient data provider, wherein the patient data includes at least one of drug toxicology data, metabolite data, patient-reported symptoms, and patient prescriptions; determining, by the computing device, one or more relationships between the ingested patient data and previously ingested patient population data that includes at least one of drug toxicology data, metabolite data, patient-reported symptoms, and patient prescriptions, wherein at least one new enriched data element is created based on the determined one or more relationships; transmitting the at least one new data element to a raw data cluster; storing the determined one or more relationships in a data store; using a machine learning module to analyze data within the data store to determine the if detected metabolites are indicative of a potential adverse patient reaction; and transmitting an alert to the at least one healthcare provider indicating the predicted potential adverse patient reaction.

In embodiments, a method for identifying patient risk, includes ingesting patient data relating to a plurality of patients received from at least one of a plurality of patient data providers; importing the ingested data into at least one data matrix, determining one or more relationships between the ingested patient data relating to a plurality of patients and previously ingested patient data, wherein at least one new enriched data set is created based on the determined one or more relationships; importing the enriched data set into a machine learning module; performing data mining on the enriched data set, using the machine learning module, to determine if a patient within the plurality of patients has a risk of developing a specified clinical indication.

In embodiments, the machine learning module is configured to train an artificial intelligence system. In embodiments, the machine learning module is configured to train a rules-based recommendation system. In embodiments, the rules-based recommendation system includes rules for determining the appropriateness of a treatment. In embodiments, the treatment is a prescription medication. In embodiments, the configuration of the machine learning module to train a rules-based recommendation system includes using training data from a prescription medication data set. In embodiments, the configuration of the machine learning module to train a rules-based recommendation system includes using training data from a prescription medication data set. In embodiments, the risk of developing a specified clinical indication is presented with a confidence level associated with the risk determination.

In embodiments, a method for simulating patient risk, includes ingesting patient data relating to a plurality of patients received from at least one of a plurality of patient data providers; importing the ingested data into at least one data matrix, determining one or more relationships between the ingested patient data relating to a plurality of patients and previously ingested patient data, wherein at least one new enriched data set is created based on the determined one or more relationships; importing the enriched data set into a machine learning module; receiving simulation instructions, wherein the simulation instructions are indicative of one or more drug treatment plans; using the machine learning module to simulate the one or more drug treatment plans; and evaluating the efficacy of the one or more simulated drug treatment plans.

In embodiments, the method includes receiving simulation instructions from a healthcare provider, the simulation instructions being indicative of one or more treatment plans; simulating the one or more treatment plans, application of best clinical practices for a desired clinical outcome, and identification of any gaps in care on said patient via the digital twin of said patient; and evaluating efficacy of the one or more treatment plans.

In embodiments, the method includes simulating application of best clinical practices for a desired clinical outcome on said patient via the digital twin of said patient. In embodiments, the method includes identifying any gaps in care provided to said patient using the digital twin of said patient. In embodiments, the one or more gaps in care include failure by one or more healthcare professionals to follow one or more established clinical standards of care in treating said patient.

In embodiments, the method includes determining one or more prognostication methods suitable for said population of patients using the machine learning module; simulating the one or more prognostication methods on said population of patient via the digital twin of said population of patients; and evaluating efficacy of the one or more prognostication methods.

In embodiments, the method includes receiving simulation instructions from a healthcare researcher, the simulation instructions including one or more research experiments; simulating the one or more research experiments, and results of best clinical practices on at least one of said patient and said population of patients using at least one of the digital twin of said patient and the digital twin of said population of patients.

In embodiments, the method includes receiving simulation instructions from a healthcare researcher, the simulation instructions including one or more drug treatment regimens; simulating the one or more drug treatment regimens on one or both of said patient and said population of patients vs. using at least one of the digital twin of said patient and the digital twin of said population of patients.

In embodiments, the method includes receiving sensor data from one or more Internet of Things (IoT) sensors related to one or both of said patient and said population of patients; and updating at least one of the digital twin of said patient and the digital twin of said population of patients based on said population of patients based on the sensor data.

In embodiments, the method includes outputting the digital twin of said patient and the digital twin of said population of patients to a machine learning module of the healthcare data system; simulating a future health state of said first population of patients based on the digital twin of said patient via the digital twin of said patient and the machine learning module; simulating a future health state of said second population of patients based on the digital twin of said population of patients via the digital twin of said population of patients and the machine learning module; updating the digital twin of said patient based on the simulation of the future health state of said patient; updating the digital twin of said population of patients based on the simulation of the future health state of said population of patients; and presenting to a healthcare worker healthcare research information determined to be relevant to at least one of said individual patient, said first population of patients, and said second population of patients.

In embodiments, simulation of the future health state of said first population of patients and/or the future health state of said second population of patients is performed according to simulation instructions received from one or more of said healthcare workers.

In embodiments, simulation of the future health state of said first population of patients and/or the future health state of said second population of patients is performed according to simulation instructions formed by the machine learning module.

In embodiments, the method includes identifying patient overdose risk, that includes ingesting patient data from at least one patient data provider, wherein the patient data includes at least one of drug toxicology data, metabolite data, patient-reported symptoms, patient prescriptions, and patient adverse events; importing the ingested data into at least one data matrix, determining one or more relationships between the ingested patient data and previously ingested patient population data that includes at least one of drug toxicology data, metabolite data, patient-reported symptoms, patient prescriptions and patient adverse events, wherein at least one new enriched data set is created based on the determined one or more relationships; importing the enriched data set into a machine learning module; using the machine learning module to analyze data within the enriched data set to compute at least one patient drug profile based on the ingested patient data and at least one patient population drug profile based on the ingested patient population data; and calculating an overdose risk score for a patient within the ingested patient data, wherein the overdose risk score is based at least in part on an association between the at least one patient drug profile and the at least one patient population drug profile.

In embodiments, the patient data derives from an electronic medical record. In embodiments, the patient data derives from a pharmacy database. In embodiments, the patient data derives from a laboratory database. In embodiments, the patient data derives from an insurer database. In embodiments, the patient data derives from a physician's database.

In embodiments, the machine learning module is configured to train a machine learned model that is leveraged by a test management system. In embodiments, the machine learning module is configured to train a machine learned model that is leveraged by a prescription monitoring system. In embodiments, the machine learning module is configured to train a machine learned neural network model. In embodiments, the machine learned neural network model is a recurrent neural network model. In embodiments, the machine learning module is configured to train a Bayesian model. In embodiments, the machine learning module is configured to train an artificial intelligence system. In embodiments, the machine learning module is configured to train a rules-based recommendation system.

In embodiments, the rules-based recommendation system includes rules for determining the appropriateness of a treatment. In embodiments, the treatment is a prescription medication. In embodiments, the configuration of the machine learning module to train a rules-based recommendation system includes using training data from a prescription medication data set. In embodiments, the configuration of the machine learning module to train a rules-based recommendation system includes using training data from a prescription medication data set.

In embodiments, a system for characterizing the activities of an individual physician in a health care drug prescription system, includes an interaction module identifying each sales and service representative with whom a physician has interacted, the interaction module creating a physician interaction dataset; an ordering module identifying each organization from whom the physician orders prescription drugs, the ordering module creating a physician ordering dataset; a prescription tracking module identifying each of the physician's prescriptions fulfilled, the prescription tracking module creating a prescription fulfillment dataset; a data ingestion module for retrieving the physician interaction dataset, the physician ordering dataset, and the prescription fulfillment dataset, and importing each of said datasets to a prescription drug monitoring program dataset, wherein the prescription drug monitoring program dataset identifies the plurality of relationships between each physician in the physician interaction dataset, physician ordering dataset, prescription fulfillment dataset; and a machine learning module to identify at least one prescription fulfillment within the prescription drug monitoring program dataset that does not conform to a specified prescription rule.

In embodiments, the identification of at least one prescription fulfillment within the prescription drug monitoring program dataset that does not conform to a specified prescription rule generates an alert to a healthcare provider. In embodiments, the specified prescription rule is a plurality of specified prescription rules.

In embodiments, a system for relationship discovery of physician prescription behavior, includes ingesting patient data received from a plurality of healthcare data providers and physician data from a plurality of physician practices; and determining one or more relationships between the ingested patient data and physician data, wherein at least one new enriched data set is created based on the determined one or more relationships, wherein a confidence level associated with at least one of the determined relationships is assigned based at least in part on patient data being correlated across at least one patient prescription drug data set.

In embodiments, the patient data derives from an electronic medical record. In embodiments, the patient data derives from a pharmacy database. In embodiments, the patient data derives from a laboratory database. In embodiments, the patient data derives from an insurer database. In embodiments, the patient data derives from a physician's database.

In embodiments, the physician data derives from a hospital. In embodiments, the physician data derives from a government data source. In embodiments, the prescription drug data set derives from a prescription drug monitoring program. In embodiments, the physician data includes insurance data. In embodiments, the insurance data includes data relating to prior insurance reimbursements for prescription medication.

In embodiments, a system for relationship discovery of physician prescription behavior, includes ingesting patient data received from a plurality of healthcare data providers and physician data from a plurality of physician practices; determining one or more relationships between the ingested patient data and physician data, wherein at least one new enriched data set is created based on the determined one or more relationships, wherein the enriched data set includes determined relationships with supplemental reference data; and generating an alert based at least in part on an indicator of aberrant prescription activity among the determined one or more relationships within the enriched data set.

In embodiments, the aberrant prescription activity generates a notice to a pharmacy to place a hold on fulfilling future prescriptions for a patient among the patient data. In embodiments, the supplemental reference data is financial data. In embodiments, the supplemental reference data is drug schedule data. In embodiments, the supplemental reference data is industry reference data. In embodiments, the supplemental reference data is prescription drug data. In embodiments, the supplemental reference data is geographic data. In embodiments, the supplemental reference data is toxicology data. In embodiments, the aberrant prescription activity is an unauthorized prescription fulfillment.

In embodiments, the method includes detecting misuse of a controlled medication of a patient, that includes obtaining, by a processing system, lab test results of a patient from a lab testing system; obtaining, by the processing system, patient attributes of the patient from one or more patient data sources; generating, by the processing system, a usage profile corresponding to the patient based on the lab test results of the patient and the patient attributes; determining, by the processing system, whether the usage profile is indicative of potential misuse of the controlled medication based on one or more features of the usage profile; and in response to determining potential misuse of the controlled medication, transmitting a notification that indicates the potential misuse by the patient.

In embodiments, the potential misuse of the controlled medication is overuse of the controlled medication. In embodiments, the potential misuse of the controlled medication is underuse of the controlled medication.

In embodiments, generating the usage profile includes combining multiple test results of the patient to obtain a history of lab results of the patient. In embodiments, the patient attributes include two or more of an age of the patient, a weight of the patient, a body type of the patient, and an activity level of a patient. In embodiments, the patient attributes are obtained from an electronic medical record database of a healthcare system associated with a clinic of the patient. In embodiments, the patient attributes are obtained from an insurer database of an insurance system associated with an insurance provider of the patient.

In embodiments, determining whether the usage profile is indicative of potential misuse includes identifying a set of features based on the usage profile; inputting the set of features into a machine learned classification model that is trained to classify instances of potential misuse of the classified medication; obtaining a classification from the machine learned classification model and a confidence score indicating a degree of confidence in the classification determined by the machine learned classification model; and determining whether the usage profile is indicative of the potential misuse based on the classification and the confidence score.

In embodiments, determining whether the usage profile is indicative of potential misuse includes identifying a set of features based on the usage profile; clustering the usage profile with a plurality of other usage profiles using a clustering algorithm, each other usage profile respectively corresponding to a respective previous patient that was prescribed the controlled medication and deemed either to be indicative of potential misuse of the controlled medication or of proper use of the controlled medication; determining a cluster of the usage profile of the patient to which the usage profile was clustered, wherein the cluster includes a subset of the plurality of other usage profiles; and determining whether the usage profile is indicative of potential misuse of the controlled medication based on the other usage profiles in the subset of the plurality of other usage profiles.

In embodiments, determining whether the usage profile is indicative of potential misuse includes identifying a set of features based on the usage profile; and applying a set of rules to the features to determine whether the usage profile is indicative of potential misuse.

In embodiments, the lab test results include results from a urine analysis test. In embodiments, the lab test results include results from a blood test. A method for recommending a lab test for a patient, that includes obtaining, by a processing system, a proposed prescription for the patient from an external data source, the proposed prescription indicating a medication; obtaining, by the processing system, patient attributes for the patient, including a diagnosis of the patient; determining, by the processing system, whether to recommend one or more different lab tests for the patient prior to the patient beginning the proposed prescription based on the proposed prescription and the patient attributes; and in response to determining to recommend one or more different lab tests for the patient, providing, by the processing system, a testing recommendation to a customer relationship management system, wherein the testing recommendation indicates the one or more tests that are recommended for the patient, wherein the customer relationship management system transmits the testing recommendation to a healthcare system of a clinic associated with the patient.

In embodiments, determining whether to recommend the one or more different lab tests includes identifying a set of features based on the proposed prescription and the patient attributes; inputting the set of features into one or more machine learned models that are respectively trained to determine whether to recommend a respective lab test; obtaining one or more respective recommendations from the one or more respective machine learned models based on the set of features, wherein each respective recommendation indicates whether the respective lab test should be performed for the patient given the patient attributes and has a confidence score that indicates a degree of confidence in the recommendation; and for each recommendation, determining whether to recommend the respective lab test indicated therein based on the confidence score of the recommendation.

In embodiments, the method includes each of the one or more machine learned models corresponds to the medication. In embodiments, the method includes each of the one or more machine learned models is trained on a plurality of training data samples that respectively correspond to a plurality of previous patients that were prescribed the medication, wherein each training data sample includes respective patient attributes of the respective previous patient and an outcome related to the medication for the previous patient. In embodiments, each training data sample further includes one or more lab test results of the respective previous patient.

In embodiments, determining whether to recommend the one or more different lab tests includes identifying a set of features based on the proposed prescription and the patient attributes; inputting the set of features into a machine learned model that is trained to determine whether to recommend one or more of a plurality of different lab tests given a set of patient attributes; obtaining a recommendation and a confidence score corresponding to the recommendation from the machine learned model based on the set of features, wherein the recommendation indicates any of the plurality of different lab tests that should be performed for the patient given the patient attributes, and the confidence score indicates a degree of confidence in the recommendation; and determining whether to accept the recommendation based on the confidence score of the recommendation.

In embodiments, the machine learned model corresponds to the medication. In embodiments, the machine learned model is trained on a plurality of training data samples that respectively correspond to a plurality of previous patients that were prescribed the medication, wherein each training data sample includes respective patient attributes of the respective previous patient and an outcome related to the medication for the previous patient.

In embodiments, a system for characterizing healthcare relationships, includes an interaction module identifying each sales and service representative and organization with whom a physician has interacted, the interaction module creating a physician interaction dataset; a machine learning module that identifies, within the physician interaction dataset, a plurality of relationships between each physician in the physician interaction dataset and each sales and service representative and organization; and a detection module for detecting that a previously identified relationship between at least one of a sales and service representative or organization has been broken; a correlation module that ensures that data within the physician interaction dataset are associated with the correct physician records; and a recommendation module to identify an alternative data path to form a new relationship with the patient data that was associated with the previously identified relationship.

In embodiments, the machine learning module is configured to train a machine learned model that is leveraged by a test management system. In embodiments, the machine learning module is configured to train a machine learned model that is leveraged by a prescription monitoring system. In embodiments, the machine learning module is configured to train a machine learned neural network model. In embodiments, the machine learned neural network model is a recurrent neural network model. In embodiments, the machine learning module is configured to train a Bayesian model. In embodiments, the machine learning module is configured to train an artificial intelligence system. In embodiments, the machine learning module is configured to train a rules-based recommendation system.

In embodiments, the rules-based recommendation system includes rules for determining the appropriateness of a treatment. In embodiments, the treatment is a prescription medication. In embodiments, the configuration of the machine learning module to train a rules-based recommendation system includes using training data from a prescription medication data set. In embodiments, the configuration of the machine learning module to train a rules-based recommendation system includes using training data from a prescription medication data set.

In embodiments, a method for monitoring prescription relationships, includes ingesting patient data received from one of a plurality of patient data providers, healthcare services data relating to the patient data, and data relating to physician prescription records; determining one or more relationships between the ingested patient data, healthcare services data, and physician prescription records, and previously ingested patient data, healthcare services data, and physician prescription records, wherein at least one new enriched data set is created based on the determined one or more relationships; extracting at least a portion of the patient data from the enriched data set in response to having received a request to generate a report associated with the patient data, wherein the extracted portion of the patient data is de-identified; aggregating the de-identified patient data as a function of the requested report; and presenting a visual representation of the de-identified patient data as a function of the aggregated, de-identified patient data and the requested report.

In embodiments, the de-identified patient data retains an identifier indicating an attending physician. In embodiments, the de-identified patient data retains an identifier indicating a patient insurer. In embodiments, the de-identified patient data retains an identifier indicating a pharmacy system.

In embodiments, the patient data derives from an electronic medical record. In embodiments, the patient data derives from a pharmacy database. In embodiments, the patient data derives from a laboratory database. In embodiments, the patient data derives from an insurer database.

In embodiments, a method for monitoring consistency in a drug prescription system, includes ingesting prescription drug data and prescription drug treatment plan data relating to a plurality of patients received from at least one of a plurality of patient data providers; determining, by the computing device, one or more relationships between the ingested prescription drug data and prescription drug treatment plan data relating to a plurality of patients and previously ingested prescription drug data and prescription drug treatment plan data, wherein at least one new enriched data set is created based on the determined one or more relationships; transmitting the enriched data set to a machine learning module; and using the machine learning module to compare treatment plans among the prescription drug treatment plan data, using simulations of one of one or more drugs and one or more treatment plans, among the prescription drug data and prescription drug treatment plan data.

In embodiments, the prescription drug data derives from an electronic medical record. In embodiments, the prescription drug data derives from a pharmacy database. In embodiments, the prescription drug data derives from a laboratory database.

In embodiments, the drug treatment plan derives from an insurer database. In embodiments, the drug treatment plan data derives from a physician's database.

In embodiments, the machine learning module is configured to train a machine learned model that is leveraged by a test management system. In embodiments, the machine learning module is configured to train a machine learned model that is leveraged by a prescription monitoring system. In embodiments, the machine learning module is configured to train a machine learned neural network model. In embodiments, the machine learned neural network model is a recurrent neural network model. In embodiments, the machine learning module is configured to train a Bayesian model. In embodiments, the machine learning module is configured to train an artificial intelligence system. In embodiments, the machine learning module is configured to train a rules-based recommendation system.

In embodiments, the rules-based recommendation system includes rules for determining the appropriateness of a treatment. In embodiments, the treatment is a prescription medication. In embodiments, the configuration of the machine learning module to train a rules-based recommendation system includes using training data from a prescription medication data set. In embodiments, the configuration of the machine learning module to train a rules-based recommendation system includes using training data from a prescription medication data set.

In embodiments, a system for monitoring consistency in a drug prescription system, includes a processor configured at least to identify a requested laboratory report; associate the laboratory report with a prescription drug management program; identify one or more laboratory result data; trigger a drug consistency awareness service corresponding to the prescription drug management program; send the one or more laboratory result data to a destination corresponding to the laboratory report; and send one or more parameters associated with the drug consistency awareness service to the destination corresponding to the laboratory report; a reporting module for intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction; and a recommendation module that provides the physician with an indicated likelihood that the patient is abusing and a risk report for the physician to work with the patient. In embodiments, the risk report includes a recommended treatment plan. In embodiments, the recommended treatment plan includes a recommended prescription medication.

In embodiments, a method for analyzing the quality or effectiveness of a laboratory, includes aggregating transaction data from a plurality of laboratories; analyzing volume and type of test from the transaction data; compiling a set of signals relating to pre-analytical, analytical, and post-analytical issues determined from the transaction data; parsing human-input information relating each of the issues determined from the transaction data; combining differently worded descriptions that are determined to have the same meaning; and automatically generating plain-language textual summaries that include at least a portion of detail from the issues determined from the transaction data.

In embodiments, the plain-language textual summaries include one or more details of the issues with a particular laboratory from the plurality of laboratories. In embodiments, the plain-language textual summaries include an improvement plan and gaps in care report for a particular laboratory from the plurality of laboratories.

In embodiments, mapping the issues determined from transaction information to an ontology entity module containing descriptions of medical entities and automatically generating an indication of a most likely medical entity whose actions was a cause of the one or more specified issues.

In embodiments, the one or more specified issues is over-utilization of a treatment. In embodiments, the one or more specified issues is under-utilization of a treatment. In embodiments, the one or more specified issues is prescription misuse. In embodiments, the one or more specified issues is a billing anomaly. In embodiments, the one or more specified issues is a denial of insurance reimbursement. In embodiments, the parsing human-input information relating each of the issues determined from the transaction data includes natural language processing.

In embodiments, a system for characterizing the activities of one or more physicians in a health care drug prescription system, includes an interception module for retrieving prescription drug data relating to a plurality of physicians; an interaction module identifying data associated with each sales and service representative with whom the one or more physicians have interacted; an ordering module identifying orders from each of a plurality of organizations by the plurality of physicians; and an analytic engine that associates the prescription drug data, data associated with each sales and service representative, and the orders with correct records for the plurality of physicians.

In embodiments, the method includes an insurance module that collects information from insurance records related to the one or more physicians. In embodiments, the method includes a hospital module that collects information from hospital records related to the one or more physicians.

In embodiments, the method includes an analytics module that determines whether lab ordering patterns of the physicians and indicates whether a subset of the ordering patterns is anomalous. In embodiments, the analytics module determined whether lab ordering patterns of the physicians are indicative of over utilization and/or appropriate utilization of lab resources based on best practices and/or clinical guidelines. In embodiments, the analytic engine includes a machine learning module.

In embodiments, the machine learning module infers relationships among the prescription drug data, data associated with each sales and service representative, and the orders. In embodiments, the machine learning module predicts a future relationship among the prescription drug data, data associated with each sales and service representative, and the orders. In embodiments, the prescription drug data derives from pharmacy data. In embodiments, the prescription drug data derives from insurer data.

In embodiments, a computerized method for healthcare data management, includes receiving health data from a plurality of healthcare communication sources, wherein the health data includes data related to an individual patient and data related to a population of patients; using a machine learning module to determine patterns related to effects of one or more of lifestyle, diagnosis, prognosis, present healthcare treatment, and previous healthcare treatment based on the health data of said individual patient and said population of patients; forming a digital twin of said individual patient based on the health data related to said individual patient, wherein the digital twin of said individual patient is a digital representation of at least one health state of said individual patient; forming a digital twin of said population of patients based on the health data related to said population of patients, wherein the digital twin of said population of patients is a digital representation of at least one health attribute of said population of patients; and presenting the digital twin of said individual patient, the digital twin of said population of patients, and data based on associations among the health data to one of said patient and said population of patients.

In embodiments, categorizing one or more patients according to one or more of lifestyle, diagnosis and/or prognosis, and present or previous healthcare treatments using a machine learning module, wherein the machine learning module applies fuzzy rules to categorize said one or more patients.

In embodiments, the healthcare data includes one or more social determinants of health and further comprising categorizing the one or more social determinants of health using the machine learning module.

In embodiments, outputting the digital twin of said patient and the digital twin of said population of patients to the machine learning module; simulating a future health state of said patient based on the digital twin of said patient using the digital twin of said patient and the machine learning module; simulating a future health state of said population of patients based on the digital twin of said population of patients using the digital twin of said population of patients and the machine learning module; updating the digital twin of said patient based on the simulation of the future health state of said patient; updating the digital twin of said population of patients based on the simulation of the future health state of said population of patients; and presenting to said user of the healthcare data system the updated digital twin of said patient and the updated digital twin of said population of patients, wherein said population of patients is determined based on one or more of lifestyle, diagnosis and/or prognosis, and present or previous healthcare treatments as categorized using the machine learning module.

In embodiments, simulation of the future health state of said first population of patients and/or the future health state of said second population of patients is performed according to simulation instructions received from one or more of said healthcare workers. In embodiments, simulation of the future health state of said first population of patients and/or the future health state of said second population of patients is performed according to simulation instructions formed by the machine learning module. In embodiments, said patient is a member of said population of patients and further comprising comparing the digital twin of said patient to the digital twin of said population of patients using the machine learning module. In embodiments, the machine learning module applies at least one of a batch gradient descent and a stochastic gradient descent to categorize said one or more patients.

In embodiments, the method includes further comprising comparing the digital twin of said individual patient with said health information to identify one or more gaps in care provided to said individual patient. In embodiments, the one or more gaps in care include failure by one or more healthcare professionals to follow one or more established clinical standards of care in treating said patient.

In embodiments, the method includes further comprising comparing the digital twin of said individual patient with said health information to identify one or more gaps in care provided to said population of patients. In embodiments, the one or more gaps in care include failure by one or more healthcare professionals to follow one or more established clinical standards of care in treating said population of patients.

In embodiments, a computerized method for healthcare data management, includes receiving health data from one or more healthcare communication sources, wherein the health data includes data related to a plurality of physicians and their interactions with a plurality of sales representatives; receiving sales data related to money spent on a plurality of pharmaceuticals by the plurality of physicians; forming a digital twin of at least one individual physician among the plurality of physicians based on the health data, wherein the digital twin of said individual physician is a digital representation of the physician's interactions with the plurality of sales representatives; forming a digital twin of at least one individual sales representative among the plurality of sales representatives based on the health data, wherein the digital twin of said individual sales representative is a digital representation of the sales representative's interactions with the plurality of physicians; presenting the digital twin of said individual physician and the digital twin of said individual sales representative; and determining a return on investment metric indicative of an amount of money spent versus an amount of money recovered by one or more of said individual physician, said individual sales representative, based on said health data, said sales data, and one or both of the digital twins of said individual physician and said individual sales representative.

In embodiments, the method includes receiving investment data related to costs of care by one or more of the said healthcare provider, said healthcare researcher, and said health insurance provider. In embodiments, the method includes determining the return on investment metric, wherein the return on investment metric is at least partially based on costs of care provided by one or more of said healthcare researcher and said health insurance provider based on said health information, said investment data, and one or both of the digital twins of said patient and said population of patients.

In embodiments, the method includes determining, using a machine learning module, whether providing a first treatment to said patient and/or said population of patients rather than providing a second treatment to said patient and/or said population of patients may result in an improved return on investment metric. In embodiments, the method includes determining an effect of a pre-existing condition on the return on investment metric of one of said patient and said population of patients.

In embodiments, the method includes outputting the digital twin of said patient and the digital twin of said population of patients to a machine learning module of the healthcare data system; simulating a future health state of said first population of patients based on the digital twin of said patient via the digital twin of said patient and the machine learning module; simulating a future health state of said second population of patients based on the digital twin of said population of patients via the digital twin of said population of patients and the machine learning module; updating the digital twin of said patient based on the simulation of the future health state of said patient; updating the digital twin of said population of patients based on the simulation of the future health state of said population of patients; and presenting to each of the healthcare workers, at the healthcare data system computing device, the healthcare research information determined to be relevant to at least one of said individual patient, said first population of patients, and said second population of patients.

In embodiments, simulation of the future health state of said first population of patients and/or the future health state of said second population of patients is performed according to simulation instructions received from one or more of said healthcare workers. In embodiments, simulation of the future health state of said first population of patients and/or the future health state of said second population of patients is performed according to simulation instructions formed by the machine learning module.

In embodiments, a system is provided for characterizing the activities of one or more patients in a health care system, including an interception module for retrieving prescription drug data relating to the one or more patients; a correlation module that ensures that the prescription drug data is associated with the correct records of the one or more patients, and an analytics module that determines whether prescription ordering patterns for the one or more patients and indicates whether a subset of the ordering patterns is anomalous as compared with a stored ordering criterion.

In embodiments, the method includes further comprising a waste module that determines whether the one or more patients have taken one of unnecessary and redundant tests.

In embodiments, the method includes a prediction module that analyzes tests taken by the one or more patients results of the tests, and comparisons with aggregate information, and recommends additional tests for the one or more patients in order to detect additional conditions.

In embodiments, the method includes a machine learning module that infers relationships among prescription orders. In embodiments, the method includes further comprising an artificial intelligence module that simulates future relationships among prescription orders.

In embodiments, a computerized method for healthcare data management, includes receiving health data from one or more healthcare communication sources, wherein the health information includes data related to an individual patient and data related to a population of patients; forming a digital twin of said individual patient based on the health data related to said individual patient, wherein the digital twin of said individual patient is a digital representation of at least one health state of said individual patient; forming a digital twin of said population of patients based on the health data related to said population of patients, wherein the digital twin of said population of patients is a digital representation of at least one health attribute of said population of patients; determining whether said population of patients have one or more symptoms similar to said patient; simulating, using a machine learning module, a future health state of the individual patient; detecting a new health state of the individual patient based at least in part on new health data received; and transmitting an alert to the at least one healthcare provider indicating a discrepancy between the simulated future health state and the new health state.

In embodiments, the machine learning simulation includes pharmaceutical data to simulate a future health state contingent upon the patient following a specified treatment plan. In embodiments, the machine learning simulation includes treatment plan data to simulate a future health state contingent upon the patient receiving a stated medication. In embodiments, the machine learning simulation uses a digital twin of the patient. In embodiments, the machine learning simulation uses a plurality of digital twins of the patient.

In embodiments, simulation of the new health state is based in part on a measured health state of a population of patients matched to the individual patient according to a criterion. In embodiments, simulation of the new health state is based in part on a simulated health state of a population of patients matched to the individual patient according to a criterion.

In embodiments, the method includes simulating, using the machine learning module, effects of at least one of one or more drug treatment options of said individual patient, wherein the drug treatment options vary by the timing of providing mediation to the individual patient.

In embodiments, the method includes simulating, using the machine learning module, effects of at least one of one or more drug treatment options of said individual patient, wherein the drug treatment options vary by dosage level of mediation to the individual patient.

In embodiments, the method includes receiving healthcare study information including at least one of methodology and results of one or more healthcare studies; and comparing, using the machine learning module, the healthcare study information to simulations of one or more said drug treatment options to determine at least one of reliability and consistency of the simulations of one or more said drug treatment options.

In embodiments, the method includes simulating application of best clinical practices for a desired clinical outcome on said individual patient via the digital twin of said individual patient.

In embodiments, the method includes receiving simulation instructions, the simulation instructions including one or more research experiments; simulating the one or more research experiments, and results of best clinical practices on at least one of said individual patient and a population of patients using at least one of the digital twin of said individual patient and the digital twin of said population of patients.

In embodiments, the method includes receiving simulation instructions, the simulation instructions including one or more drug treatment regimens; simulating the one or more drug treatment regimens on one or both of said individual patient and a population of patients using at least one of the digital twin of said individual patient and the digital twin of said population of patients. In embodiments, simulation of said individual patient and/or said population of patients is performed according to simulation instructions received from one or more of healthcare workers. In embodiments, simulation of said individual patient and/or said population of patients is performed according to simulation instructions formed by the machine learning module.

A more complete understanding of the disclosure will be appreciated from the description and accompanying drawings and the claims, which follow.

### BRIEF DESCRIPTION OF THE DRAWINGS

The accompanying drawings, which are included to provide a better understanding of the disclosure, illustrate embodiments of the disclosure and, together with the description, serve to explain the principle of the disclosure. In the drawings:

FIG. 1 is a schematic illustrating an example environment of a pharmacological tracking platform according to some embodiments of the present disclosure;

FIG. 2 is a schematic illustrating an example set of components of a customer relationship management (CRM) system of a pharmacological tracking platform according to some embodiments of the present disclosure;

FIG. 3 is a schematic illustrating an example set of components of a test management system of a pharmacological tracking platform according to some embodiments of the present disclosure;

FIG. 4 is a schematic illustrating an example set of components of a prescription monitoring system of a pharmacological tracking platform according to some embodiments of the present disclosure;

FIG. 5 is a schematic illustrating an example reporting system environment of a pharmacological tracking platform according to some embodiments of the present disclosure;

FIG. 6 is an illustration of an example of an enhanced toxicology report according to some embodiments of the present disclosure;

FIG. 7 is an illustration of another example of an enhanced toxicology report according to some embodiments of the present disclosure;

FIG. 8 is an illustration of yet another example of an enhanced toxicology report according to some embodiments of the present disclosure;

FIG. 9 is an illustration of a further example of an enhanced toxicology report according to some embodiments of the present disclosure;

FIG. 10 is an illustration of an additional example of an enhanced toxicology report according to some embodiments of the present disclosure;

FIG. 11 is a diagram of an example computing system including an example computing device and an example server computing device according to some implementations of the present disclosure;

FIG. 12 is a functional block diagram of the example computing device of FIG. 11;

FIG. 13 is a schematic illustrating an example environment of a pharmacological tracking platform having one or more digital twin modules according to some embodiments of the present disclosure;

FIG. 14 illustrates a simplified view of the health monitoring command center module and the platform in relation to an employer and its employee, a medical lab and a general human resource information system according to some implementations of the present disclosure;

FIG. 15 illustrates a subset of functions performed by the health monitoring command center according to some implementations of the present disclosure;

FIG. 16 illustrates a simplified example workflow of an employee's interaction with the health monitoring command center according to some implementations of the present disclosure;

FIG. 17 illustrates a simplified view of how a person may interact with the health monitoring command center via a computing device and complete a symptom questionnaire at an entrance checkpoint to a workplace according to some implementations of the present disclosure;

FIG. 18 illustrates how a symptoms summary, lab results, and LRI that is associated with an individual may be tracked by the individual using a mobile application in communication with the health monitoring command center according to some implementations of the present disclosure;

FIG. 19 illustrates a simplified example of contact tracing using the health monitoring command center according to some implementations of the present disclosure;

FIG. 20 illustrates the health monitoring command center receiving test results and other data from a plurality of testing sites and types according to some implementations of the present disclosure;

FIG. 21 illustrates a hypothetical dashboard of the health monitoring command center displaying the results for a particular work site according to some implementations of the present disclosure;

FIG. 22 illustrates an example dashboard view of an employee roster and the corresponding health status indicators according to some implementations of the present disclosure;

FIG. 23 illustrates the health monitoring command center presenting a dashboard view of an individual's summary data according to some implementations of the present disclosure; and

FIG. 24 illustrates a simplified view of a person's detailed symptom and testing history, indicating the dates on which symptoms and/or testing events occurred and the results of each occurrence according to some implementations of the present disclosure.

### DETAILED DESCRIPTION

As mentioned above, there is a need for improved methods and systems for detecting and addressing situations involving improper prescription of medication, improper utilization of prescribed medications, and diversion of prescribed medications to unprescribed uses. As mentioned above, there is a need for improved methods and systems for detecting and addressing situations involving improper prescription of medication, improper utilization of prescribed medications, and diversion of prescribed medications to unprescribed uses. In the United States, for example, prescription drug monitoring programs (PDMPs) are utilized to track prescriptions of controlled drugs.

In order to address the above noted need for improved methods and systems for detecting and addressing situations involving improper prescription of medication, improper utilization of prescribed medications, and diversion of prescribed medications to unprescribed uses, the present disclosure is directed to an improved pharmacological tracking platform. The pharmacological tracking platform can be utilized to perform various functions, including but not limited to a report generation and outputting function, a misuse of a controlled medication function, and a laboratory test recommendation function. Each of these functions can be performed separately or in various combinations to address the above noted and other needs associated with the goal of preventing adverse drug-related events.

With respect to the report generation and outputting function, the present disclosure provides for generating an enhanced toxicology report corresponding to a patient. The enhanced toxicology report is designed as a simple and easy to understand summary of the use and potential misuse of controlled substances for a patient. As more fully described herein, the enhanced toxicology report can include various graphical elements to present information related to the use and potential misuse of controlled substances for a patient. These graphical elements can include, but are not limited to, graphs of historical trends or changes over time, a graph of prescriptions issued to the patient by prescriber by time periods, numerical scores, and color indicators. The enhanced toxicology report can be requested by prescribers, pharmacists, and other healthcare professionals to assist in treating a patient, as more fully described below.

In order to generate the enhanced toxicology report, laboratory test results from a laboratory and controlled substance prescription data for the patient are analyzed. The laboratory test results are indicative of a toxicology screen of the patient and the controlled substance prescription data includes prescriptions of controlled substances issued to the patient for a relevant time period. From this information, a daily morphine milligram equivalent of the patient for a given time period, an overdose risk score, and a drug consistency assessment are determined. The daily morphine milligram equivalent of the patient for the given time period corresponds to a cumulative intake of opioid class drugs by the patient on a daily basis for the given time period. The overdose risk score is indicative of a likelihood of an unintentional overdose by the patient, and the drug consistency assessment is representative of a match between the controlled substance prescription data and the laboratory test results for the patient. It should be appreciated that other scores, assessments, measurements, calculations, etc. can be determined.

With respect to the misuse of controlled medication function, the present disclosure provides for techniques for determining whether a usage profile of a patient is indicative of potential misuse of one or more controlled medications. A machine learning model or other forms of artificial intelligence are utilized to generate a potential misuse score or similar measurement of the likelihood that a patient is or has the potential for misusing a controlled substance. In some aspects, laboratory test results from a laboratory that are indicative of a toxicology screen of the patient are utilized, in conjunction with patient attributes of the patient, to generate the usage profile of the patient. Various features of the usage profile can be utilized with the artificial intelligence system to determine the likelihood that the patient is or has the potential for misusing a controlled substance. In response to determining that the patient is or has the potential for misusing a controlled substance, or when a healthcare professional is otherwise treating the patient, a notification or report of the patient's potential for misusing a controlled substance can be provided in order to assist with the treatment of the patient.

With respect to the laboratory test recommendation function, the present disclosure provides for techniques for determining whether to recommend one or more laboratory tests for a patient, e.g., at a time of prescribing a controlled substance. A machine learning model or other forms of artificial intelligence are utilized to generate a laboratory test recommendation or similar measurement of the likelihood that the patient would benefit from one or more specific laboratory tests before the patient is given a proposed prescription. In some aspects, a proposed prescription for the patient is utilized, in conjunction with patient attributes of the patient (e.g., a diagnosis), to determine whether to recommend one or more specific laboratory tests. Various features of the proposed prescription and patient attributes can be utilized with the artificial intelligence system to determine the likelihood that the patient would benefit from a specific laboratory test before beginning the prescription of the controlled substance. In response to determining that the patient would benefit from one or more specific laboratory tests, or when a healthcare professional is otherwise treating the patient, a notification, report, or laboratory test recommendation for the patient can be provided in order to assist with the treatment of the patient.

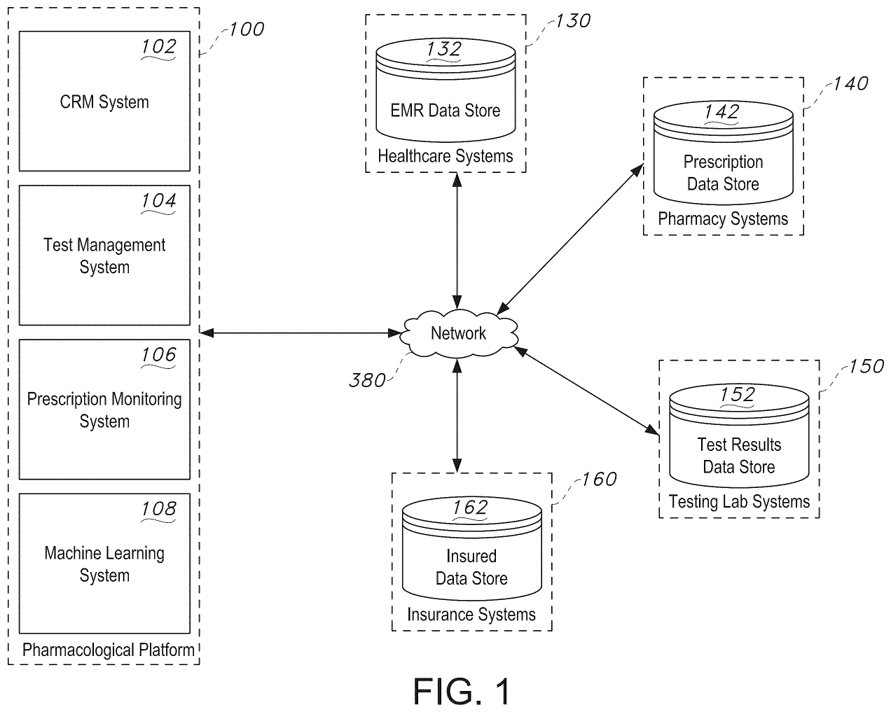

FIG. 1 illustrates an example pharmacological tracking platform 100 according to some embodiments of the present disclosure. In embodiments, the pharmacological tracking platform 100 is configured to collect and monitor data relating to laboratory tests (“lab tests” or “tests”) collected in connection with a treatment of a patient and/or data relating to prescription medications that are prescribed to patients. The pharmacological tracking platform 100 may obtain data from multiple external sources, including electronic medical records (EMRs), insurer databases, pharmacy databases, testing lab databases, prescription drug monitoring programs, and/or other suitable data sources. The pharmacological tracking platform 100 may use the obtained data to: make recommendations relating to the types of lab tests patients should undertake before beginning a potential prescription; determine whether a patient may be misusing a controlled medication (e.g., an opiate, a benzodiazepine, or amphetamine); determine whether a physician is overprescribing a controlled medication; determine whether a physician or clinic is over-ordering or under-ordering lab tests for their patients; and/or assess the quality of a testing lab. The pharmacological tracking platform 100 may perform additional or alternative tasks without departing from the scope of the disclosure.

As shown in FIG. 1, the pharmacological tracking platform 100 may communicate with external sources such as electronic medical records (EMRs), insurer databases, pharmacy databases, testing lab databases, and/or prescription drug monitoring programs, as well as other computing device(s), systems, data sources, applications, and platforms, via a network 380. It should be appreciated that the network 380 can take the form of any communication network suitable for communicatively linking computing devices and/or components thereof, including, without limitation, a virtual private network, the Internet, a Local Area Network, a Wide Area Network, a cellular network, and an intranet or other private networks.

In embodiments, the pharmacological tracking platform 100 may use the collected data to determine whether a patient should have one or more lab tests ordered prior to beginning a proposed prescription. In some of these embodiments, the platform 100 may obtain data relating to the patient, including the proposed prescription, as well as outcome data from previous patients that have taken the prescription in the past that includes lab tests associated with those patients, attributes of those patients (e.g., age, sex, weight, body type), and the result of the treatment (e.g., was the prescription effective). In this way, the patient may be prescribed a medication that is more likely to be effective prior to beginning the treatment. Furthermore, in embodiments, the pharmacological tracking platform 100 may recommend one or more different tests for the patient during or after the treatment, to ensure that the patient is receiving effective treatment.

In embodiments, the pharmacological tracking platform 100 may monitor test results of respective patients to determine whether the respective patients are misusing a controlled medication (e.g., an opiate, a benzodiazepine, or an amphetamine). Misusing a controlled medication may include overusing/abusing the medication, underusing the medication (which may be indicative of someone illegally distributing the medication), using the medication with other controlled medications, or improperly using the medication (e.g., not taking the medication at the correct times). In some of these embodiments, the pharmacological tracking platform 100 may analyze a patient's lab test results (e.g., toxicology screens such as blood tests or urine analysis tests) to determine if the patient is potentially misusing a controlled medication. In embodiments, the pharmacological tracking platform 100 may consider a patient's lab tests in their totality (e.g., over the period when the patient is prescribed the medication) and/or in view of lab tests of other patients (e.g., lab tests of patients that properly use the medication and lab tests of patients that misuse the medication) and attributes of those patients.

In embodiments, the pharmacological tracking platform 100 may provide notifications and/or recommendations to appropriate third parties, such as healthcare organizations (e.g., hospitals and/or clinics), physicians, pharmacies, insurers, and the like. In some of these embodiments, the platform 100 may provide customer relationship management capabilities, whereby the platform 100 may leverage these capabilities to provide the notifications and/or recommendations.

In embodiments, the pharmacological tracking platform 100 may include a customer relationship management (CRM) system 102, a test management system 104, a prescription monitoring system 106, and/or a machine learning system 108. The pharmacological tracking platform 100 may include additional or alternative systems without departing from the scope of the disclosure.

In embodiments, the test management system 104 may determine whether to recommend lab testing for a patient given a proposed treatment of the patient. In response to the test management system 104 determining to recommend lab testing for a patient, the CRM system 102 may provide a mechanism (e.g., a GUI) by which a user (e.g., a representative of a lab testing organization) may provide the notification recommending lab testing to a healthcare provider (e.g., the treating physician or the office thereof), a pharmacy, and/or an insurance provider. In embodiments, the test management system 104 may also perform various analytics on captured data to determine when physicians are overusing or underusing lab tests in their respective practices. In these embodiments, the test management system 104 may monitor the ordering of tests from a group of physicians to determine instances where a physician's ordering of tests is anomalous. The test management system 104 may provide other features as well, such as quality assessment relating to testing labs.

In embodiments, the prescription monitoring system 106 monitors test results of patients prescribed certain prescription medications to determine whether the respective patients are misusing the prescription medication (e.g., overusing/abusing, or underusing the prescription medication). In response to the prescription monitoring system 106 determining a likely case of misuse, the CRM system 102 may provide a mechanism by which a user (e.g., a representative of a lab testing organization) may provide the notification of potential misuse to a healthcare provider (e.g., the treating physician or the office thereof), a pharmacy, and/or an insurance provider.

In embodiments, the CRM system 102 may be accessed by users associated with a testing lab system 150. In embodiments, the CRM system 102 may allow these users to manage relationships and communications with healthcare providers associated with healthcare systems 130, pharmacy employees associated with pharmacy systems 140, and/or insurance providers associated with insurance systems 160. In embodiments, the CRM system 102 may receive recommendations and/or notifications from the test management system 104 and/or the prescription monitoring system 106. The CRM system 102 may perform additional or alternative tasks, such as obtaining data from external data sources (e.g., healthcare systems 130, pharmacy systems 140, testing lab systems 150, and/or insurance system 160) and may structure the obtained data into different types of records according to respective schemas.

In embodiments, the pharmacological tracking platform 100 may include a machine learning system 108 that is configured to train machine learned models that are leveraged by the test management system 104 and/or the prescription monitoring system 106. The machine learning system 108 may train any suitable type of model, including neural networks, deep neural networks, recurrent neural networks, Hidden Markov Models, Bayesian models, regression models, and the like. The machine learning system 108 may train the models in a supervised, unsupervised, or semi-supervised manner. In embodiments, the machine learning system 108 may collect training data from one or more data sources. Depending on the purpose of the model, the data types included in the training data will vary. For example, models used to recommend testing for a patient prior to the patient undergoing a particular treatment (e.g., prescription) may be trained on training data that includes prescription data of respective patients, outcome data relating to the respective patients' treatments, and lab test results of the respective patients that correspond to the outcome data. Models used to classify a patient's misuse of a medication may be trained on training data that includes lab test results of patients that were deemed to be misusing a particular medication and patient information relating to those patients, and lab test results of patients that were deemed to be using the medication properly and patient information relating to those patients.

A healthcare system 130 may refer to a collection of one or more computing devices, including client user devices and/or server devices that are used in connection with a healthcare organization (e.g., one or more hospitals, doctor offices, etc.). In embodiments, a healthcare system 130 may include an EMR data store 132. An EMR data store 132 may include one or more databases that store and/or index electronic medical records. A respective electronic medical record may store or reference patient data of a respective patient of the healthcare organization. An electronic medical record may include a patient identifier, one or more physician identifiers that indicate respective physicians of a patient, physician notes relating to the patient, prescription data indicating treatments that were prescribed to a patient, test results of the patient, and the like. The EMR data store 132 may store additional or alternative data without departing from the scope of the disclosure.

A pharmacy system 140 may refer to a collection of one or more computing devices, including client user devices and/or server devices that are used in connection with a pharmacy organization (e.g., a pharmacy or chain of pharmacies). In embodiments, the pharmacy system 140 may include a prescription data store 142. The prescription data store 142 may include one or more databases that store and/or index prescription records. A respective prescription record may store a prescription ID that uniquely identifies a prescription of a patient, a patient ID identifying the patient to whom the prescription corresponds, a physician ID that identifies the physician that wrote the prescription, a medication ID that identifies the medication that was prescribed, a quantity of the medication that is prescribed, a dosage of the medication that is prescribed, a date on which the medication was prescribed, a date on which the prescription expires, and the like. The prescription data store 142 may store additional or alternative data without departing from the scope of the disclosure.

An insurance system 160 may refer to a collection of one or more computing devices, including client user devices and/or server devices that are used in connection with an insurance organization (e.g., a health insurance provider). The insurance system 160 may be configured to process claims, payout medical bills, collect medical data relating to insured patients, and the like. In embodiments, an insurance system 160 may include an insured data store 1622. The insured data store 1622 may include one or more databases that store and/or index insured records. A respective insured record corresponds to a respective insured person and may store an insured ID that uniquely identifies the person being insured, a policy ID that identifies the policy of the insured, one or more healthcare system IDs that identify one or more respective healthcare systems that the insured visits or has visited, one or more physician IDs that respectively identify a physician of the insured, one or more prescription IDs that respectively identify one or more respective prescriptions of the insured, billing information of the patient, a medical history of the patient, and the like. The insured data store 1622 may store additional or alternative data without departing from the scope of the disclosure.

A testing lab system 150 may refer to a collection of one or more computing devices, including client user devices and/or server devices that are used in connection with an organization that performs medical testing (e.g., a blood testing, a urine analysis, genetic testing, and the like). In embodiments, the testing lab system 150 may include a test results data store 152. The test results data store 152 may include one or more databases that store and/or index test result records that respectively correspond to testing administered by the organization. In embodiments, a test results record may include a test ID that identifies the test that was performed, a test type ID that identifies the type of test performed, a subject ID that indicates a subject of the test (e.g., a patient), a requestor ID that indicates an organization that ordered the test (e.g., a healthcare organization or physician), results data (e.g., the results of the test), and a date (e.g., a date on which the test was administered). The test results data store 152 may store additional or alternative data without departing from the scope of the disclosure.

In embodiments, the CRM system 102 is configured to provide a framework for users associated with a testing lab organization to manage relationships with healthcare organizations, insurance organizations, and/or pharmacy organizations. In embodiments, the CRM system 102 may allow a user to send communications (e.g., emails, text messages, directed messages on social media platforms, and the like) to a healthcare provider, pharmacy, and/or insurance provider. In embodiments, the CRM system 102 may notify a user of the CRM system 102 when a healthcare provider is prescribing a treatment that may benefit from a test (e.g., a genetic test, a blood test, a urine test, etc.). For example, the test management system 104 (discussed below) may recommend the patient undergoing a genetic test before beginning a specific medication to see if the specific medication is likely to be effective in treating the patient suffering from a specific ailment. In these scenarios, the user may opt to send the recommendation to a healthcare provider (e.g., treating physician), the pharmacy, and/or the insurance provider of the patient.

In embodiments, the CRM system 102 may allow a user to send communications (e.g., emails, text messages, directed messages, and the like) to a third party when a patient is suspected of misusing a prescription medication (e.g., overusing/abusing or underusing the prescription medication). In these embodiments, the CRM system 102 may receive a notification of a detected misuse. In response, the CRM system 102 may identify a healthcare provider of the patient misusing a medication and may transmit a notification to the healthcare provider indicating the detected misuse. In some embodiments, the notification to the healthcare provider is sent automatically upon a detected misuse. In some embodiments, a user associated with the testing lab may be provided the option of sending the notification to the healthcare provider.

In embodiments, the test management system 104 is configured to assist users to identify patients that should undergo tests in connection with a prescribed treatment. For example, the test management system 104 may recommend a patient undergo genetic testing in response to a patient being prescribed a particular medication given the particular medication, one or more attributes of the patient, and the ailment of the patient. In this way, the patient may be prescribed the correct medication, rather than have a period where the patient is prescribed a treatment that is unlikely to be effective.

In embodiments, the test management system 104 leverages one or more machine learned models to determine whether to recommend testing for a patient that has been prescribed a specific treatment. In some of these embodiments, the test management system 104 may receive a prescribed treatment (e.g., a medication identifier) for the patient and one or more attributes of the patient (e.g., a patient's age, a patient's sex, a patient's body type, a patient's other medications). The test management system 104 may input these features into a machine learned model that determines whether a particular test (e.g., a genetic test, a blood test, etc.) should be performed. In these embodiments, the test management system 104 may leverage multiple models, such that each respective model may correspond to a different type of test. In some embodiments, the test management system 104 may receive a prescribed treatment (e.g., a medication identifier) for the patient and one or more attributes of the patient (e.g., a patient's age, a patient's sex, a patient's body type, a patient's other medications) and may input these features into a machine learned model that determines any tests that should be performed.

In embodiments, the test management system 104 may be configured to employ a rules-based approach to determine whether any tests should be performed on a patient given a prescribed medication. In these embodiments, the test management system 104 may assess the patient with a set of rules that correspond to a medication that is prescribed to the patient. The conditions which trigger certain rules may be learned using analytics that are derived from outcomes relating to the medication. For example, a certain medication may be ineffective for people over the age of sixty having a certain genetic characteristic, but effective for all other segments of the population. In this example, if the patient is over the age of sixty, the test management system 104 may determine that the patient should undergo genetic testing to determine whether the patient exhibits the certain genetic characteristic.

Upon determining that one or more tests should be performed for a patient, the test management system 104 may output the recommended tests to the CRM system 102. As discussed, in embodiments, a user associated with the lab testing organization may elect to provide the recommendation to an appropriate recipient. In other embodiments, the CRM system 102 may provide the recommendation to the appropriate recipient automatically.

In embodiments, the prescription monitoring system 106 receives lab test results for patients (e.g., blood tests, urine analysis tests) and determines whether it is likely that the respective patients are misusing a prescription medication. In some embodiments, the prescription monitoring system 106 may obtain lab test results of a patient, medication identifiers of any prescription medications that the patient is prescribed or currently using, and relevant patient data (e.g., an ailment of the patient, the age of the patient, weight of the patient, height of the patient, body fat percentage of the patient, and the like). The prescription monitoring system 106 may then determine whether a patient is misusing a prescription medication based on the lab test results, the medication identifiers, and the relevant patient data.

In embodiments, the prescription monitoring system 106 may utilize machine learning and AI techniques to determine if a patient is misusing a prescription medication. In some of these embodiments, the prescription monitoring system 106 may leverage one or more machine learned models to determine if a patient is misusing a prescription medication. For example, in embodiments, a machine learned model may be trained to identify when a patient is likely abusing a particular prescription medication (e.g., an opiate, a benzodiazepine, or amphetaminc). These models may be trained on training data samples relating to patients that were determined to be abusing the medication and patients that were determined to be using the prescription medication properly. In these embodiments, the prescription monitoring system 106 may obtain lab test results of the patient (e.g., a blood test and/or a urine analysis test), a prescription medication that the patient is being prescribed, and relevant patient information (e.g., an ailment of the patient, other prescriptions of the patient, an age of the patient, a gender of the patient, a weight of the patient, a body fat percentage of the patient, and the like). In embodiments, the machine learned model may output a classification relating to the patient that indicates whether the patient is likely abusing the medication or using the medication properly. In embodiments, the classification may include a confidence score, whereby a higher confidence score indicates a higher degree of confidence in the classification. In some of these embodiments, the machine learned model(s) may be trained to identify the type of abuse of a medication (e.g., overuse/addiction, use with other controlled substances, and the like). In embodiments, the prescription monitoring system 106 may leverage other machine learned models. For instance, the prescription monitoring system 106 may leverage a machine learned model that is trained to identify when a patient is underusing a prescription medication, which may be indicative of a patient illegally distributing the prescription medication to other people.

In some embodiments, the prescription monitoring system 106 may be configured to apply a rules-based approach for detecting misuse. In these embodiments, the prescription monitoring system 106 may obtain lab test results of the patient (e.g., a blood test and/or a urine analysis test), a prescription medication that the patient is being prescribed, and relevant patient data (e.g., an ailment of the patient, other prescriptions of the patient, an age of the patient, a gender of the patient, a weight of the patient, a body fat percentage of the patient, and the like), which may be analyzed in view of a set of one or more conditions. In these embodiments, the one or more conditions may be indicative of one or more types of abuse. For example, conditions may define maximum allowances of detected opiates, benzodiazepines, and/or amphetamines in a patient's blood or urine. These allowances may vary depending on the patient's prescription and metabolic factors (e.g., ailments, age, body fat, weight, etc.). For example, conditions for a patient with a relatively higher prescribed dosage and/or a relatively higher body fat percentage may define relatively higher allowances. Similarly, conditions may define minimum levels of detected opiates, benzodiazepines, and/or amphetamines for patients, which may vary based on one or more factors (e.g., prescribed amounts and/or metabolic factors). In these examples, the conditions may be tailored to identify underuse of the prescription medication. Upon receiving one or more test results and/or a notification of a prescription relating to a patient, the prescription monitoring system 106 may apply the one or more rules to determine if the patient is misusing the prescription medication.

Upon determining that a patient is likely misusing a prescription medication, the prescription monitoring system 106 may output a notification of the detected misuse to the CRM system 102. As discussed, in embodiments, a user associated with the lab testing organization may elect to provide the notification to an appropriate recipient(s) (e.g., the treating physician) or the CRM system 102 may provide the notification to the appropriate recipient(s) automatically.

The prescription monitoring system 106 may monitor other entities as well. For example, in embodiments, the prescription monitoring system 106 may monitor a physician's prescription history to determine if the physician if overprescribing certain medications.

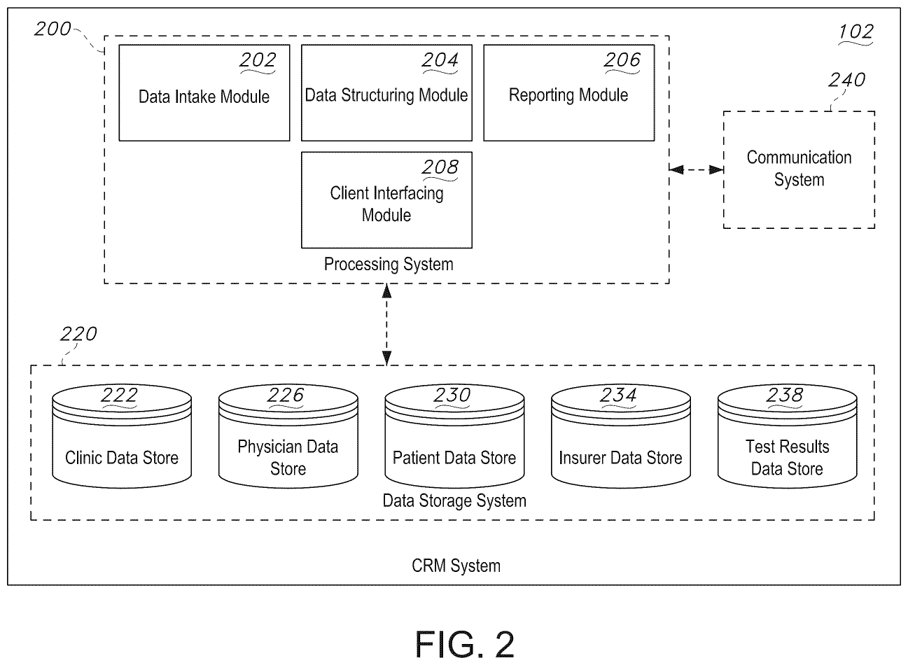

FIG. 2 illustrates a set of example components of a CRM system 102 according to some embodiments of the present disclosure. In embodiments, the CRM system 102 may include a processing system 200, a data storage system 220, and a communication system 240. The processing system 200 may include memory (e.g., RAM and/or ROM) that stores computer-executable instructions. In embodiments, the processing system 200 executes a data intake module 202, a data structuring module 204, a reporting module 206, and/or a client interfacing module 208, each of which is discussed in further detail below. The processing system 200 may execute additional or alternative modules without departing from the scope of the disclosure. The data storage system 220 may include one or more storage devices (e.g., hard disk drive, flash drive, etc.) that store data. In embodiments, the data storage system 220 may store a clinic data store 222, a physician data store 226, a patient data store 230, an insurer data store 234, and a test results data store 238, all of which are discussed in further detail below. The communication system 240 may include one or more communication devices that interface with a communication network (e.g., the internet). The one or more communication devices may effectuate wired or wireless communication.

In embodiments, the clinic data store 222 stores clinic related data. A clinic may refer to any sized healthcare organization (e.g., a hospital system, a multi-physician office, a single physician office) and/or units within an organization (e.g., a cardio-unit of a hospital, an oncology unit of a hospital, a surgery unit of a hospital, an intensive care unit of a hospital, etc.). In embodiments, the clinic data store 222 stores a clinic database that stores and/or indexes clinic records. Each clinic record may correspond to a respective clinic. A clinic record may include and/or may be related to a clinic ID that identifies the clinic, one or more physician IDs that identify physicians associated with the clinic, one or more administrator IDs that identify administrators associated with the clinic (e.g., contacts/decision makers at the clinic), ordering data that indicates orders made by the clinic (e.g., lab tests ordered by the clinic, the lab testing organization that performed each lab test, and dates of each order), and/or suitable metadata. It is noted that the list of items included in a clinic record is provided for example only and may include additional or alternative types of data.

In embodiments, the physician data store 226 stores physician-related data. In embodiments, the physician data store 226 stores a physician database that stores and/or indexes physician records. Each physician record may correspond to a respective physician or other healthcare providers. A physician record may include and/or may be related to a physician ID that identifies the physician, a set of patient IDs that identify a set of patients that see the physician, a set of clinic IDs that indicate any clinics that the physician is associated with, ordering data that indicates orders made by the physician (e.g., lab tests ordered by the physician, the lab testing organization that performed each lab test, and dates of each order), prescription data indicating prescriptions written by the physician (including, dosages, amounts, and dates of each prescription), and/or suitable metadata. It is noted that the list of items included in a physician record is provided for example and may include additional or alternative types of data.

In embodiments, the patient data store 230 stores patient-related data. In embodiments, the patient data store 230 stores a patient database that stores and/or indexes patient records. Each patient record may correspond to a respective patient. A patient record may include and/or may be related to a patient ID that identifies the patient, a set of physician IDs that identify a set of physicians that treat or have treated the patient, a set of clinic IDs that indicate any clinics that the patient has visited, ordered test data that indicates any tests ordered for the patient (e.g., lab tests ordered for the patient, the results of the tests, the lab testing organization that performed each lab test, and the date of each test), prescription data indicating prescriptions written for the patient (including, dosages, amounts, and dates of each prescription, the prescribing physician, etc.), diagnosis data indicating respective diagnosis of the patient (e.g., for which ailments they are being treated), and/or suitable metadata (e.g., age of the patient, height of the patient, weight of the patient, sex of the patient, body fat percentage of the patient, and the like). It is noted that the list of items included in a patient record is provided for example only and may include additional or alternative types of data.

In embodiments, the insurer data store 234 may store insurer-related data. In embodiments, the insurer data store 234 stores an insurer database that stores and/or indexes insurer records. In embodiments, each insurer record may correspond to a respective insurance organization or other healthcare payor. An insurer record may include and/or may be related to an insurer ID that identifies the insurer, a set of clinic IDs that indicate healthcare organizations that the insurer is associated with, a set of patient IDs indicating patients that are customers of the insurer, and the like. It is noted that the list of items included in an insurer record are provided for example only and may include additional or alternative types of data.

In embodiments, the insurer data store 234 may include a claim database that stores and/or indexes claim records. Each claim record may correspond to an insurance-related event. An insurance related event may be any billable or non-billable occurrence that an insurance organization handles on behalf of an insured patient. In embodiments, a claim record may include and/or may be related to a claim ID that identifies the insurance-related event, a patient ID of the patient involved in the event, a clinic ID of the clinic that the patient visited, a physician ID of the provider who treated the patient, diagnosis data that indicates the provider's medical diagnosis, prescription data that indicates any prescriptions that were prescribed in relation to the event, including dosages, amounts, etc., lab test data that indicates any tests that were ordered in relation to the event including the results thereof, and associated metadata (e.g., date of the event on which the insurance-related event occurred). It is noted that the list of items included in a claim record is provided for example only and may include additional or alternative types of data.

In embodiments, the test results data store 238 may store test result-related data. In embodiments, the test results data store 238 stores a test results database that stores and/or indexes test result records. Each test result record may correspond to a respective lab test (e.g., genetic test, blood test, urine test, etc.). A test result record may include and/or be related to a test ID that identifies the test, a lab ID that identifies the testing lab that performed the test, a physician ID that identifies a physician that ordered the test, a test type that indicates the type of test, test result data that indicates the results of the test, an insurer ID that indicates an insurance organization of the patient that underwent the lab test, and any suitable metadata (e.g., a date of the test, an employee that processed the test, the machine that performed the test, etc.). It is noted that the list of items included in a test result record is provided for example only and may include additional or alternative types of data.

In embodiments, the data intake module 202 obtains data from one or more data sources (e.g., healthcare systems 130, pharmacy systems 140, testing lab systems 150 and/or insurance systems 160). The data intake module 202 may implement one or more public or private APIs (e.g., SOAP, RESTful APIs, and the like). The APIs may be passive (i.e., the data sources push data to the data intake module 202) and/or active (i.e., the data intake module 202 pulls data from the data sources).

In embodiments, the data intake module 202 includes an interception module that obtains prescription-related information relating to a clinic or physician. In some embodiments, the interception module may retrieve prescription-related data relating to prescriptions written by a physician or from a clinic. In some of these embodiments, the interception module may obtain prescription-related data from a prescription drug monitoring program (PDMP), whereby the interception module may obtain information relating to prescriptions of specific classes of medications written by respective physicians. In embodiments, the interception module may output any obtained prescription-related information to the data structuring module 204, including any metadata surrounding the prescription (e.g., the physician that wrote the prescription, the dosage amount, the clinic of the physician, the patient, the date of the prescription, the pharmacy at which the prescription is filled, etc.).

In embodiments, the data intake module 202 includes an interaction monitoring module that monitors communications between testing labs and customers (e.g., healthcare organizations, clinics, and/or physicians). In embodiments, the interaction monitoring module may determine each time a sales or service representative of a lab testing organization interacts with a customer (e.g., healthcare organizations, clinics, and/or physicians). In embodiments, the interaction monitoring module may also determine each time a customer orders a test from a lab. In these embodiments, the interaction monitoring module may output determined instances of interactions and/or orders to the data structuring module 204, including any metadata surrounding the interaction and/or order (e.g., the sales representative, the physician or representative of client that made an order, the date of the order, a patient corresponding to the order, etc.).

In embodiments, the data intake module 202 includes an insurer interface module. In embodiments, the insurer interface module interfaces with insurance systems 160. The insurer interface module may be configured to retrieve insurer-related information from an insurance system 160. The insurer-related information may include events related to a healthcare organization, clinic, and a physician. In some embodiments, the insurer interface module may retrieve physician-related information from the insured data store 1622. For example, the insurer interface module may request all claims and/or prescriptions that a physician, or a clinic thereof, billed to insurance company.

In embodiments, the data intake module 202 includes an EMR interface module that interfaces with healthcare systems 130. In embodiments, the EMR interface module may obtain physician-related data and/or patient-related data from the EMR data stores 132 of a healthcare system. For example, the EMR interface module may obtain prescriptions written by physicians, prescriptions written for patients, tests ordered by physicians and/or for patients, test results of patients, diagnoses of patients, patient metadata (e.g., age, sex, weight, body fat percentage), and/or any other suitable data. In embodiments, Fast Healthcare Interoperability Resources can be deployed to exchange resources and other data formats and elements through one or more application programming interfaces or suitable interfaces for exchanging electronic health records and/or electronic medical records. In embodiments, Fast Healthcare Interoperability Resources can be deployed or other suitable standard promulgated by the Health Level Seven International (HL7) health-care standards organization. In embodiments, Fast Healthcare Interoperability Resources can be deployed to build on or incorporate previous data format standards from HL7 and also deploy HTTP-based RESTful protocol, HTML and Cascading Style Sheets for user interface integration. In examples, discrete data elements can be accessed as services such that basic elements of healthcare like patients, admissions, diagnostic reports and medications can each be retrieved and manipulated via their own resource URLs or other suitable network connections. By way of these examples, the various protocols can deploy JSON, XML RDF and others for data representation. It will be appreciated in light of the disclosure that Fast Healthcare Interoperability Resources and other exchange resources can facilitate interoperation between legacy health care systems. This type of data exchange can also facilitate easier deployment of health care information to health care providers and individuals on various connected devices.

The data intake module 202 may obtain additional or alternative data relating to pharmacies, physicians, clinics, insurers, patients, lab testing facilities, and the like from any suitable data sources. Furthermore, it is noted that the data intake module 202, and the pharmacological tracking platform 100 as a whole, may be configured to conform to regulations concerning patient data privacy and personally identifiable information (PII). For example, the data intake module 202 may be configured to be HIPAA (Health Insurance Portability and Accountability Act) compliant.

In embodiments, the data structuring module 204 structures data collected by the data intake module 202. In embodiments, the data structuring module 204 structures the data into data records, such as clinic records, physician records, patient records, insurer records, and/or any other suitable types of records. The data structuring module 204 may create new records when necessary (e.g., when a new entity is detected), and can update preexisting records when a record corresponding to the collected data already exists (e.g., a previously detected entity). For example, if a physician that does not have a physician record corresponding thereto writes a new prescription, the data structuring module 204 may generate a new physician record based on the collected data. Likewise, when the physician writes a subsequent prescription, the data structuring module 204 may update the physician record of the physician based on the new prescription.

In embodiments, data structuring module 204 may generate physician profiles (e.g., physician records) based on the collected data. In some of these embodiments, the data structuring module 204 may use data obtained by the data intake module 202 to generate the physician profiles. In embodiments, a physician's name, one or more clinics that he or she operates out of, and other metadata may be obtained from a healthcare system 130 or insurance systems 160 associated with the physician. In embodiments, the data structuring module 204 may include prescription-related data obtained by the interception module to track a physician's history of writing prescriptions for controlled medications (e.g., opiates, benzodiazepines, amphetamines) in the physician profile. In embodiments, the data structuring module 204 may include ordering histories of the physician in the physician profile based on data obtained by the interaction monitoring module, the insurer interface module, and/or the EMR module. The data structuring module 204 may include other suitable data in the physician profile, including discipline events that indicate instances where the physician was previously disciplined. In embodiments, the data structuring module 204 may be configured to correlate the obtained data to the proper physician. For example, the data structuring module 204 may analyze the obtained data to identify various instances of data that match the physician's profile in order to properly attribute newly obtained data to the physician's profile.

In embodiments, the data structuring module 204 may generate patient profiles (e.g., patient records) based on the collected data. In some of these embodiments, the data structuring module 204 may use data obtained by the data intake module 202 to generate the patient profiles. In embodiments, a patient's name, clinics that the patient has been treated at, physicians that have seen the patient, and other metadata regarding the patient (e.g., age, sex, weight, height, body fat percentage, etc.) may be obtained from healthcare systems 130 or insurance systems 160 associated with the patient. In embodiments, the data structuring module 204 may include prescription-related data obtained by the interception module to track the prescriptions of controlled medications (e.g., opiates, benzodiazepines, amphetamines) written for the patient in the patient profile. This information may include the medication that was prescribed, the physician who wrote each prescription, when the prescription was written, the pharmacy that filled the prescription, and other relevant prescription-related data items. In embodiments, the data structuring module 204 may include an ordering history of the patient in the patient profile based on data obtained by the interaction monitoring module, the insurer interface module, and/or the EMR module. The patient profile may indicate which tests were ordered, the physician that ordered the tests, when the tests were ordered, and the results of the tests. In embodiments, the data structuring module 204 may be configured to correlate the obtained data to the proper patient. For example, the data structuring module 204 may analyze the obtained data to identify various instances of data that match the patient's profile in order to properly attribute newly obtained data to the patient's profile.

In embodiments, the reporting module 206 is configured to report notifications and/or recommendations to third parties. Third parties may include healthcare organizations (e.g., hospitals, clinics), pharmacies, testing labs, and/or insurance organizations. Examples of notifications may include a notification that a patient is likely misusing a prescription medication, a notification that a patient has had superfluous tests performed on them, a notification that a physician is prescribing too many controlled medications, a notification that a physician is over ordering lab tests, and the like. Examples of recommendations may include recommendations to order tests for a patient prior to or after a prescribed treatment. In some embodiments, the reporting module 206 reports notifications on behalf of a user (e.g., a sales or service representative of a testing lab). In these embodiments, the reporting module 206 may present the recommendation or notification to a user, whereby the user may select to send the notification or recommendation. In embodiments, the notification module 206 reports notifications and/or recommendations to third parties automatically. In these embodiments, a third party (e.g., healthcare organization or insurer) may request such information from the pharmacological tracking platform 100 and/or a user may elect to have notifications and/or recommendations sent automatically.

In embodiments, the client interfacing module 208 provides an interface for users to contact, message, and communicate with third parties (e.g., customers and potential customers). In some embodiments, the client interfacing module 208 provides a mechanism by which a healthcare supplier (e.g., a lab test facility) may conduct business with its customers and/or potential customers. The client interfacing module 208 may provide a graphical user interface that allows a user to draft messages, set workflows, and share information with third parties.

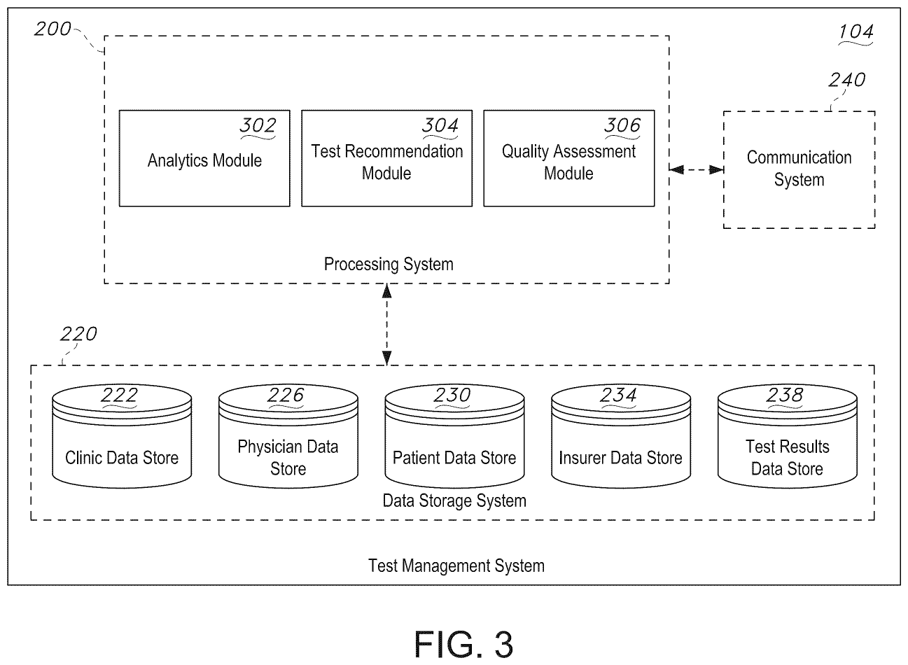

FIG. 3 illustrates an example of a test management system 104 according to some embodiments of the present disclosure. In embodiments, the test management system 104 may include a processing system 200, a data storage system 220, and a communication system 240. In some embodiments, a test management system 104 shares these same hardware resources (e.g., executed by the same set of server devices) as the CRM system 102. In some embodiments, a test management system 104 does not share the hardware resources with the CRM system 102 and may be hosted on an independent computing environment that communicates over a public network via one or more APIs.

In embodiments, the processing system 200 may execute an analytics module 302, a testing recommendation module 304, and/or a quality assessment module 306. The processing system 200 may execute additional or alternative modules without departing from the scope of the disclosure. In embodiments, the data storage system 220 may store a clinic data store 222, a physician data store 226, a patient data store 230, an insurer data store 234, and a test results data store 238, as was described with respect to the CRM system 102.

In embodiments, the analytics module 302 performs various analytics tasks relating to ordered lab tests. For example, the analytics module 302 may perform predictive and/or descriptive data analytics on a physician's or clinic's ordering history of lab tests. Such analytics may uncover unnecessary ordering of tests or even fraudulent ordering patterns. In another example, these types of data analytics may identify physicians or clinics that are potentially underutilizing lab tests in their practices.

In embodiments, the analytics module 302 analyzes physician profiles to determine if a physician is over-ordering or under-ordering lab tests. In some of these embodiments, the analytics module 302 performs statistical analytics techniques on a set of physician profiles to determine whether a physician is over-ordering or under-ordering lab tests. In some of these embodiments, the physician records may be clustered using K-nearest neighbors or K-means clustering to identify physician records that appear in the same cluster(s) as physician records that have been deemed to be anomalous (i.e., under-ordering or over-ordering). In these embodiments, the analytics module 302 may cluster the records based on a set of features, which may include the amount of patients seen, the amount of tests ordered during a time period, the types of tests ordered, the amount charged to insurance companies and/or the patient to run the tests, the diagnosis of the patients, and/or other suitable features. As most physicians having the same or similar specialties, they will have consistent ordering histories, physicians deviating from the normal patterns may be identified based on the clusters to which they belong. The analytics module 302 may perform other types of statistical analysis on physician records, such as deep learning and statistical regressions, amongst others.

In embodiments, the analytics module 302 may analyze the ordering practices of clinics as well. In these embodiments, the analytics module 302 (or another suitable module of the platform 100) may group physician records together based on which clinic they operate from (or primary clinic they operate from) to obtain a clinic profile. In embodiments, the analytics module 302 may then perform statistical analysis on the clinic profiles to identify clinics that are collectively over-ordering or under-ordering lab tests (e.g., as described with respect to physician profiles). Furthermore, if a clinic is found to have a statistical anomaly, the physician records corresponding to the individual physicians of the clinic may be analyzed individually to ensure that the anomaly is not attributable to a single or small set of physicians.

Upon determining that a physician and/or a clinic exhibits an anomalous ordering history, the analytics module 302 may output a notification indicating the anomaly. In some embodiments, the analytics module 302 may output the notification to the reporting module 206 of the CRM platform 102. In other embodiments, the analytics module 302 may output notifications directly to third parties, such as insurance companies or regulatory agencies.

In embodiments, the testing recommendation module 304 recommends lab tests for patients. In some embodiments, the testing recommendation module 304 recommends one or more tests for patients given a prescribed treatment, so as to improve the likelihood that the treatment is effective to the patient. In these embodiments, the testing recommendation module 304 may receive a proposed prescription for a patient from an external system (e.g., a healthcare system or healthcare organization of a patient being prescribed or an insurance system of an insurer of the patient) or from the CRM system 102. In embodiments, the proposed prescription may indicate the patient being prescribed and/or may include information relating to the patient. In the former scenario, the testing recommendation module 304 may retrieve the relevant information relating to the patient from the patient datastore 230 based on an identifier of the patient.

In response to the proposed prescription and the patient information, the testing recommendation module 304 may determine whether to recommend preliminary lab tests before the patient undergoes the prescribed treatment based on the proposed prescription and information relating to the patient. In embodiments, a recommendation may indicate one or more tests that are recommended for the patient given the proposed prescription.

In embodiments, the testing recommendation module 304 may employ a machine learning approach to determine whether to recommend one or more preliminary lab tests. In some of these embodiments, the testing recommendation module 304 may generate a set of features (e.g., a feature vector) based on the proposed prescription (e.g., a medication identifier, a dosage amount, a number of pills, etc.) for the patient and the patient information (e.g., a patient's age, a patient's sex, a patient's weight, a patient's body type, a patient's medication history). In these embodiments, the testing recommendation module 304 may input these features into one or more machine learned models that determine whether a particular test (e.g., a genetic test, a blood test, etc.) or set of tests should be performed. The machine learned model may be any suitable type of machine learned models, such as a neural network, a deep neural network, a recurrent neural network, a Hidden Markov Model, a regression based model, a decision tree (e.g., a classification tree), and the like. In some embodiments, different machine learned models may be trained for different types of medication or classes of medications. In this way, the testing recommendation module 304 may select one or more models to leverage based on the type of medication in the potential prescription.

In some embodiments, a machine learned model may correspond to a specific type of test (e.g., a genetic test, a blood test, or a urine analysis test) and may output a recommendation as to whether the specific type of test should be performed. In these embodiments, the test management system 104 may leverage multiple models, such that each respective model may correspond to a different type of test and may output a recommendation corresponding to the respective type of test. In embodiments, the recommendation may include a confidence score that indicates a degree of confidence in the recommendation. The testing recommendation module 304 may determine whether to select a recommendation made by a model based on the confidence score corresponding to the recommendation (e.g., when the confidence score exceeds a threshold).

In some embodiments, a machine learned model may be trained to recommend multiple types of tests. In these embodiments, the testing recommendation module 304 may input the set of features to the model, which outputs a confidence score for each type of potential test given the features (e.g., the features extracted from the proposed prescription and the patient information). In response, the testing recommendation module 304 may select one or more tests based on the respective confidence scores thereof (e.g., any test having a confidence score greater than a threshold).

In embodiments, the test management system 104 may be configured to employ a rules-based approach to determine whether any tests should be performed on a patient given a prescribed medication. In these embodiments, test management system 104 may assess the patient with a set of rules that are determined for a respective medication. The conditions which trigger certain rules may be learned using analytics that are derived from outcomes pertaining to the medication. For example, a certain medication may be ineffective for people over the age of sixty having a certain genetic characteristic, but effective for all other segments of the population. In this example, if the patient is over the age of sixty, the test management system 104 may determine that the patient should undergo genetic testing to determine whether the patient exhibits the certain genetic characteristic.

Upon determining one or more recommended tests for the patient given the proposed prescription, the testing recommendation module 304 may output a recommendation that includes a respective test type indicator for each respective test that is being recommended. In some embodiments, the testing recommendation module 304 may output the recommendation to the reporting module 206 of the CRM system 102. In other embodiments, the testing recommendation module 304 may output recommendations directly to third parties, such as healthcare providers (e.g., physicians, nurses, etc.). In embodiments, a computing device associated with one or more third parties may provide outcome data, indicating whether the test was performed, the prescription that was ultimately prescribed to the patient, and whether the prescription was effective in treating the patient. For example, this information may be obtained from the healthcare system 130 and/or the insurance system 160. In embodiments, this outcome data may be used to reinforce the models that are used to make the recommendation.

In some embodiments, the testing recommendation module 304 recommends one or more tests for patients undergoing or having undergone a treatment, so as to improve the post-treatment monitoring of the patient.

In embodiments, the quality assessment module 306 measures the quality and/or effectiveness of respective lab testing organizations. The quality assessment module 306 may utilize machine learning, statistical analysis, and/or rules-based analysis to assess the quality of a lab testing organization.

In embodiments, the quality assessment module 306 may generate a lab quality profile of a testing lab. In some of these embodiments, the quality assessment module 306 may aggregate transaction histories of a collection of testing lab organizations (e.g., order histories of respective testing labs) over a period of time. The quality assessment module 306 may analyze the volumes and types of the tests indicated in each respective transaction history, and may compile data sets relating to pre-analytical, analytical, and/or post-analytical laboratory issues. The quality assessment module 306 may receive and parse human-input information relating to each laboratory issue. In some embodiments, the quality assessment module 306 may combine differently worded descriptions that have similar meanings or the same meaning. The quality assessment module 306 may then automatically generate plain-language textual summaries (e.g., using natural language generation) corresponding to the testing lab, where the summaries are grouped by aggregated laboratory issues.

In embodiments, the quality assessment module 306 is configured to generate a lab quality profile of a testing lab that includes a textual summary of the testing lab. In some of these embodiments, the quality assessment module 306 may aggregate transaction histories of a collection of testing lab organizations (e.g., order histories of respective testing labs) over a period of time. The quality assessment module 306 may analyze the volumes and types of the tests indicated in each respective transaction history, and may compile data sets relating to pre-analytical, analytical, and/or post-analytical laboratory issues. The quality assessment module 306 may receive and parse human-input information relating to each laboratory issue. In some embodiments, the quality assessment module 306 may combine differently worded descriptions that have similar meanings or the same meaning. The quality assessment module 306 may then automatically generate plain-language textual summaries (e.g., using natural language generation) corresponding to the testing lab, where the summaries are grouped by aggregated laboratory issues.

In embodiments, the quality assessment module 306 is configured to generate an improvement plan for a testing lab. In some of these embodiments, the quality assessment module 306 may aggregate transaction histories of a collection of testing lab organizations (e.g., order histories of respective testing labs) over a period of time. The quality assessment module 306 may analyze the volumes and types of the tests indicated in each respective transaction history, and may compile data sets relating to pre-analytical, analytical, and/or post-analytical laboratory issues. The quality assessment module 306 may receive and parse human-input information relating to each laboratory issue. In some embodiments, the quality assessment module 306 may combine differently worded descriptions that have similar meanings or the same meaning. The quality assessment module 306 may then automatically generate a plain-language textual improvement plan (e.g., using natural language generation) corresponding to the testing lab based on the identified laboratory issues.

In embodiments, the quality assessment module 306 is configured to identify a primary cause of an identified lab issue. In some of these embodiments, the quality assessment module 306 may aggregate transaction histories of a collection of testing lab organizations (e.g., order histories of respective testing labs) over a period of time. The quality assessment module 306 may analyze the volumes and types of the tests indicated in each respective transaction history, and may compile data sets relating to pre-analytical, analytical, and/or post-analytical laboratory issues. The quality assessment module 306 may receive and parse human-input information relating to each laboratory issue. In some embodiments, the quality assessment module 306 may combine differently worded descriptions that have similar meanings or the same meaning. The quality assessment module 306 may then map the identified issues to an ontology that includes different types of entities that relate to the platform 100 (e.g., testing labs, physicians, clinics, etc.). The quality assessment module 306 may then automatically generate an indication of the most likely entity that was the cause of each respective issue.

In embodiments, the quality assessment module 306 is configured to generate statistics relating to the employees of the testing lab. In some of these embodiments, the quality assessment module 306 may aggregate transaction histories of a collection of testing lab organizations (e.g., order histories of respective testing labs) over a period of time. The quality assessment module 306 may analyze the volumes and types of the tests indicated in each respective transaction history, and may compile data sets relating to pre-analytical, analytical, and/or post-analytical laboratory issues, test speeds, and/or performance metrics. The quality assessment module 306 may then compile time utilization and/or workload statistics for each testing lab and/or the lab workers employed therein. In some embodiments, the quality assessment module 306 may activate a workflow that identifies a source of a respective test that proceeded a drop in productivity and activates a quality review of the source. Additionally or alternatively, the quality assessment module 306 may activate a workflow that identifies one or more pieces of equipment used during a test that resulted in an issue, and activates a quality review of the one or more pieces of equipment.

The test management system 104 may include additional or alternative components that perform various tasks related to the oversight and/or management of lab tests.

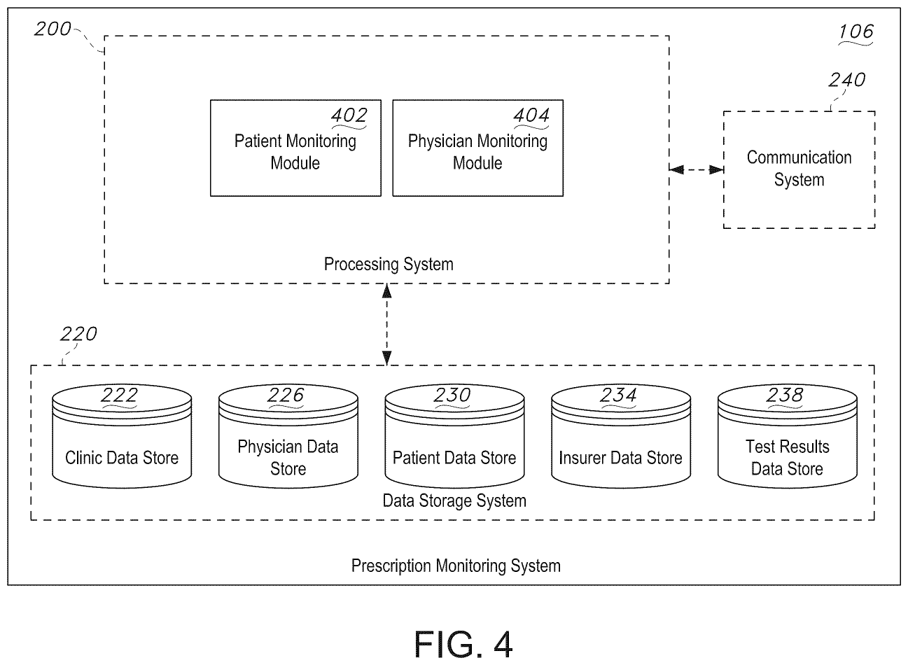

FIG. 4 illustrates an example of a prescription monitoring system 106 according to some embodiments of the present disclosure. In embodiments, the prescription monitoring system 106 may include a processing system 200, a data storage system 220, and a communication system 240. In some embodiments, a test management system 104 shares these same hardware resources (e.g., executed by the same set of server devices) as a CRM system 102 and/or a test management system 104. In some embodiments, a prescription monitoring system 106 does not share the hardware resources with a CRM system 102 and/or a test management system 104, and may be hosted on an independent computing environment.

In embodiments, the processing system 200 may execute a patient monitoring module 402 and/or a physician monitoring module 404. The processing system 200 may execute additional or alternative modules without departing from the scope of the disclosure. In embodiments, the data storage system 220 may store a clinic data store 222, a physician data store 226, a patient data store 230, an insurer data store 234, and a test results data store 238, as was described with respect to the CRM system 102.

In embodiments, the patient monitoring module 402 monitors test results associated with patients that are prescribed controlled medications, such as opiates, amphetamines, and/or benzodiazepines to determine whether a patient is misusing the prescribed medication. In these embodiments, the patient monitoring module 402 may begin monitoring a patient's use of a controlled medication when the patient is initially prescribed the controlled medication. The patient monitoring module 402 may determine a patient is initially prescribed a controlled medication from, for example, a PDMP, a healthcare system 130, and/or an insurance system 160. In certain scenarios, the treating physician may require the patient to undergo testing (e.g., urine analysis or blood testing) while prescribed the medication. In embodiments, the results of the test corresponding to a patient may be stored in the test results data store 238, and may be associated with the patient record of the patient. Each time new test results are received for a patient, the patient monitoring module 402 may analyze the test results to determine if the patient is likely misusing the medication. As previously discussed, misusing a medication may refer to overusing/abusing the medication and/or underusing the medication.

In some embodiments, the patient monitoring module 402 may generate a usage profile of the patient based on the collection of the prescriptions of the controlled medication that have been written for the patient, a collection of test results corresponding to the patient during the time period where the patient was taking the medication, and patient information of the patient (e.g., patient's age, weight, body type, sex, activity rate). In these embodiments, the patient monitoring module 402 may determine if a patient is misusing the controlled medication based on the usage history of the patient. The patient monitoring module 402 may identify misuse in any suitable manner.

In embodiments, the patient monitoring module 402 may employ machine learned classification models that are trained to classify misuse of controlled medications. In some of these embodiments, the machine learning system 108 may train the machine learned classifications on training data sets that include usage profiles of patients that were deemed to be using a controlled medication properly and patients that were deemed to be misusing the controlled medication (e.g., usage profiles of patients) that were deemed to be underusing the medication (which may suggest selling of medication or no need for the medication) and/or usage profiles of patients that were deemed to be overusing/abusing the medication. In embodiments, the patient monitoring module 402 may generate a set of features (e.g., a feature vector) from the usage profile of the patient being monitored and may input the set of features into the classification model. The classification model may output a classification corresponding to the patient that indicates whether there is potential misuse detected, and in some of these embodiments, a type of misuse (e.g., overuse/abuse or underuse). In embodiments, the classification may include a confidence score in the classification, whereby the patient monitoring module 402 may select a classification based on the confidence score thereof (e.g., when the confidence score exceeds a threshold). In the event the patient monitoring module 402 determines that the classification indicates misuse, the patient monitoring module 402 may output a notification indicating the potential misuse (which may also include the type of misuse).

In embodiments, the patient monitoring module 402 may perform analytics to identify potential misuse. In some of these embodiments, the patient monitoring module 402 may perform a statistical analysis on the usage profile to determine trends in the patient's dosage amounts and test results that would indicate overuse or underuse. In some embodiments, the patient monitoring module 402 may analyze the usage profile of the patient in relation to the usage profiles of other patients, including patients deemed to be misusing the medication that the patient is prescribed and patients deemed to be using the medication properly. In some of these embodiments, the patient monitoring module 402 may implement a clustering technique (e.g., K-nearest neighbors or K-means clustering) to determine whether the patient belongs to a cluster where the usage profiles indicated misuse or to a cluster where the usage profiles indicated proper use. In the event the usage profile of the patient is clustered to a cluster where the usage profiles indicated misuse, the patient monitoring module 402 may determine that the patient is likely misusing the medication and may output a notification indicating the potential misuse (which may also include the type of misuse).

The patient monitoring module 402 may monitor patients for potential misuse of a medication in other suitable manners. For example, the patient monitoring module 402 may implement a rules-based approach to identify potential misuse. Upon determining that a patient is likely misusing a controlled medication, the patient monitoring module 402 may output a notification indicating that the patient is likely misusing the controlled medication (which may also indicate the type of misuse). In some embodiments, the patient monitoring module 402 may output the notification to the reporting module 206 of the CRM system 102. In other embodiments, the patient monitoring module 402 may output notifications directly to third parties, such as the treating physician of the patient.

In embodiments, the physician monitoring module 404 monitors a physician's prescribing history and/or the lab tests of the physician's patients to determine if a physician is likely overprescribing controlled medications. In some of these embodiments, the physician monitoring module 404 may generate a prescribing profile for any physician that prescribes controlled medications. In embodiments, the prescribing profile may indicate each instance that the physician prescribed a controlled medication and, in some of these embodiments, the diagnosis leading to the prescription. In some of these embodiments, the prescribing profile may also indicate test results of patients of the physician that were prescribed the controlled medication and/or the determinations made by the patient monitoring module 402 as to whether the patient was misusing the medication or properly using the medication. In these embodiments, a physician who has higher ratios of patients who are likely misusing the medication versus patients that are likely using the medication properly may be more likely to be flagged as overprescribing the medication.

In embodiments, the physician monitoring module 404 may determine whether a physician is likely overprescribing a controlled medication based on the prescribing profile of the physician. The physician monitoring module 404 may implement any suitable technique to determine whether the physician is likely overprescribing a controlled medication. In embodiments, the physician monitoring module 404 may implement machine learning techniques to determine whether the physician is likely overprescribing a controlled medication. For example, the physician monitoring module 404 may generate a set of features based on the physician's prescribing profile and may input the set of features into a machine learned classification model that is trained to identify instances where a physician is likely overprescribing a controlled medication. In some of these embodiments, the machine learned classification model may be trained on training data sets that include prescribing profiles where the respective physician was deemed to be overprescribing controlled medications and prescribing profiles where the respective physician was deemed not to be overprescribing (e.g., the physician's practices were deemed normal or less than normal).

In embodiments, the physician monitoring module 404 may implement statistical analysis to determine whether the physician is likely overprescribing a controlled medication. In some of these embodiments, the physician monitoring module 404 may cluster (e.g., K-nearest neighbors or K-means clustering) prescribing profiles of physicians to determine whether the physician being monitored is likely overprescribing the medication. The physician monitoring module 404 may use additional or alternative statistical analysis techniques to determine whether the physician is likely overprescribing a controlled medication. In some embodiments, the physician monitoring module 404 may implement a rules-based approach to determine whether the physician is likely overprescribing a controlled medication.

Upon determining that a physician is likely overprescribing a controlled medication, the physician monitoring module 404 may output a notification indicating that the physician is likely overprescribing a controlled medication. In some embodiments, the physician monitoring module 404 may output the notification to the reporting module 206 of the CRM system 102. In other embodiments, the physician monitoring module 404 may output notifications directly to third parties, such as insurance companies or regulatory agencies.

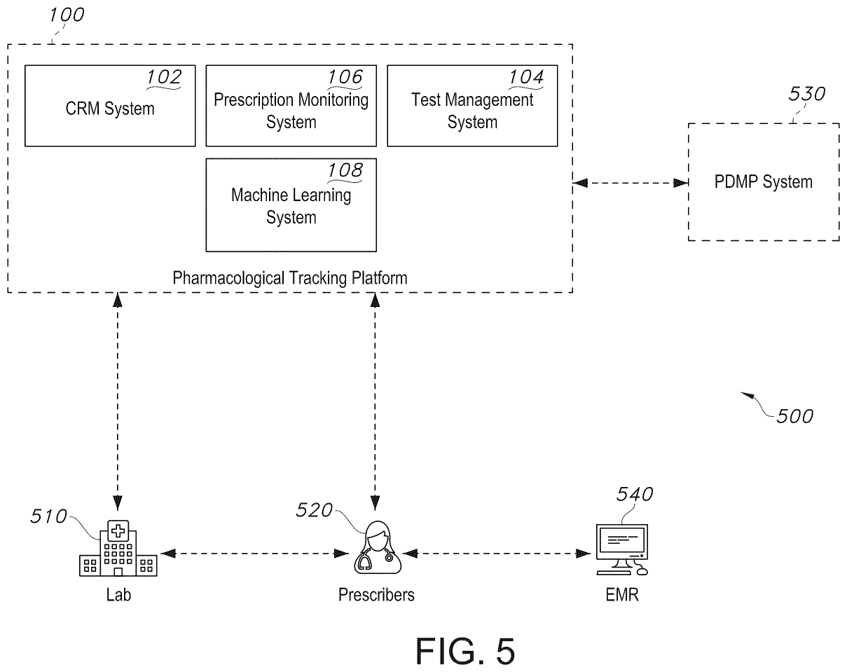

Various example implementations of the report generation and outputting function of the pharmacological tracking platform 100 will be described in reference to FIG. 5-FIG. 10. As mentioned above, the present disclosure provides for generating an enhanced toxicology report corresponding to a patient that is a simple and easy to understand summary of the use and potential misuse of controlled substances for a patient. An example reporting system environment 500 can include the pharmacological tracking platform 100, a laboratory or laboratory system 510 (referred to herein as “laboratory 510”), a user or user system 520 (referred to herein as “user 520”), a PDMP or PDMP system 530 (referred to herein as “PDMP 530”), and an EMR or EMR system or database 540 (referred to herein as “EMR 540”). Each of the pharmacological tracking platform 100, the laboratory 510, the user 520, the PDMP 530, the EMR 540 can comprise one or computing devices operating to perform the techniques described herein. Further, the pharmacological tracking platform 100, the laboratory 510, the user 520, the PDMP 530, the EMR 540 can be in communication with any of the other components of the environment 500, for example, via a network (such as network 380). In some aspects, the pharmacological tracking platform 100, the laboratory 510, the user 520, the PDMP 530, and the EMR 540 can share various information, assessments, calculations, records, determinations, etc. (as described below) to assist in the creation, storage, and maintenance of an enhanced toxicology report 600, which is described more fully below.

The pharmacological tracking platform 100 can receive laboratory test results from the laboratory 510, e.g., based on an order from the user 520 for a toxicology screen of a patient. The laboratory test results received by the pharmacological tracking platform 100 can correspond to the patient and be indicative of the toxicology screen of the patient. The toxicology screen can be one or more of the various known drug tests that determine the type and approximate amount of certain drugs and medications that the patient has taken. In certain circumstances, the user 520 will be a healthcare care professional (a doctor, a nurse, a dentist, a physician's assistant, or the like) that has ordered the toxicology screen to assist in the treatment of the patient, although other possibilities are within the scope of the present disclosure. The laboratory 510 can proactively provide the laboratory test results to the pharmacological tracking platform 100. Alternatively, the pharmacological tracking platform 100 can request the laboratory test results from the laboratory 510, e.g., upon being notified by the user 520 of the toxicology screen.

The pharmacological tracking platform 100 can further retrieve controlled substance prescription data for the patient from the PDMP 530. The controlled substance prescription data can include prescriptions of controlled substances issued to the patient for a relevant time period (e.g., the previous two or more years). In some aspects, the pharmacological tracking platform 100 can retrieve the controlled substance prescription data upon being notified by the user 520 of the toxicology screen and/or upon receiving the laboratory test results for the patient from the laboratory 510.

The pharmacological tracking platform 100 can analyze the controlled substance prescription data and the laboratory test results to determine various factors, measurements, calculations, etc. relating to the use and potential misuse of controlled substances for the patient. For example only, the pharmacological tracking platform 100 can determine a daily morphine milligram equivalent of the patient for a given time period, an overdose risk score, and a drug consistency assessment. The daily morphine milligram equivalent of the patient for the given time period can correspond to a cumulative intake of opioid class drugs by the patient on a daily basis for the given time period. The overdose risk score can be a number, grade, or other scoring indexes that are indicative of a likelihood of an unintentional overdose by the patient, as further described below. The drug consistency assessment is representative of a match between the controlled substance prescription data and the laboratory test results for the patient.

In certain aspects, the pharmacological tracking platform 100 can also or alternatively obtain patient attributes of the patient from one or more patient data sources (e.g., such as a data storage system 220). Examples of such patient attributes can correspond to an age of the patient, a weight of the patient, a body type of the patient, an activity level of the patient, and a diagnosis of the patient, although this is not an exhaustive list of patient attributes. In such implementations, the enhanced toxicology report can be further based on any or any combination of these patient attributes, which may assist the pharmacological tracking platform 100 in more accurately determining the various indications relating to the use and potential misuse of controlled substances for the patient.

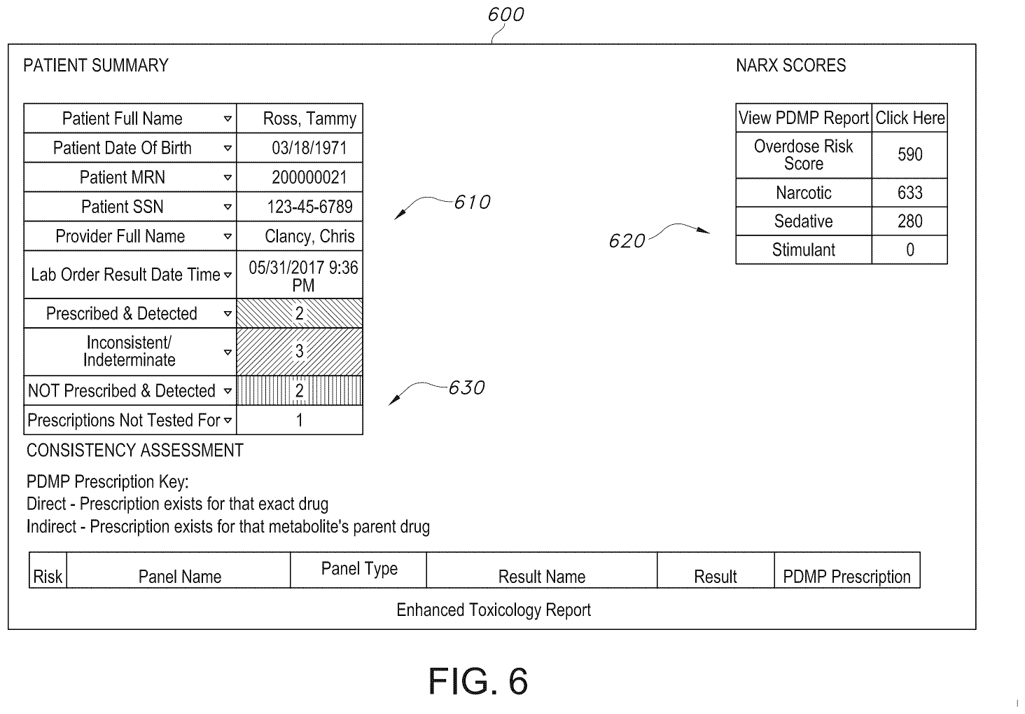

Based on the determined factors relating to the use and potential misuse of controlled substances for the patient (e.g., the daily morphine milligram equivalent of the patient for a given time period, the overdose risk score, and the drug consistency assessment), the pharmacological tracking platform 100 can generate an enhanced toxicology report corresponding to the patient. An example of such an enhanced toxicology report 600 is illustrated in FIG. 6-FIG. 10. While each of FIG. 6-FIG. 10 shows a specific view or presentation of the information in the enhanced toxicology report 600, it should be appreciated that the enhanced toxicology report 600 can include more or less information depending on the specific implementation of the pharmacological tracking platform 100.

Referring now to FIG. 6, a first view of the example enhanced toxicology report 600 is shown. The example enhanced toxicology report 600 can include patient identification information 610, such as the patient name, date of birth, medical record number, and/or social security number. Further, the example enhanced toxicology report 600 can include one or more of the determined overdose risk scores 620. As shown in FIG. 6, the illustrated overdose risk scores 620 include individual overdose risk scores for various drug types (e.g., narcotics, sedatives, stimulants), as well as an overall overdose risk score that is independent of drug type. As more fully described below, the enhanced toxicology report 600 shown in FIG. 6 can also include a drug consistency assessment 630 that is representative of a match between the controlled substance prescription data and the laboratory test results for the patient.

The drug consistency assessment 630 shown in FIG. 6 includes multiple drug consistency scores based on the drug consistency assessment, wherein each particular drug consistency score is indicative of a match between a particular drug identified in either or both of the controlled substance prescription data and the laboratory test results for the patient. For example only, a particular drug consistency score can indicate one of the following circumstances: (i) a prescribed and detected condition in which the particular drug is identified in both of the controlled substance prescription data and the laboratory test results for the patient; (ii) a detected but not prescribed condition in which the particular drug is identified in the laboratory test results for the patient but not the controlled substance prescription data; (iii) an inconsistent condition in which (a) the particular drug is a drug metabolite of a parent drug and is identified in the laboratory test results for the patient and the controlled substance prescription data indicates a prescription for the parent drug, or (b) the particular drug is identified in the controlled substance prescription data and the laboratory test results for the patient indicate that the particular drug is not present at a prescribed amount in the patient; and (iv) a particular drug is identified in the controlled substance prescription data but no corresponding laboratory test was ordered to detect the particular drug. A recommendation to the user could be made to order a lab test to check for the presence of the particular drug. As shown in FIG. 6, each of these conditions can be separately indicated, e.g., by a number and/or by a color or other indication. For example only, in some implementations, the drug consistency assessment 630 can also include a confidence score indicative of a confidence level in the determined assessment of drug consistency.

In the various examples herein, PDMPs can be state-run programs that collect and/or distribute data about the prescription and dispensation of federally controlled substances. In some implementations, PDMPs are electronic databases that allow healthcare providers to see patients' prescription histories, thereby allowing doctors and other drug prescribers to check whether a patient has been prescribed and dispensed controlled drugs, such as opioids, before prescribing others to the patient. Some PDMPs also track non-fatal and fatal opioid overdoses, identify risk factors for fatal overdoses in patients, and track toxicology testing. The US federal government provides funding to the states so that each state can fund its PDMP program.

The goal of PDMPs is to help to prevent adverse drug-related events through opioid overdoses, drug diversion, and/or substance abuse by decreasing the amount and/or frequency of opioid prescribing. Such PDMPs may be accessed and utilized by physicians, physician assistants, nurse practitioners, dentists, and/or other prescribers, pharmacists, and/or pharmacy support staff, as well as law-enforcement agencies and research agencies. These parties may act individually or collaborate together to support the legitimate medical use of controlled substances, while limiting their abuse and/or diversion, as further described herein.

Pharmacies and dispensing prescribers of controlled substances may be required to register with their respective state PDMPs and/or to report the dispensation of such prescriptions to an associated electronic online database. For example only, when a pharmacist dispenses drugs to a patient or is about to dispense drugs to a patient, the pharmacy logs the dispensation with the PDMP. In some states, pharmacies are required to log drug dispensation with the PDMP in real-time or substantially real-time. In other states, pharmacies log drug dispensation daily, weekly, monthly, or at some other interval. Once dispensation of a drug has been logged, a record of the dispensation is accessible by one or more of doctors, other healthcare providers, state insurance programs, healthcare licensure boards, state health departments, and first responders and other law enforcement personnel. In some cases, PDMP information is shared between states, and/or is used by the federal government, such as to improve statistical gathering and legislation to combat opioid abuse.

As briefly mentioned above, PDMP information can be used by doctors and other providers of prescriptions to help prevent patients from seeing different doctors to receive redundant drug prescriptions from each of the doctors, which is sometimes referred to as “doctor shopping.” If a doctor views PDMP prescription logs for a patient before prescribing an opioid to the patient, the doctor may see that the patient has already been prescribed one or more opioids recently by other doctors and may take the appropriate action, e.g., refusing to provide one or more additional opioid prescriptions to the patient. By reducing such “doctor shopping,” PDMPs can assist in curbing opioid addiction.

PDMP information can also be used by lawmakers and administrative agencies to assist in drafting legislation to curb opioid addiction. The lawmakers and administrative agencies can use PDMP log information to inform themselves about general opioid prescribing practices in states, regions, or other geographical areas. The lawmakers and administrative agencies can then pass legislation and regulations using real data about prescribing practices to accurately target trends and issues with the current healthcare system in the geographical area(s) under consideration. For example only, state lawmakers and administrative agencies can access PDMP logs to acquire data regarding opioid prescribing practices within their state, and federal lawmakers and administrative agencies can access PDMP logs of multiple states to acquire data regarding opioid prescribing practices and trends between states and in the whole United States.

In some aspects, PDMP information can also be used by law enforcement agencies and first responders to assist in handling cases of opioid addiction, overdose, and withdrawal. For example only, the law enforcement agencies and first responders can check PDMP logs for an individual who is addicted to opioids or is experiencing opioid overdose or withdrawal to accurately ascertain an extent of the individual's opioid use and thereby provide proper assistance, such as by providing drugs that block the effects of opioids, e.g., Naloxone.

In yet another use case, PDMP information can be used by healthcare personnel (such as anesthesiologists and nurses) to assist in medical procedures that do not generally involve opioids. For example, prior to a surgical procedure, a doctor, nurse, or anesthesiologist can check PDMP logs to determine whether a patient is currently taking opioids. The doctor, nurse, or anesthesiologist can then more accurately prepare the patient for surgery, such as by raising or lowering levels of anesthetic used during the surgery to account for interactions between opioids and anesthesia. In some cases, patients may be reluctant to disclose opioid use or addiction to healthcare personnel due to stigma, embarrassment, personal issues, or other reasons. In such cases, it may be important for the healthcare personnel to determine an extent of opioid use by the patient in order to foresee complications regarding the interactions between opioids and anesthesia or other drugs used during care.

While the above discussion of PDMPs has been limited to PDMPs as implemented in the United States, it should be appreciated that similar programs exist in many other countries and regions, some of which are described below. For example only, several European countries have implemented national drug prescription tracking and information sharing. In France, the National Agency for the Safety of Medicines and Health Products (ANSM) develops several activities both in France and on behalf of the European Union to track prescribing practices and help develop strategies for curbing opioid abuse, such as regulation of prescription and dispensing conditions and reductions in prescription periods. The ANSM has an online reporting tool for use by healthcare professionals, pharmacists, and patients to report use and overuse of opioids, methods of use of opioids, prescribing practices of opioids, and compliance or noncompliance with the laws and regulations regarding opioids. Spain and Germany have similar national systems for providing information, conducting research, and receiving incident reports regarding opioid drugs and abuse thereof.

Internationally, the European Union (EU) drug agency in Lisbon (European Monitoring Centre for Drugs and Drug Addiction) has established the EU4MD database to track prescription drug importation and exportation to and from countries neighboring the EU, known as “neighborhood countries.” The neighborhood countries include Belarus, Ukraine, Moldova, Georgia, Armenia, Azerbaijan, Lebanon, Israel, Palestine, Jordan, Egypt, Libya, Tunisia, Algeria, and Morocco. The EU4MD seeks to establish a better understanding of drug markets, capacity for development for forensic analysis, assessment of the environmental impact of drug production, identification of drug problem “hot spots,” mapping of production and trafficking dynamics, technological innovations, threat assessment, and responses to emerging issues to support the EU and neighboring countries.

In Denmark, the Register of Medicinal Product Statistics includes data on all drugs sold in primary care or purchased for use in Danish hospitals. Aggregate data on gross sales of drugs are freely available, and individual-level data on prescriptions filled by Danish residents at community pharmacies are available as an independent sub-registry known as the National Prescription Registry. However, the National Prescription Registry does not provide information regarding drugs used during hospital admissions, drugs used by certain institutionalized individuals, such as individuals institutionalized with psychiatric illness, and drugs supplied directly by hospitals or treatment centers.

In Finland, a service called Kanta allows healthcare personnel and pharmacies to record information regarding drug prescribing. The records are available to patients and can be made publicly available with patient consent. Prescriptions issued by healthcare professionals in Finland are accessible in Kanta and are recorded for at least twenty years. Information regarding deceased individuals is available for up to twelve years after the patient's death. Information stored by Kanta is available to pharmacies and healthcare providers in Finland, and in some cases is also accessible in other European countries.

The Norwegian Prescription Database (NorPD) monitors drugs dispensed by prescription in Norway. NorPD collects and processes data on drug consumption in Norway to map usage trends and monitor trends over time, and can be used as a resource for research, as well as to give health authorities a statistical management tool for quality control of drug use and to give prescribers a basis for internal control and quality improvement of their prescribing practices. NorPD data sorted by demographic, such as sex, age, or region, is publicly available, but information about a patient's name, address, or national identification is not stored.

In Sweden, the Swedish Prescribed Drug Register (SPDR) contains information about age, sex, and unique identifier of each patient to whom a drug has been prescribed. The SPDR also includes information regarding drug names, costs, the professional and training of the prescriber, the prescribed amount of drug, the date of prescription, the date of collection, and other similar information. The information stored by the SPDR is available to researchers, journalists, city council investigators and authorities, and pharmaceutical industry representatives. Personal information such as patient name and identifier are private.

In China, the China Food and Drug Administration has implemented the Chinese Electronic Drug Monitoring Network (CEDMN) to track prescription drug products. Information is tracked and exchanged in the CEDMN via XML data. CEDMN information tracks prescription drugs from manufacturers, to warehouses, and to pharmacies. Pharmacists or other drug dispensers and patients can check the CEDMN database to trace drugs to their sources. Drugs are also tracked and logged in the CEDMN using barcode scanning, RFID identifiers, and Electronic Data Interchange. In Japan, the National Database of Health Insurance Claims and Specific Health Checkups of Japan (NDB) provides information regarding prescription drugs and health insurance claims to the public. The Japanese Ministry of Health, Labour, and Welfare also disseminates information and statistics regarding prescription drugs.

For ease of description, the terms “prescription drug management program/programs” and “PDMP/PDMPs” as used herein will refer to any and all of the above programs and any other similar programs that exist now or in the future directed to the monitoring, managing, etc. of prescription drugs in any region, nation, or other jurisdiction.

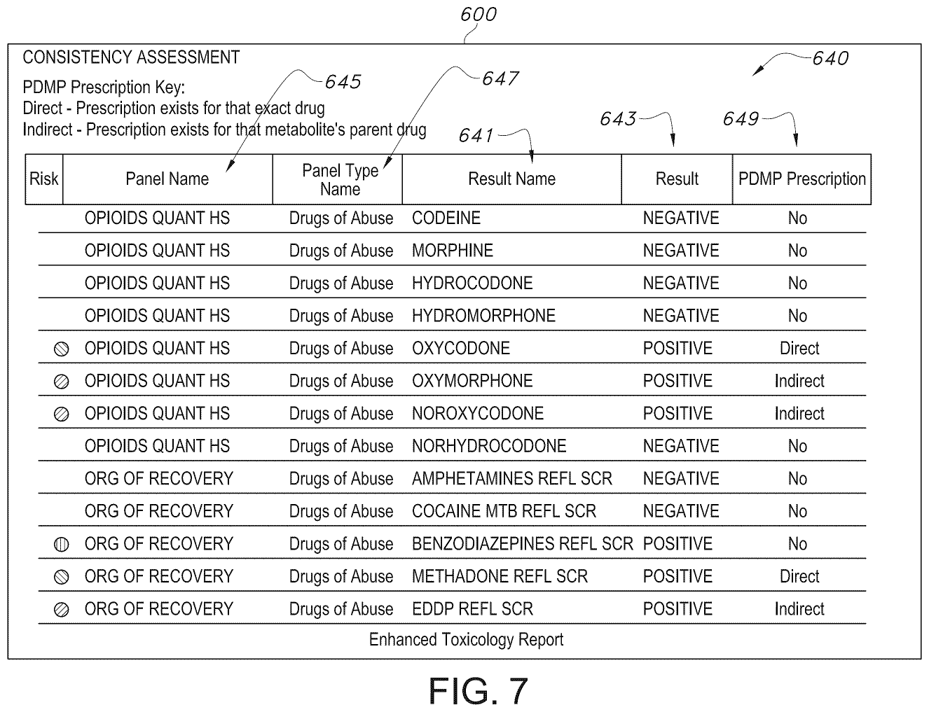

With further reference to FIG. 7, which illustrates a second view of the example enhanced toxicology report 600, in certain aspects the enhanced toxicology report 600 can include a toxicology screen report breakdown 640. The toxicology screen report breakdown 640 can include a detailed list 641 of the various drugs/controlled substances that were tested by the laboratory 510, as well as the results 643 of those tests. The toxicology screen report breakdown 640 can further include a panel name of the test 645, a type of the panel tested 647, and a PDMP prescription section 649. The PDMP prescription section 649 can be indicative of the correlation between the tested drugs/controlled substances and the controlled substance prescription data from the PDMP 530.

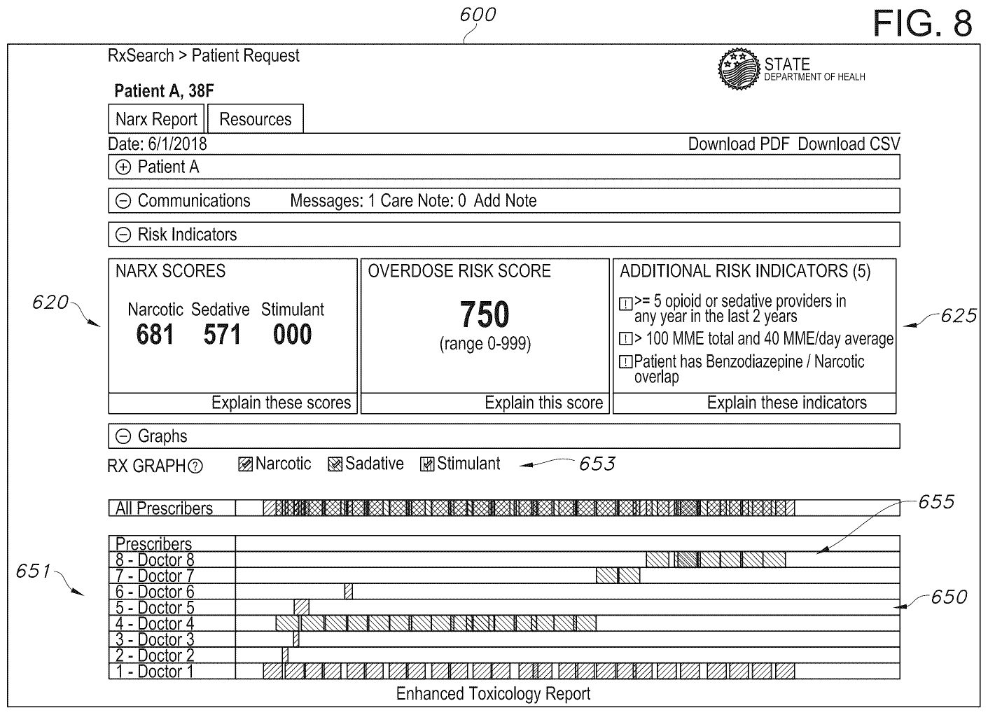

In yet another view (illustrated in FIG. 8), the enhanced toxicology report 600 can include a graphical element 650 that is indicative of prescriptions of controlled substances issued to the patient for a relevant time period. The graphical element 650 as shown includes a list 651 of prescribers that issued the prescriptions, a legend 653 for identifying the drug identity for each prescription, as well as a graph 655 that illustrates the overlap of prescriptions of controlled substances issued to the patient for the relevant time period from multiple prescribers. The enhanced toxicology report 600 shown in FIG. 8 also includes a different representation of the overdose risk score 620 in which one or more additional risk indicators 625 are included. These additional risk indicators 625 are risk categories to which the patient may match and can include, for example only, drug inconsistency, doctor shopping, and/or dangerous drug combinations.

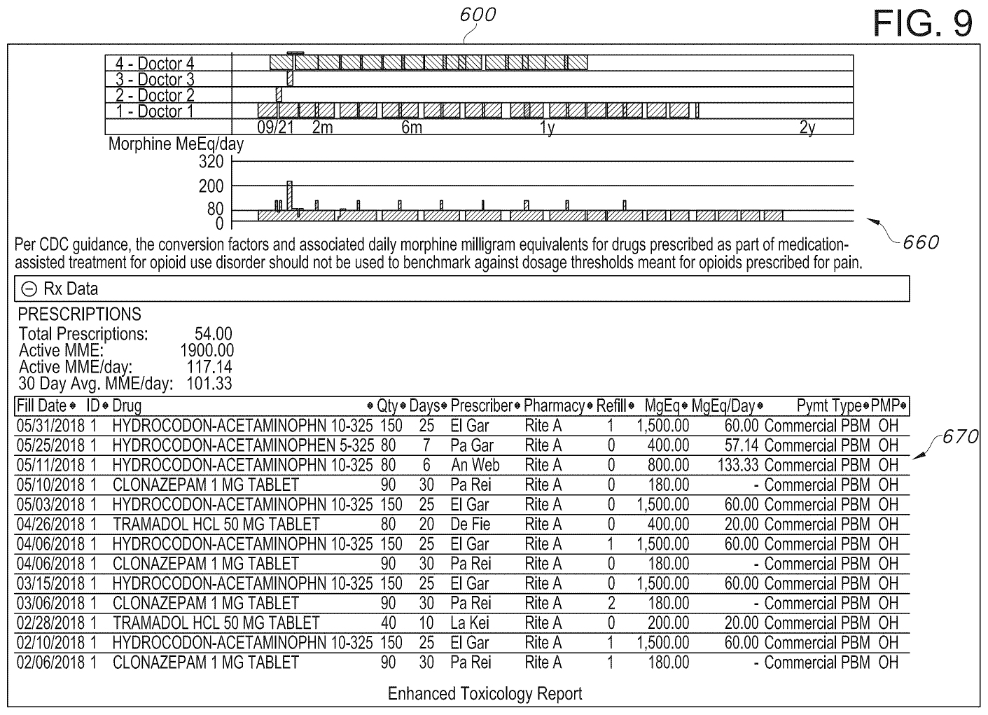

With further reference to FIG. 9, the enhanced toxicology report 600 can include an indication of the determined daily morphine milligram equivalent for the patient for a given time period. For example only, the enhanced toxicology report 600 can include a historical trend 660 of the determined daily morphine milligram equivalent for the patient for a given time period (such as over the previous two years). In some aspects, this historical trend 660 of the determined daily morphine milligram equivalent for the patient can be presented in a graphical format as shown in FIG. 9, in which the historical trend 660 is shown in a bar graph. Other formats for indicating the determined daily morphine milligram equivalent for the patient for a given time period are contemplated by the present disclosure. In certain implementations, the enhanced toxicology report 600 can also include a detailed prescription history 670 of the patient. The detailed prescription history 670 can include various details of the prescriptions issued to the patient, including but not limited to the date of prescription, the drug type/name, the quantity, the number of days of prescription provided, the prescriber, the filling pharmacy, an indication of the number of refills, and the strength.

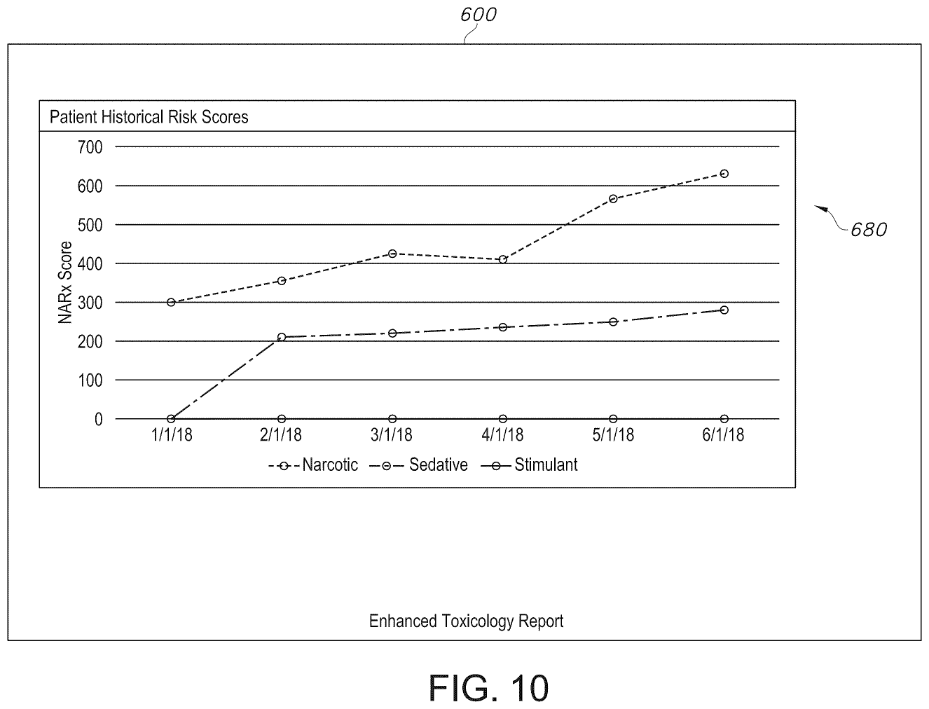

In certain implementations, the enhanced toxicology report 600 can also or alternatively include a historical trend 680 of the determined overdose risk scores 620 of the patient as shown in FIG. 10. The historical trend 680 of the overdose risk score 620 can be a line graph (as shown in FIG. 10) or any other suitable format that provides a simple, visual indication of the changes in the determined overdose risk scores 620 of the patient. It should be appreciated that the enhanced toxicology report 600 can include any one or any combination of the features illustrated in FIG. 6-FIG. 10. Further, in some aspects a recipient of the enhanced toxicology report 600 can originally be presented with a brief summary of the information contained within the enhanced toxicology report 600. Through interaction with links, tabs, or other user interface elements similar to a webpage, the recipient may switch between various views and information in the enhanced toxicology report 600. The ability to switch between various views and presentations of the information can be beneficial to the various recipients of the enhanced toxicology report 600, e.g., in order to quickly and simply find the information most relevant to a particular recipient.

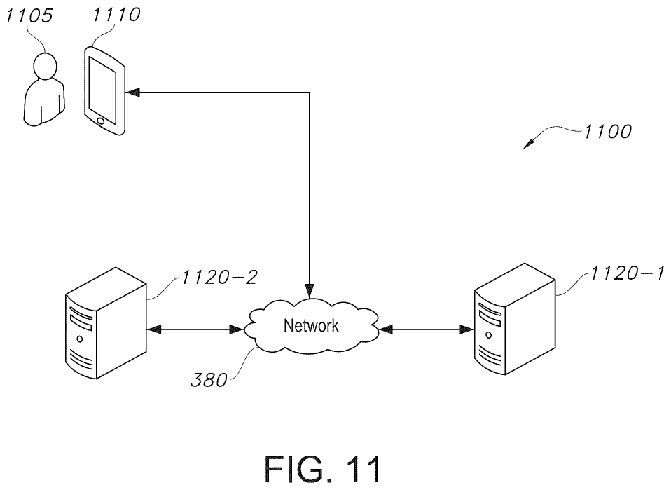

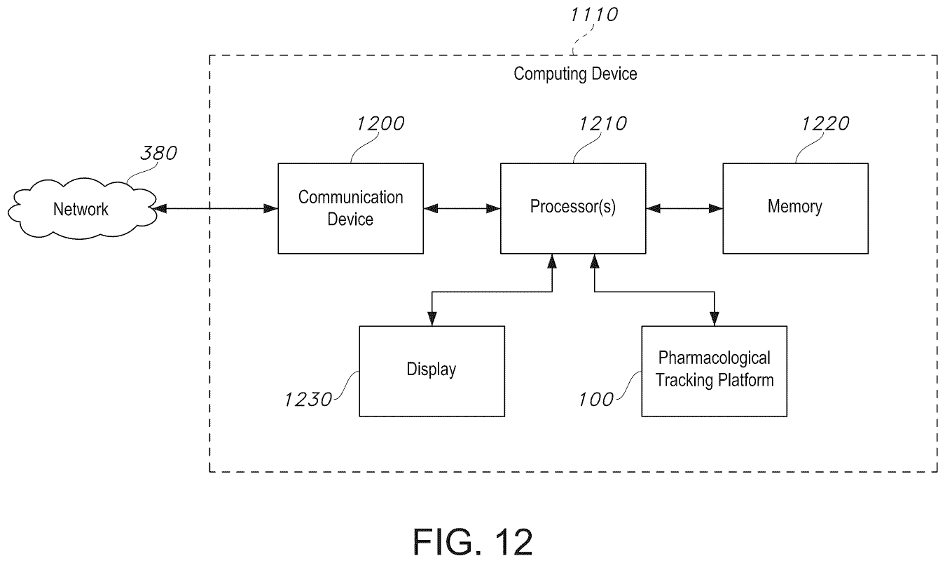

Referring now to FIG. 11, a diagram of an example computing system 1100 is illustrated. The computing system 1100 can be configured to implement the pharmacological tracking platform 100 described herein, e.g., amongst a plurality of users 1105 via their computing devices. The computing system 1100 can include one or more example computing devices 1110 and one or more example servers 1120 that communicate via a network 380 (as described above) according to some implementations of the present disclosure. For ease of description, in this application and as shown in FIG. 11, one example computing device 1110 and two example server computing devices 1120 (server computing devices 1120-1 and 1120-2) are illustrated and described. It should be appreciated, however, that there can be more computing devices 1110 and more or less server computing devices 1120 than is illustrated. While illustrated as a mobile phone (a “smart” phone), each computing device 1110 can be any type of suitable computing device, such as a desktop computer, a tablet computer, a laptop computer, a wearable computing device such as eyewear, a watch or other piece of jewelry, or clothing that incorporates a computing device. A functional block diagram of an example computing device 1110 is illustrated in FIG. 12.

The computing device 1110 is shown as including a communication device 1200, one or more processors 1210, a memory 1220, a display device 1230, and the pharmacological tracking platform 100. The processor(s) 1210 can control the operation of the computing device 1110, including implementing at least a portion of the techniques of the present disclosure. The term “processor” as used herein is intended to refer to both a single processor and multiple processors operating together, e.g., in a parallel or distributed architecture.

The communication device 1200 can be configured for communication with other devices (e.g., the server computing devices 1120 or other computing devices 1110) via the network 380. One non-limiting example of the communication device 1200 is a transceiver, although other forms of hardware are within the scope of the present disclosure. The memory 1220 can be any suitable storage medium (flash, hard disk, etc.) configured to store information. For example, the memory 1220 may store a set of instructions that are executable by the processor 1210, which cause the computing device 1110 to perform operations (e.g., such as the operations of the present disclosure). The display device 1130 can display information to the user 1105. In some implementations, the display device 1230 can comprise a touch-sensitive display device (such as a capacitive touchscreen and the like), although non-touch display devices are within the scope of the present disclosure.

It should be appreciated that the example server computing devices 1120 can include the same or similar components as the computing device 1110, and thus can be configured to perform some or all of the techniques of the present disclosure. Further, while the techniques of the present disclosure are described herein in the context of the pharmacological tracking platform 100, which is illustrated as being a component of the computing device 1110, it is specifically contemplated that each feature of the techniques may be performed by a single computing device 1110 alone, a plurality of computing devices 1110 operating together, a server computing device 1120 alone, a plurality of server computing devices 1120 operating together, and a combination of one or more computing devices 110 and one or more server computing devices 1120 operating together.

In embodiments, the platform 100 may be configured to predict diabetes. The platform 100 may also be configured to predict prediabetes. By way of these examples, the platform may predict in a patient prior to diagnosis of diabetes or prediabetes by a medical professional. In these embodiments, the platform may predict diabetes or prediabetes based holistic medical data, based on data from human interaction techniques, and one or more combinations thereof. The holistic medical data and the human interaction techniques may include one or more of customer data matching, panel matching, incentivization or activities, and overlays of health care data. In some embodiments, the determination of diabetes or prediabetes can be based on the matching and similarities found in the holistic medical data and data from human interaction techniques that may be derived with and implemented by using deep learning techniques.

In embodiments, the platform 100 may be configured to calculate a clinical diabetes risk score. The platform 100 may also be configured to calculate a clinical prediabetes risk score. The platform may be configured to identify characteristics indicative of prediabetes or diabetes such as by using one or more of clinical variables, biological variables, and polymorphisms. By way of these examples, the platform can be configured to provide a clinical diabetes risk score based on the identification of characteristics that predict later diabetes using variables available in the clinical setting as well as biological variables and polymorphisms. In embodiments, the platform has been shown to facilitate conclusions that can be shown to support that one very effective clinical predictor of diabetes is adiposity and baseline glucose can be a very effective biological predictor. In many examples, observations of adiposity and baseline glucose may be shown to outweigh conclusions based on clinical and biological predictors subject to gender differences or affected by genetic polymorphisms.

In many instances, there are many examples of detailing the risk for diabetes or prediabetes such as those based on the anthropometric variables associated with diabetic levels of fasting glucose and found that BMI, waist circumference, and waist-to-hip ratio, and other suitable holistic medical data and data from various human interaction techniques.

In embodiments, the platform 100 may be configured to calculate patient risk regarding a plurality of medical conditions, such as diabetes, heart disease, cancer, and osteoporosis. The platform may be configured to calculate the patient risk via one or more of panel data matching, multistage analytics, semantic model building, axiomatic model building, and data analysis, monitoring, and filtering, such as over a period of time. In some embodiments, the platform may use data derived from a health risk assessment (HRA) to calculate the patient risk. The HRA may include one or more of a questionnaire, a risk score, and a report including feedback, the feedback including potential areas of improvement. The questionnaire may include one or more questions directed to the patient regarding nutrition, fitness, biometrics such as blood pressure and/or cholesterol, stress, sleep, and mental health. The report including feedback may include feedback related risk of chronic conditions such as heart disease, diabetes, cancer, and obesity.

In embodiments, the platform may be configured to perform a multi-stage analysis to handle and parse patient data. The multi-stage analysis may include analysis using an empirical model such as a machine learning model, a deep learning model, or a combination thereof. The multi-stage analysis may include analysis using a semantic model, such as a model that allows for deep semantic information to be constructed relating to data. The multi-stage analysis may include rules relating to an application of the semantic model. The multi-stage analysis may include rules implemented by one or more medical technologists. In some embodiments, the semantic model may be or include one or more abstracted semantics-based descriptions of data, the capturing and/or representing a meaning of data and/or how data is shared and/or propagated through the platform.

In embodiments, the various systems and methods of the present disclosure include a medical records system configured for analyzing workflow for clinical professionals. The system can include a healthcare database configured to store a plurality of medical records. The medical records include: demographic records including one or more of height, weight, sex, gender, ethnicity, and age of a plurality of patients; diagnosis records including medical diagnoses of the patients; prescription records including drugs previously prescribed to the patient; and testing records including tests ordered for the patients, tests performed on the patients, and results of tests performed on the patients, the testing history including names and codes used to identify the tests by health care centers and labs ordering and/or administering the tests. The system can include a machine learning device in communication with the healthcare database and configured to receive the demographic records, the diagnosis records, the prescription records, and the testing records from the healthcare database. The machine learning device is configured to train an artificial intelligence based on the demographic records, the diagnosis records, the prescription records, and the testing records. The artificial intelligence is trained to identify inconsistencies in the names and/or codes used by the health care centers and labs and normalize the names and codes used to identify the tests by health care centers such that similar tests are identified by consistent names and/or codes within the healthcare database. The artificial intelligence is trained to analyze the tests being ordered by individual health care centers of the health care centers to determine redundancies in testing by the individual health care centers. The artificial intelligence is also trained to analyze the demographic records, diagnosis records, prescription records, and testing records to identify inconsistencies in diagnosing, drug prescribing, and test ordering practices of the health care facilities, identify potential improvements to the diagnosing, drug prescribing, and test ordering practices of the health care facilities, and identify medically unnecessary and/or redundant diagnosing, drug prescribing, and test ordering practices of the health care facilities.

In embodiments, the platform 100 may store data such as patient data and/or clinical data in one or more arrays and process data in a flexible manner such that data is readily accessible to end users for querying. The platform may be configured such that an end user may query the data using one or more taxonomy-based query tools, such as SQL or SparQL, thereby facilitating data query by graphic user interfaces and/or natural language-based query interfaces.

In embodiments, the platform 100 may include a convolutional neural network configured to identify patterns in data and use the patterns to make determinations, such as determinations related to a patient, a healthcare provider, and/or a treatment plan or regimen. The determinations may relate to evaluation of efficacy of a healthcare provider and/or a treatment plan or regimen, and/or to prediction or and/or recommendation of a healthcare provider and/or treatment plan or regimen. The convolutional neural network may combine patient data such as genetics and prescription data to identify patterns. In some embodiments, the convolutional neural network may also use data related to patient behavior to identify patterns. In some embodiments, the convolutional neural network may use one or more underlying factors such as economic wealth, education and/or general health and lifestyle of a patient to recognize patterns and/or make determinations. In some embodiments, the platform may be configured to use the convolutional neural network and patterns derived therefrom to determine optimal outcomes for a patient and/or determine paths to the determined optimal outcome, such as patient recovery, disease remission, and/or elimination or substantial decrease of patient risk factors.

In embodiments, the platform includes and/or implements a feedback loop to analyze and/or organize health care and patient data from one or more healthcare providers and patients. The feedback loop may intake data related to treatment acceptance, social media response, one or more responses and/or reactions to a medication and/or a treatment program, or any other suitable type of information.

In embodiments, the various systems and methods of the present disclosure include a medical records system configured for identifying abuse and/or misuse practices of a medical patient and a healthcare database in communication with a state-based prescription drug monitoring program database and configured to store a plurality of medical records. The medical records include demographic records including one or more of height, weight, sex, gender, ethnicity, and age of the patient; diagnosis records including medical diagnoses of the patient; and prescription records including drugs previously prescribed to the patient, drugs being taken by the patient as reported by the patient, and drug prescription records of the patient received from the prescription drug monitoring program database. The medical records system is configured to identify abuse and/or misuse of prescription drugs by the patient based on the drugs previously prescribed, the drugs being taken, and the drug prescription records received from the prescription drug monitoring program database.

In embodiments, the platform may include a data exchange revenue management module configured to provide optionality to a patient regarding treatment options versus price.

In embodiments, the platform is configured to store a demographic dataset that facilitates matching of clinical trials to suitable patients therefor based on demographics of the patients. The demographics may be related to patients within regional boundaries and/or may include one or more of optimization factors, economic wealth, population demographics, and outcome prediction.

In embodiments, the platform may include a referral engine configured to facilitate, track, and/or analyze referrals of patients to and from health care providers. The referral engine may implement one or more balancing factors to account for economic considerations related to patient referrals.

In embodiments, the platform may include a master record configured to serve as a reliable reference related to one or more of patients, health care providers, treatment plans, and any other suitable data. The master record may include handled data such as analyzed, filtered, and/or deduplicated data. In some embodiments, analyzing, filtering, and/or deduplicating via the platform for the master record may include use of machine learning, fuzzy logic, neural networks, or any other suitable process for data handling. The platform may aggregate and analyze, filter, and/or deduplicate data from a plurality of sources, such as health care databases, electronic medical records, state prescription drug monitoring programs (PDMPs), or any other suitable source of data. For example, the platform may be configured to process patient data related to a particular patient name, the patient name being related to one or more other categories of patient information, such as physiological data, treatment history, and prescription history, and the patient name and patient information being aggregated from different and inconsistent sources. The platform may analyze the patient name and the related patient information via a neural network and make a determination to consolidate, deduplicate, correct, and/or normalize the patient name and patient information such that the patient name and the patient information are reliably correlated with one another and stored in the master record. In some embodiments, patient information stored in the master record and handled data related thereto may include genetic data. The platform may be configured to use genetic data to predict and/or determine family and/or lineage of one or patients and may use determined family and/or lineage information for further predictions and analyses. In some embodiments, the platform may be configured to determine one or more ethnic origins of a patient name or collection of related patient names and use determined ethnic origins to identify and correct for misspellings and/or other inconsistencies between possibly related patient data.

In some embodiments, the platform may include a panel matching engine configured to identify, account for, and/or correct inconsistencies in treatment and/or test panel records. Healthcare providers may use inconsistent names and/or identifiers for one or more treatment panels and/or test panels that are substantially equivalent. The panel matching engine is configured to analyze treatment and/or test panel data to consolidate, deduplicate, and/or normalize test panels. In some embodiments, the panel matching engine may implement one or more of machine learning, fuzzy logic, and/or neural networks to handle treatment and/or test panel data. For example, where a particular healthcare system uses inconsistent identifiers for a standard test panel, or where two different healthcare systems use inconsistent identifiers for the standard test panel, the panel matching engine may intake the inconsistent identifiers, determine that each of the inconsistent identifiers is used to identify the standard test panel, and correlate each of the inconsistent identifiers to the standard test panel within databases of the platform such that the platform and modules and engines thereof may consistently process and analyze the test panel and information related thereto.

In embodiments, the platform may be configured to integrate patient data related to a lifestyle of a patient with other patient data. The platform may predict lifestyle data and integrated predicted lifestyle data with other patient data. Other patient data integrated with lifestyle data may include demographic data, prescription history, treatment history, diagnosis history, genetic information, name, age, social security number, and/or any other suitable patient data.

In embodiments, the platform may be configured to recommend test panels for one or more patients. Recommendations of test panels may be derived from patient data, previous panel results, costs of test panels to patients and/or health care providers, or any other suitable information. In some embodiments, the platform may implement machine learning, fuzzy logic, and/or a neural network in test pane recommendation.

In embodiments, the various systems and methods of the present disclosure include a medical records system configured for integrating traditional medical records with lifestyle, wellness, and physiological monitoring information and disseminating the same. The system includes a healthcare database configured to store a plurality of medical records. The medical records include demographic records including one or more of height, weight, sex, gender, ethnicity, and age of a plurality of patients; diagnosis records including medical diagnoses of the patients; prescription records including drugs previously prescribed to the patient; testing records including tests ordered for the patients, tests performed on the patients, and results of tests performed on the patients, the testing history including names and codes used to identify the tests by health care centers and labs ordering and/or administering the tests. The healthcare database is further configured to store lifestyle and wellness records of the patients, the lifestyle and wellness information including information related to one or more of diet, smoking, drinking, and exercise habits. The healthcare database is configured to transmit the medical records and the lifestyle and wellness records to health care facilities and to third parties.

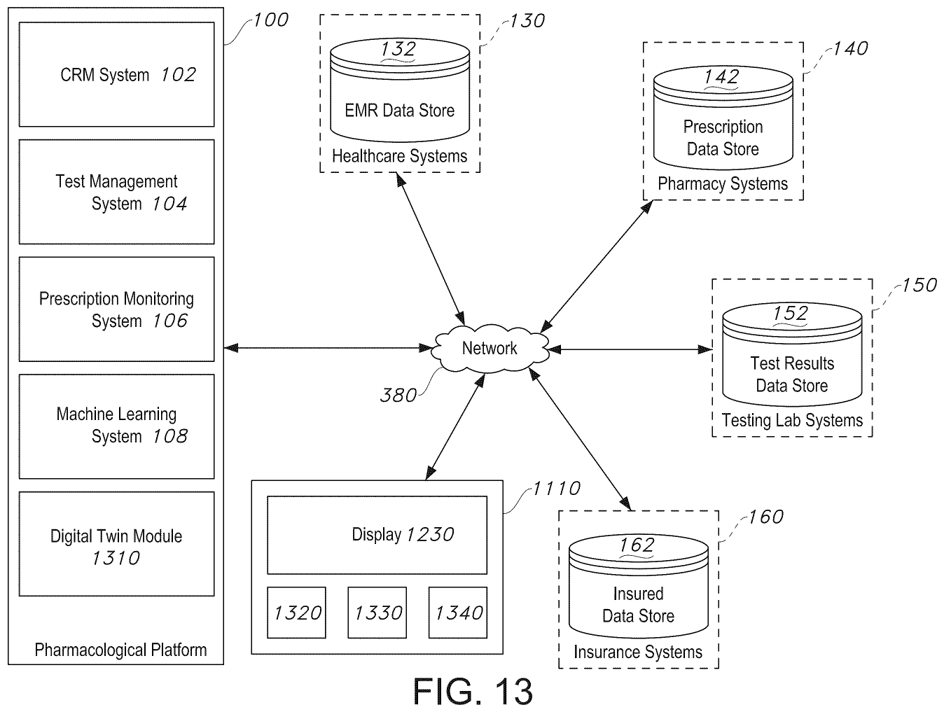

In some embodiments, the platform 100 may form at least one digital twin based on health information containing data related to a patient. In some embodiments, the pharmacological platform 100 may include a digital twin functionality, at 1300 in FIG. 13, which may be facilitated by and/or displayed at one more computing devices 1100 connected through the network to the platform 100. The one or more digital twins may be visualized at one or more computing devices or by one or more computing devices and displayed or accessible from one or more locations on the network. The platform 100 may facilitate one or more digital twins while maintaining necessary restrictions on access to the data that can be at least one of visualized, simulated, compared to other digital twins, and the like. In embodiments, the health information related to the patient may include one or more of medical records such as those stored in an EMR, prescription data indicative of past and/or present prescriptions the patient may have taken or be taking. In embodiments, the health information related to the patient may include test data indicative of past and/or present medical tests performed on the patient and results thereof. In embodiments, the health information related to the patient may include insurance information indicative of past or present insurance plans and claims and information related thereto. In embodiments, the health information related to the patient may include physiological information such as age, height, weight, blood pressure, etc. of the patient, genetic information such as DNA test results and/or information related to ancestry and/or lineage of a patient and information related to health and/or genetics of relatives of the patient. In embodiments, the health information related to the patient may include data regarding one or more disease conditions of the patient. In embodiments, the health information related to the patient may include healthcare provider information such as information indicative of past and/or present doctors, nurses, surgeons, physician's assistants, and other healthcare professionals who have worked on treating, testing, performing surgery on, or otherwise caring for the patient, the population of patients, and/or other patients in a healthcare environment. The platform 100 may provide multiple machine learning modules from which the data from the various providers can be understood and patterns or deviations therefrom can be determined about patients, providers, and interaction in the medical delivery. The multiple machine learning modules can be deployed from or by the machine learning system 108. The multiple machine learning modules may be connected to or associated with the machine learning system 108 and may be available over the network 308. In some embodiments, the health information received by the platform 100 may include information related to a lifestyle and/or an economic status or socioeconomic position of the patient. In some embodiments, the health information received by the platform 100 may include interactions with one or more healthcare providers and compliance with recommended treatment plans by the one or more healthcare providers. In some embodiments, the health information may further include health information derived from the patient during medical research in which the patient took place, such as one or more clinical trials. In some embodiments, the health information may include personally entered healthcare data derived directly from the patient and/or the population of patients. In some embodiments, the health information may include an entire medical record of the patient and/or the patients included in the population of patients. In some embodiments, the platform 100 may receive the health information from one or more of an EMR, a prescription database such as the PDMP system 530, an insurance database, a healthcare research database, or any other suitable source of health information. In some embodiments, the platform 100 may receive the health information via the network 380 and/or via the communication system 240. The platform 100 may store the health information in one or more of the EMR data store 132, the prescription data store 142, the test results data store 152, the insured data store 162, the patient data store 230, the physician data store 226, the clinic data store 222, the PDMP system 530, or any other suitable digital storage medium.

In some embodiments, the platform 100 may include a digital twin module 1302 in communication with one or more data stores and/or processing units of the platform 100 and configured to receive the health information and create the digital twin of the patient based on the received health information at or using the computing devices 1110 (FIG. 12). The digital twin of the patient may be a digital representation of at least one health state of the patient. In many examples, the one or more digital twins can be displayed on the computing device in one or more instances such as digital twin 1320, 1330, 1340, and the like. For example, in some embodiments, the digital twin of the patient may be a digital representation of an entire body of the patient, of a biological system of the patient such as the cardiovascular system or the respiratory system, and/or of an organ of the patient such as a lung, a liver, or a heart of the patient. In some embodiments, the digital twin of the patient may be an abstract digital representation of the at least one health state of the patient, such as a digital representation of risk factors contributing to diabetes, prediabetes, heart disease, or any other suitable disease, syndrome, disorder, or health state of the patient. In some embodiments, the digital twin of the patient may include one or more of numbers, trends, predictions, charts, graphs, thresholds, ranges, 2-dimensional models, and/or 3-dimensional models of the patient, the one or more health states of the patient, risk factors of the patient, biometrics of the patient, data derived from health information related to the patient, and/or any other suitable metrics and/or information related to the patient. In some embodiments, the platform 100 may be configured such that the digital twin of the patient may include health information related to the patient and may be compared to ideal disease state data, the ideal disease state data being based upon one or more clinical standards and/or optimal health outcomes.

In some embodiments, the platform 100 may form at least one digital twin based on health information containing data related to a population of patients. The health information related to the population of patients may include one or more of medical records such as those stored in an EMR, prescription data indicative of past and/or present prescriptions the population of patients may have taken or be taking, test data indicative of past and/or present medical tests performed on the population of patients and results thereof, insurance information indicative of past or present insurance plans and claims and information related thereto, physiological information such as age, height, weight, blood pressure, etc. of the population of patients, genetic information such as DNA test results and/or information related to ancestry and/or lineage of a population of patients and information related to health and/or genetics of relatives of the population of patients, healthcare provider information such as information indicative of past and/or present doctors, nurses, surgeons, physician's assistants, and other healthcare professionals who have worked on treating, testing, performing surgery on, or otherwise caring for the population of patients in a healthcare environment. In some embodiments, the health information may further include health information derived from the population of patients during medical research in which the population of patients took place, such as one or more clinical trials.

Patients included in the population of patients may be related to one another, such as by similarities in one or more of medical records such as those stored in an EMR, prescription data indicative of past and/or present prescriptions the population of patients may have taken or be taking, test data indicative of past and/or present medical tests performed on the population of patients and results thereof. In embodiments, patients included in the population of patients may be related to one another, such as by similarities in insurance information indicative of past or present insurance plans and claims and information related thereto. In embodiments, patients included in the population of patients may be related to one another, such as by similarities in physiological information such as age, height, weight, blood pressure, etc. of the population of patients. In embodiments, patients included in the population of patients may be related to one another, such as by similarities in genetic information such as DNA test results and/or information related to ancestry and/or lineage of a population of patients and information related to health and/or genetics of relatives of the population of patients, healthcare provider information such as information indicative of past and/or present doctors, nurses, surgeons, physician's assistants, and other healthcare professionals who have worked on treating, testing, performing surgery on, or otherwise caring for the population of patients in a healthcare environment. In some embodiments, the patients included in the population of patients may be related to one another according to health information derived from the population of patients during medical research in which the population of patients took place, such as one or more clinical trials. In some embodiments, the platform 100 may use the digital twin module 1302, digital twins of a plurality of patients, digital twins of one or more populations of patients, in combination with one or more of the machine learning modules to facilitate the determination of similarities in the health information between one or more patients and/or to form a population of patients based on the health information, such as by determining based on the health information and/or the digital twins that one or more patients have similarities in lifestyle, diet, exercise, health state, diagnosis, prognosis, present and/or past treatments and/or treatment plans, or any other suitable health property for grouping a plurality of patients.

In some embodiments, the platform 100 may be configured to determine one or more health care professionals who are suited to treat the patient and/or the population of patients having one or more similarities. The platform 100 may be configured to determine the one or more health care professionals based on the health information wherein the health information includes data related to experience and/or expertise of the one or more health care professionals. The platform 100 may compare the data related to experience and/or expertise of the one or more health care professionals to the health information of the patient and/or the population of patients and determine that the one or more health care professionals are particularly suited to treat the patient and/or the population of patients based on the comparison. The platform 100 may then display the determination that the one or more health care professionals are suited to treat the patient and/or the population of patients to the user of the platform 100. For example, the platform 100 may receive health information indicating that one or more clinicians are particularly suited to treat and/or are experts in treating a disease such as diabetes, and may determine or have determined that a population of patients is at risk of diabetes and/or has developed diabetes. The platform 100 may output to the user of the platform 100 a recommendation that the one or more clinicians that are particularly suited to treat and/or are experts in treating diabetes be associated with the patient and/or the population of patients who are at risk of diabetes and/or have developed diabetes such that the patient and/or the population of patients can be treated by the one or more clinicians.

In some embodiments, the digital twin module 1302 may be configured to receive the health information and create the digital twin of the population of patients based on the received health information, which can be displayed on one or more computing devices. The digital twin of the population of patients may be a digital representation of at least one health state of the population of patients. For example, in some embodiments, the digital twin of the population of patients may be a digital representation of an entire body of the population of patients, of a biological system of the population of patients such as the cardiovascular system or the respiratory system, and/or of an organ of the population of patients such as a lung, a liver, or a heart of the population of patients. The digital twin of the patients may be derived from averaging data and/or trends in the health information of the population of patients. For example, where the digital twin is a digital representation of an entire body, a biological system of the population of patients, or of an organ of the population of patients, the digital twin module 1302 may process the health information to determine an average, typical, or otherwise appropriate digital representation of the population of patients. For example, where the population of patients may be similar in that the patients included in the population of patients are at risk of liver failure, the digital twin module 1302 may process the health information to create a digital representation of a liver typical and/or related physiological objects and/or metrics relevant to the risk of liver failure in the population of patients. In some embodiments, the digital twin of the population of patients may be an abstract digital representation of the at least one health state of the population of patients, such as a digital representation of risk factors contributing to diabetes, prediabetes, heart disease, or any other suitable disease, syndrome, disorder, or health state of the population of patients. In some embodiments, the one or more digital twins of the population of patients may include one or more of numbers, trends, predictions, charts, graphs, thresholds, ranges, 2-dimensional models, and/or 3-dimensional models of the population of patients, the one or more health states of the population of patients, risk factors of the population of patients, biometrics of the population of patients, data derived from health information related to the population of patients, and/or any other suitable metrics and/or information related to the population of patients. In embodiments, the one or more digital twins may be displayed by or the visualization may be facilitated by the one or more computing devices, which may access or utilize additional resources available from the network. In some embodiments, the platform 100 may be configured to determine one or more patients included in the population of patients that are more or less likely to develop one or more diseases. For example, the platform 100 may determine that certain members of the population of patients are more likely than other members of the population of patients to develop common diseases, such as breast cancer or heart disease, or less common diseases such as cystic fibrosis.

In some embodiments, the platform 100 may be configured to present the digital twin of the patient and/or the digital twin of the population of patients to a user of the platform 100. The digital twin may be presentable via one or more of text, numbers, trends, predictions, charts, graphs, thresholds, ranges, 2-dimensional models, and/or 3-dimensional models. The platform may be configured to present one or more of the digital twins of the patient and/or the digital twins of the population of patients via one or more of a monitor such as a computer monitor, a television, a projector, or a holographic display, a wearable device such as smart glasses, a VR headset, AR glasses, or any other suitable device or set of devices for presenting the digital twin of the patient and/or the digital twin of the population of patients. The platform may be configured to use one or computing devices to facilitate the presentation of the one or more digital twins in that the computing device can be integral to or associated with a monitor such as a computer monitor, a television, a projector, or a holographic display, a wearable device such as smart glasses, a VR headset, AR glasses, or any other suitable device or set of devices for presenting the digital twin of the patient and/or the digital twin of the population of patients in association with the computing device. In some embodiments, such as those wherein the digital twin of the patient and/or the digital twin of the population of patients includes one or both of 2D and 3D models, the platform 100 may be configured to present the digital twin of the patient and/or the digital twin of the population of patients such that the digital twin is manipulatable by the user of the platform 100 in real time by the user of the platform 100. In some embodiments, the platform 100 may be configured such that the digital twin of the patient may include health information related to the patient and may be compared to ideal disease state data, the ideal disease state data being based upon one or more clinical standards and/or optimal health outcomes.

In some embodiments, the one or more machine learning modules of the platform 100 may be in communication with the digital twin module 1302 and may be configured to perform machine learning techniques to process, enhance, augment, transform, analyze, and/or make simulations and/or predictions related to one or more of the health information, the one or more digital twins of the patient, and the one or more digital twins of the population of patients. The one or more machine learning modules may be configured to perform machine learning tasks on the health information to process, enhance, augment, transform, analyze, and/or make simulations and/or predictions related to the health information, for example to group the health information, relate pieces of the health information to one another, relate pieces of the health information to the patient, to one or more patients included in the population of patients, to related pieces of the health information to the population of patients, to relate one or more patients included in the population of patients to one another, to determine which patients to include in the population of patients by drawing one or more similarities between one or more patients, or any other suitable use of the health information. In some embodiments, the one or more machine learning modules may be configured to train using the health information and use machine learning techniques to analyze the health information and make predictions based thereon related to the patient and/or the population of patients. For example, the one or more machine learning modules may be configured to use machine learning techniques to determine whether the patient is at risk for a disease based on training of the one or more machine learning modules based on the health information. Such training of the one or more machine learning modules may allow the one or more machine learning modules to make determinations and/or predictions regarding one or more health states of the patient and/or the population of patients that are not otherwise achievable by research, diagnosis, and analysis of patients and/or populations of patients. For example, the one or more machine learning modules may use machine learning and/or deep learning techniques and training on the health information to determine that a patient is at high risk to develop a disease such as heart disease, diabetes, liver failure, or any other suitable health issue by finding correlations in the health information related to the patient and/or the population of patients where traditional diagnosis, testing, and/or research may not otherwise uncover the same correlations related to such diseases and/or health states.

In some embodiments, the digital twin module 1302 may be configured to simulate one or more potential future health states of the patient using one or more of the digital twins of the patient, the digital twin of the population of patients, and the one or more machine learning modules. The one or more machine learning modules may intake the digital twin of the patient and, using machine learning and/or deep learning and training related thereto, simulate a plurality of future health states of the patient. The future health states of the patient may be simulated according to variables, such as a time frame, a treatment schedule, a prescription drug schedule, a lifestyle, potential developments in one or more health issues experienced by the patients, any other suitable variable for use in simulation, and/or a combination thereof. Simulating based on time frame may include simulating a potential health state of the patient in one or more, seconds, minutes, hours, days, months, years, or any other suitable time frame. For example, the digital twin module 1302 may simulate a health state of an ER patient 90 seconds into the future relative to present time, or of a prediabetes patient three years into the future relative to present time. For example, the digital twin module 1302 may simulate a health state of a patient suffering from partial paralysis according to a first physical therapy treatment plan and a second physical therapy treatment plan. For example, the digital twin module 1302 may simulate a health state of a heart disease patient according to potentially prescribing heart disease medication to the patient and advising that the patient take a small dose of aspirin regularly. For example, the digital twin module 1302 may simulate a health state of the patient according to a regimented exercise and dict plan and the patient continuing a current exercise and diet plan thereof. For example, the digital twin module 1302 may simulate a future health state of a patient having a tumor according to whether the tumor becomes cancerous and whether the tumor remains benign. The previous examples are intended to be non-limiting and illustrate a portion of a potential scope of simulations that the one or more digital twin module 1302 modules may perform. In some embodiments, the digital twin module 1302 may simulate a future health state of the patient based on a plurality of variables, such as by simulating a health state of a patient according to a combination of time frames, treatment plans, prescription drug schedules, and lifestyle changes. In some embodiments, the digital twin module 1302 may be configured to update the digital twin of the patient according to one or more digital twins of the patient simulating one or more potential future health states based on one or more variables.

In some embodiments, the digital twin module 1302 may be configured to simulate one or more potential future health states of the population of patients using one or both of the digital twin of the population of patients, and the one or more machine learning modules. The one or more machine learning modules may intake the digital twin of the population of patients and, using machine learning and/or deep learning and training related thereto, simulate a plurality of future health states of the population of patients. The future health states of the population of patients may be simulated according to variables, such as a time frame, a treatment schedule, a prescription drug schedule, a lifestyle, potential developments in one or more health issues experienced by the population of patients, any other suitable variable for use in simulation, and/or a combination thereof. Simulating based on time frame may include simulating a potential health state of the population of patients in one or more, seconds, minutes, hours, days, months, years, or any other suitable time frame. For example, the digital twin module 1302 may simulate a health state of a population of ER patients 90 seconds into the future relative to present time, or of a population of prediabetes population of patients three years into the future relative to present time. For example, the digital twin module 1302 may simulate a health state of a population of patients suffering from partial paralysis according to a first physical therapy treatment plan and a second physical therapy treatment plan. For example, the digital twin module 1302 may simulate a health state of a population of heart disease patients according to potentially prescribing heart disease medication to the population of patients and advising that the population of patients take a small dose of aspirin regularly. For example, the digital twin module 1302 may simulate a health state of the population of patients according to a regimented exercise and diet plan and the population of patients continuing a current exercise and diet plan thereof. For example, the digital twin module 1302 may simulate a future health state of a population of patients having a tumor according to whether the tumor becomes cancerous and whether the tumor remains benign. The previous examples are intended to be non-limiting and illustrate merely a portion of a potential scope of simulations that the one or more digital twin module 1302 modules may perform. In some embodiments, the digital twin module 1302 may simulate a future health state of the population of patients based on a plurality of variables, such as by simulating a health state of a population of patients according to a combination of time frames, treatment plans, prescription drug schedules, and lifestyle changes. In some embodiments, the digital twin module 1302 may be configured to update the digital twin of the population of patients according to one or more digital twins of the population of patients simulating one or more potential future health states based on one or more variables.

In some embodiments, the platform 100 may be configured to simulate one or more future health states of the patient and/or the population of patients using the digital twin module 1302 based on variables determined by the one or more machine learning modules and/or the user of the platform 100. The one or more machine learning modules may be configured to, based on training on the health information, determine variables that may lead to simulations of the one or more future health states of the patient and/or the population of patients via the digital twin module 1302 that may be useful to a healthcare professional, such as the user of the platform 100 in diagnosing, analyzing, researching, or otherwise learning from the digital twin including the simulation of the one or more potential future health states of the patient and/or the population of patients. In some embodiments, the platform 100 may be configured to receive one or more of the variables from a healthcare professional, such as the user of the platform 100, and form one or more simulated digital twins of the patient and/or the population of patients based on the one or more variables input to the platform 100 by the healthcare professional, such as the user of the platform 100.

In some embodiments, the platform 100 may be configured to continuously receive the health information and update the digital twin of the patient and/or the digital twin of the population of patients based on updated health information received subsequent to formation of the digital twin of the patient and/or the population of patients using the continuously received health information. The platform 100 may receive initial health information related to the patient and/or the population of patients and, using the digital twin module 1302, create an initial digital twin of the patient and/or the population of patients according to the initial health information received. Subsequent to receiving the initial health information, the platform 100 may receive updated health information related to the patient and/or the population of patients. Upon receiving the updated health information, the platform 100 and the digital twin module 1302 may update the digital twin of the patient and/or the population of patients based on the updated health information. In some embodiments, the platform 100 may determine differences in the initial health information versus the updated health information. The differences in the initial health information versus the updated health information may include, for example, changes in lifestyle, diet, exercise regimens, treatment plans, diagnosis, prognosis, health state, prescription drugs being taken, or any other suitable change in the health information related to the patient and/or the population of patients.

In some embodiments, the platform 100 may be configured to classify the differences in initial health information versus the updated health information using the one or more machine learning modules according to one or more machine learning and/or deep learning techniques. The one or more machine learning modules may use the differences in the initial health information versus the updated health information as training data and thereby learn to analyze and/or classify the differences in one or more ways that may be useful in analyzing the health information and/or predicting future disease states based on the health information, the initial health information, the updated health information, and/or a combination thereof.

In some embodiments, the platform 100 may be configured to receive healthcare research information from one or more healthcare research information sources and correlate the received healthcare research information with the health information, such as personally entered healthcare data, to determine one or both of relative accuracy of the healthcare research information and one or more discrepancies between the healthcare research information and the health information. The healthcare research information may be any data related to healthcare research, such as types of research performed, results of research, numbers of patients and/or subjects used in research, whether research was performed in vivo, in vitro, and/or in silico, identities of one or more patients and/or subject used in the research, tests performed in the research, drugs and/or treatments given and/or performed in the research, and/or any other suitable data related to healthcare research. The healthcare research information sources may include one or more of healthcare research institutes, healthcare research labs, other healthcare research organizations, one or more hospitals, labs, offices, testing centers, healthcare researchers, or any other suitable source of healthcare research information. The platform 100 may correlate the healthcare research information with the health information, the digital twin of the patient, the digital twin of the population of patients, and/or one or more machine learned models and/or predictions formed using the one or more machine learning modules to determine accuracy of the healthcare research information and/or determine one or more discrepancies between the healthcare research information and the health information, the digital twin of the patient, the digital twin of the population of patients, and/or the one or more machine learned models and/or predictions

In some embodiments, the platform 100 may be configured to perform impact discovery analysis using the one or more machine learning modules. The one or more machine learning modules may be configured to determine correlations between two or more healthcare research information sources and/or two or more studies from the healthcare research information. The one or more machine learning modules may use one or more machine learning techniques and/or deep learning techniques to correlate the types of research performed, results of research, numbers of patients and/or subjects used in research, whether research was performed in vitro and/or in silico, identities of one or more patients and/or subject used in the research, tests performed in the research, drugs and/or treatments given and/or performed in the research, and/or any other suitable data related to healthcare research between the two or more healthcare research information sources and/or two or more studies from the healthcare research information. The one or more machine learning modules may use the health information in performing the impact discovery analysis.

In some embodiments, platform 100 may be configured to analyze the health information using the one or more machine learning modules to determine gaps in care. The one or more machine learning modules may use the health information as training data set to determine the gaps in care. Gaps in care may include instances where healthcare professionals fail to prescribe one or more healthcare treatment routines according to established guidelines of best practice, and/or where healthcare professionals fail to perform correct testing of, treatment of, prescribing drugs to, and/or advisement of the patient and/or the population of patients, and/or any other suitable gap in healthcare by one or more healthcare professionals. In some embodiments, the platform 100 may be configured to correlate the health information related to the patient and/or the population of patients using the one or more machine learning modules. The one or more machine learning modules may apply one or more machine learning and/or deep learning techniques, such as Bayesian graphical networks, in correlating the health information to the patient and/or the population of patients. The one or more machine learning modules may correlate the health information to the patient and/or the population of patients to determine patterns related to the effects of the health information and/or portions of the health information to one or more health states of the patient and/or the population of patients, such as lifestyle, diagnosis, prognosis, present healthcare treatments, and previous healthcare treatments.

In some embodiments, the platform 100 may be configured to categorize the patient, the population of patients, and/or one or more patients included in the population of patients according to one or more of lifestyle, diagnosis and/or prognosis, social determinants of health, and present and/or previous healthcare treatments based on the one or more machine learning modules. In some embodiments, the one or more machine learning modules may employ one or more fuzzy rules in categorizing the patient, the population of patients, and/or the one or more patients included in the population of patients. In some embodiments, the one or more machine learning modules may apply one or both of a batch gradient descent and a stochastic gradient descent in categorizing the patient, the population of patients, and/or the one or more patients included in the population of patients.

In some embodiments, the platform 100 may be configured to receive health information related to a plurality of healthcare workers, each healthcare worker of the plurality of healthcare workers working as a team to treat at least one of the patient, the population of patients, and/or one or more patients included in the population of patients. In some embodiments, the health information may be related to a first plurality of healthcare workers and a second plurality of healthcare workers, the healthcare workers of the first plurality of healthcare workers working as a team to treat a first population of patients and the healthcare workers of the second plurality of healthcare workers working as a team to treat a second population of patients. In some embodiments, the platform 100 may be configured to share the health information related to the patient, the population of patients, the first population of patients, and/or the second population of patients with the first plurality of healthcare workers and/or the second plurality of healthcare workers to facilitate comprehensive sharing of information and collaborative treatment of one or both of the first and second populations of patients by one or both of the first and second pluralities of healthcare workers. The first and/or second populations of patients may be related by one or more similarities, such as similar lifestyles, diagnoses, prognoses, or any other suitable similarity. In some embodiments, the first and/or second populations of patients may be athletes competing in a sport or a plurality of sports. For example, the first and/or second populations of patients may be a sports team such as a football team, a soccer team, a baseball team, a cheer squad, a dance team, a gymnastics group, etc., or may be a group of patients diagnosed with an illness, such as diabetes, heart disease, influenza, SARS, or COVID-19. The first and/or second plurality of healthcare workers may be a diverse team of healthcare professionals, such as a team of healthcare professionals consisting of one or more nurses, disease experts, surgeons, physical therapists, and/or any other suitable type of healthcare worker.

In some embodiments, the platform 100 may be configured to create a digital twin of the first and/or second population of patients based on the health information related to the first or second population of patients, or both, using the digital twin module 1302 and/or the one or more machine learning modules. The digital twin of the first and/or second population of patients may be a customizable digital representation of one or more shared health states and/or health attributes of the first and/or second population of patients. For example, the first population of patients may consist of professional football players suffering from and/or prone to torn anterior crucial ligament injuries. The platform may create one or more digital twins of the football players to facilitate simulation, analysis, diagnosis, prognosis, and/or treatment of the football players based on information contained in the digital twin and/or simulations and/or predictions derived from the digital twin and the one or more machine learning modules. In some embodiments, the one or more machine learning modules may train based on the health information and/or the digital twins of the first and/or second population and may create one or machine learned models using one or more machine learning and/or deep learning techniques to anticipate one or more responses to medical treatment by the first and/or second population of patients.

In some embodiments, the platform 100 may be configured to facilitate volunteering for and/or opting into one or more treatment programs by the patient, one or more patients included in the first population of patients, and/or one or more patients included in the second population of patients. The platform 100 may use the healthcare research information, wherein the healthcare research information includes data regarding one or more research initiatives, clinical trials, experimental treatment programs, and/or other healthcare research programs, to correlate suitable patients with the one or more research initiatives, clinical trials, experimental treatment programs, and/or other healthcare research programs. The platform 100 may allow the suitable patients to opt into one or more of the healthcare research programs via the platform 100 and/or an interface thereof.

In some embodiments, the platform 100 may be configured to simulate the effects of the one or more research initiatives, clinical trials, experimental treatment programs, and/or other healthcare research programs, to correlate suitable patients with the one or more research initiatives, clinical trials, experimental treatment programs, and/or other healthcare research programs on the suitable patient by using, for example, machine learning, deep learning, and/or one or more digital twins of the patient to simulate the effects of the one or more research initiatives, clinical trials, experimental treatment programs, and/or other healthcare research programs, to correlate suitable patients with the one or more research initiatives, clinical trials, experimental treatment programs, and/or other healthcare research programs on the suitable patient prior to, during, or subsequent to opting into the research program by the suitable patient. In some embodiments, the platform 100 may compare simulations of one or more drugs and/or treatment options formed using the one or more machine learning modules and/or the digital twin module 1302, e.g., in silico simulations, to one or more results from one or more research programs having one or more in vivo and/or in vitro components. In some embodiments, the platform 100 may be configured to receive healthcare research information including one or more of methodology and results for one or more healthcare studies and compare the healthcare study information to simulations of one or more drugs and/or treatment options and effects thereof on the patient and/or the population of patients based on the one or more machine learning modules. The one or more machine learning modules may determine one or both of reliability and consistency of simulations performed using the platform 100, the one or more machine learning modules, and/or the digital twin module 1302 based on the comparisons of the healthcare study information to the simulations of the one or more drugs and/or treatment options.

In some embodiments, the platform 100 may analyze the health information and/or the healthcare research information to determine patients suitable for one or more healthcare research programs based on one or more of genetic, environmental, health state, and lifestyle properties of the patient, the population of patients, and/or one or more patients included in the population of patients using the one or more machine learning modules. For example, one or more of the healthcare research programs may require one or more patients having one or more particular genetic, environmental, health state, and/or lifestyle attributes, such as patients over the age of 40 of Eastern European descent diagnosed with Hodgkin's lymphoma, not being diabetic or prediabetic, and who do not exercise regularly. The platform 100 may calculate a desirability score of the patient, the population of patients, and/or one or more patients included in the population of patients, the desirability score being based at least partially on how closely the genetic, environmental, health state, and/or lifestyle properties of the patient, the population of patients, and/or one or more patients included in the population of patients fit criteria of suitable patients for the healthcare research program derived from the health information and/or the healthcare research information. In some embodiments, the platform 100 may determine similarities in one or more of the genetic, environmental, health state, and/or lifestyle properties of the patient, the population of patients, and/or one or more patients included in the population of patients to related results of one or more research programs and present the similarities to the user of the platform 100 and/or use the similarities to train the one or more machine learning modules using one or more machine learning and/or deep learning techniques.

In some embodiments, the platform 100 may receive sensor data from one or more environmental sensors, and/or wearable sensor worn by the patient, store the sensor data at the platform 100, and present the sensor data to the user of the platform 100. The data received from environmental sensor and/or wearable sensors may include one or both of biometric data and lifestyle data. The environmental sensor and/or wearable sensor may include one or more of sensor implemented on a smartphone, smart glasses, VR headsets, AR glasses, biometrics sensors, pacemakers, heartrate monitors, blood sugar sensor, or any other suitable type of environmental and/or wearable sensor. The platform 100 may implement the sensor data in the digital twin of the patient using the digital twin module 1302. The platform 100 may train the one or more machine learning modules using the sensor data according to one or more machine learning and/or deep learning techniques. In some embodiments, the environmental sensor and/or the one or more wearable sensor may be Internet of Things (IoT) sensors and may be in communication with one or more IoT communication devices, networks, and/or databases.

In some embodiments, the platform 100 may be configured to determine a personalized treatment plan for the patient based on at least one of the digital twin of the patient and the digital twin of the population of patients, the health information, the healthcare research information, and the sensor data using the one or more machine learning modules. By combining one or more of the digital twins of the patient and the digital twin of the population of patients, the health information, the healthcare research information, and the sensor data via the machine learning module, the machine learning module may formulate one or more very specific and precise personalized treatment plans particularly suited to the patient. The one or more personalized treatment plans may be based on one or more particular health states and/or attributes unique or substantially unique to the patient. The platform 100 may present the one or more personalized treatment plans to the user of the platform 100, thereby allowing the user, such as a healthcare professional, to enact the personalized treatment plan to provide personalized healthcare to the patient.

In some embodiments, the platform 100 may be configured to predict a response to a drug by the patient based on the genetic data of the patient derived from the health information. The one or more machine learning modules may use one or more machine learned models and/or the digital twin of the patient to simulate an effect of the drug on the body of the patient and/or one or more physiological systems and/or organs thereof.

In some embodiments, the platform 100 may facilitate consent for collaborative diagnosis and/or treatment by the patient and the population of patients based on similarities in one or more health states or other information derived from the health information related to the patient and the population of patients. The platform 100 may determine that the patient and the population of patients have one or more similarities, such as similar symptoms which may allow one or more healthcare professionals to provide more effective diagnosis and/or treatment if the one or more healthcare professionals are able to perform collaborative diagnosis and/or treatment on the patient and/or the population of patients, i.e., able to diagnose and/or treat the patient and patients included in the population of patients as a group rather than performing piecemeal diagnoses and/or treatments on each of the patient and the patients included in the population of patients. The platform 100 may prompt the patient and the patients included in the population of patients for consent. The one or more machine learning modules may determine when collaborative treatment may be suitable and/or ideal based on the health information.

In some embodiments, the platform 100 may be configured to assist in training of and or practicing by healthcare professionals, such as diagnosticians, by facilitating simulated diagnosis using the digital twin of the patient and/or the population of patients. The platform 100 may be configured to receive simulation instructions from the healthcare professionals, the simulation instructions being indicative of one or more potential treatment plans. The platform 100 may simulate the one or more treatment plans on the patient via the digital twin of the patient. The platform 100 may simulate application of best clinical practices for a desired clinical outcome on the patient via the digital twin of the patient. The platform 100 may evaluate efficacy of the one or more potential treatment plans via the digital twin of the patient and the simulation. In some embodiments, the platform 100 may calculate metrics related to differences between the one or more potential treatment plans, the best clinical practices, and outcomes thereof and present the metrics to the user of the platform 100. In some embodiments, the platform 100 may identify gaps in care attributable to one or more of the potential treatment plans and present the gaps in care to the user of the platform 100. For example, the platform 100 may present to a healthcare professional such as an oncologist a digital twin of a patient having symptoms of or having cancer. The oncologist may enter one or more potential treatment plans to the platform 100. The platform 100 may simulate effects and/or efficacy of each of the one or more potential treatment plans via the digital twin module 1302 and/or the one or more machine learning modules and present the effects and/or efficacy of each of the one or more potential treatment plans to the oncologist. The platform 100 may analyze the one or more potential treatment plans and compare the one or more potential treatment plans to best clinical practices, identify gaps in care according to the one or more potential treatment plans, and present one or both of the best clinical practices and gaps in care to the oncologist. The platform 100 may evaluate efficacy of the one or more potential treatment plans and present one or more efficacy metrics to the oncologist based on each of the one or more potential treatment plans.

In some embodiments, the platform 100 may be configured to assist in training of and or practicing by healthcare professionals by facilitating simulated prognosis using the digital twin of the patient and/or the population of patients. The platform 100 may be configured to receive simulation instructions from the healthcare professionals, the simulation instructions being indicative of one or more potential treatment plans. The platform 100 may simulate the one or more treatment plans on the population of patients via the digital twin of the population of patients. The platform 100 may simulate application of best clinical practices for a desired clinical outcome on the population of patients via the digital twin of the population of patients. The platform 100 may evaluate efficacy of the one or more potential treatment plans via the digital twin of the population of patients and the simulation. In some embodiments, the platform 100 may calculate metrics related to differences between the one or more potential treatment plans, the best clinical practices, and outcomes thereof and present the metrics to the user of the platform 100. In some embodiments, the platform 100 may identify gaps in care attributable to one or more of the potential treatment plans and present the gaps in care to the user of the platform 100.

In some embodiments, the platform 100 may be configured to assist in researching by healthcare researches, by facilitating simulated research via the digital twin of the patient and/or the population of patients. The platform 100 may be configured to receive simulation instructions from the healthcare researchers, the simulation instructions being indicative of one or more potential research regimens, such as clinical trials. The platform 100 may simulate the one or more research regimens on the patient via the digital twin of the patient. The platform 100 may simulate application of best clinical practices for a desired clinical outcome on the patient via the digital twin of the patient. The platform 100 may evaluate efficacy and/or results of the one or more potential research regimens using the digital twin of the patient and the simulation. In some embodiments, the platform 100 may calculate metrics related to differences between the one or more potential research regimens, the best clinical practices, and outcomes thereof and present the metrics to the user of the platform 100. In some embodiments, the platform 100 may identify gaps in care attributable to one or more of the potential research regimens and present the gaps in care to the user of the platform 100.

In some embodiments, the platform 100 may be configured to assist in researching by drug researches, by facilitating simulated drug research via the digital twin of the patient and/or the population of patients. The platform 100 may be configured to receive simulation instructions from the healthcare researchers, the simulation instructions being indicative of one or more potential drug prescriptions, such as clinical trials for one or more drugs. The platform 100 may simulate the one or more research regimens on the patient via the digital twin of the patient. The platform 100 may simulate application of best clinical practices for a desired clinical outcome on the patient via the digital twin of the patient. The platform 100 may evaluate efficacy and/or results of the one or more potential research regimens using the digital twin of the patient and the simulation. In some embodiments, the platform 100 may calculate metrics related to differences between the one or more potential research regimens, the best clinical practices, and outcomes thereof and present the metrics to the user of the platform 100. In some embodiments, the platform 100 may identify gaps in care attributable to one or more of the potential research regimens and present the gaps in care to the user of the platform 100.

In some embodiments, the platform 100 may receive investment data related to money spent on one or more of research programs, treatment regimens, and costs of care by one or more healthcare providers, healthcare researchers, and health insurance providers. The platform may determine a return on investment metric based on the investment data. The return on investment metric may be indicative of an amount of money invested versus an amount of money recovered and/or costs of care by one or more of the healthcare provider, the healthcare researcher, and the health insurance provider. In some embodiments, the platform 100 may analyze a first treatment plan and a second treatment plan and determine whether providing the first treatment plan to the patient and/or the population of patients may result in an improved return on investment metric versus providing the second treatment plan to the patient and/or the population of patients. In some embodiments, the platform 100 may receive data related to one or more pre-existing conditions of the patient and/or the population of patients using the health information. The platform 100 may determine an effect on the pre-existing condition on the return on investment metric of the patient and/or the population of patients. The one or more machine learning modules may train using the investment data using one or more machine learning and/or deep learning techniques and form one or more models and/or predictions related to the return on investment metric. For example, when administering a treatment program, performing healthcare research, and through the course of insuring a patient, healthcare providers, healthcare researchers, and healthcare insurers invest capital and keep records of capital invested. The platform 100 may compare capital invested by each of the healthcare providers, healthcare researches, and healthcare insurers to capital gained in administering a treatment program, performing healthcare research, and through the course of insuring a patient, and use the comparison to determine the return on investment metric. The return on investment metric may be used by the healthcare provider to set costs of healthcare, by the healthcare researcher to determine how much money should be spent on one or more research programs to make a desired return on investment, and by the healthcare investor to determine how to configure healthcare insurance plans and set insurance rates to make a desired return on investment.

In embodiments, the pharmacological tracking platform 100, as described herein, may include a health monitoring command center module that allows an organization to monitor, respond to, and manage outbreaks that may potentially impact its employees, its business operations, and its market, including geographic markets of interest. The Covid-19 (hereinafter also referred to as “Covid” “CV19” or “CV-19”) pandemic has presented unprecedented challenges for organizations. Decisions that an organization previously need not consider, such as the health status of employees' neighborhoods, may now be crucial business priorities for determining whether and when a business may reopen, which employees may safely return to work, who should quarantine and for how long, and so forth. Complicating an organization's tasks further is the fact that information available regarding testing, community public health and the like may be imprecise and subject to rapid change. A neighborhood one week may record zero positive Covid-19 cases but become a “hotspot” of infection the next week. Variances in testing capacity, testing turn times and other factors may all impact the availability of information and the timing of needed updates for organizations to have intelligent planning. For example, Covid symptoms may not appear for as many as 12 days after acquiring the virus, meaning that employees, or others, with new infections may not know they are spreading the virus. Further, the presence of Covid antibodies may not imply immunity and Covid tests may be rife with false-positives and false-negatives, depending on the make of the test, which has far reaching implications for managing test result data. Thus, employers and organizations must develop and manage their return-to-work and stay-at-work guidelines, including testing mandates, workplace policies, and policies surrounding social considerations such as contact tracing.

Key among an employer's concerns may be issues such as: What portion of the workforce has a positive Covid virus test result and an active illness? What portion of the workforce has tested positive for the Covid antibody? Which employees have symptoms? Which employees are due for a retest and when must those tests be completed to remain compliant? Which employees have been exposed to an infected colleague and how broad was the exposure? How many employees are available for work? Which employees have provided consent to share test results? Do any employees live in hot zones where there may be a higher chance of bringing the virus to work? As described herein, the health monitoring command center and associated platform 100 facilitate the collection and presentation of data, data models, community health summaries and risk measures, to assist in answering these key concerns and providing guidance and recommendations on next steps an organization might take to mitigate the risks presented in their employee or personnel population of interest.

In an example, organizations' responses to the Covid pandemic may include developing approaches to lab testing of personnel, checking the symptoms of personnel, implementing social guidelines (e.g., social distancing of at least six feet between persons), and permitting/restricting access to certain physical domains of a facility to minimize social contact. For example, as regards to lab testing, an organization may require that all employees returning to a facility first get a self-assessment with potentially further testing based on their responses and self-reported symptoms, for example further testing for presence of the Covid-19 virus or Covid antibodies (Ab). An organization may need to establish a medically reasonable guideline, and informed, voluntary consent procedure, for periodic testing of the non-Ab positive population for presence of the virus. Verified test results may guide decision making and assist in advising persons who may best quarantine and consider, for those exposed, whether contact tracing may be appropriate. An organization may also need to establish ongoing symptoms checking. For example, contactless temperature checks may be performed upon personnel entering and leaving a facility, regular questioning may be made regarding the presence of symptoms. An organization may also need to consider and monitor checks of geographic or other risk areas related to the employee's residence and work site (e.g., the rate of infection in an employee's zip code and local risk, the presence of an infected family member in the employee's home, and so forth). Physical measures must also be taken and monitored by an organization, such as social distancing, mandatory personal protective equipment (PPE) in places where proximity to others is likely, intensive preventive hygiene regimen, routine disinfectant protocols, as well as signage to reinforce new behavior and hygiene policies. Where practical, an organization may also minimize non-essential travel between floors, departments, buildings; minimize shared space, and/or equipment, and establish physical and/or virtual checkpoints to reinforce essential passage areas.

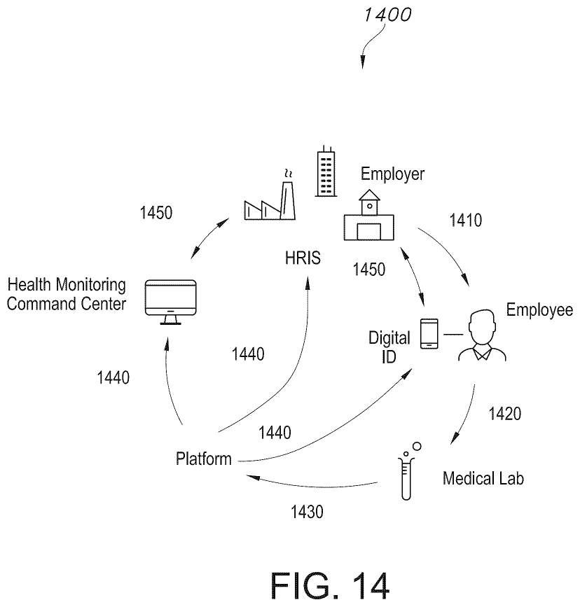

In embodiments, the platform 100, as described herein, may include a centralized command and control tool (hereinafter also referred to as a “health monitoring command center” module of the platform 100) for organizations to monitor adherence to organization testing mandates, understand operational readiness levels and risks down to specific workplace areas, and communicate with external sources such as electronic medical records (EMRs), public health agencies, insurer databases, pharmacy databases, testing lab databases, businesses, companies and/or organizations, as well as other computing device(s), systems, data sources, applications, and platforms, via a network 380. FIG. 14 depicts a simplified view of the health monitoring command center module and the platform 100 in relation to an employer and its employee (and corresponding digital identifier (ID), a medical lab and a general human resource information system (HRIS). In this simplified diagram, methodologies are outlined and labeled. At 1410, an employer is depicted as adopting a return-to-work (RTW) testing strategy and mandating employee testing. At 1420, employees get tested by a contracted medical lab which, at 1430, communicates lab results and associated data directly to the platform 100, as described herein. At 1440, the platform 100 pushes the lab-reported test results to the health monitoring command center (and its associated dashboard), an HRIS, and to a digital ID that is associated with the employee for which the lab test(s) apply. at 1450, an employer monitoring adherence to policy and assessing employee population using the health monitoring command center, as described herein.

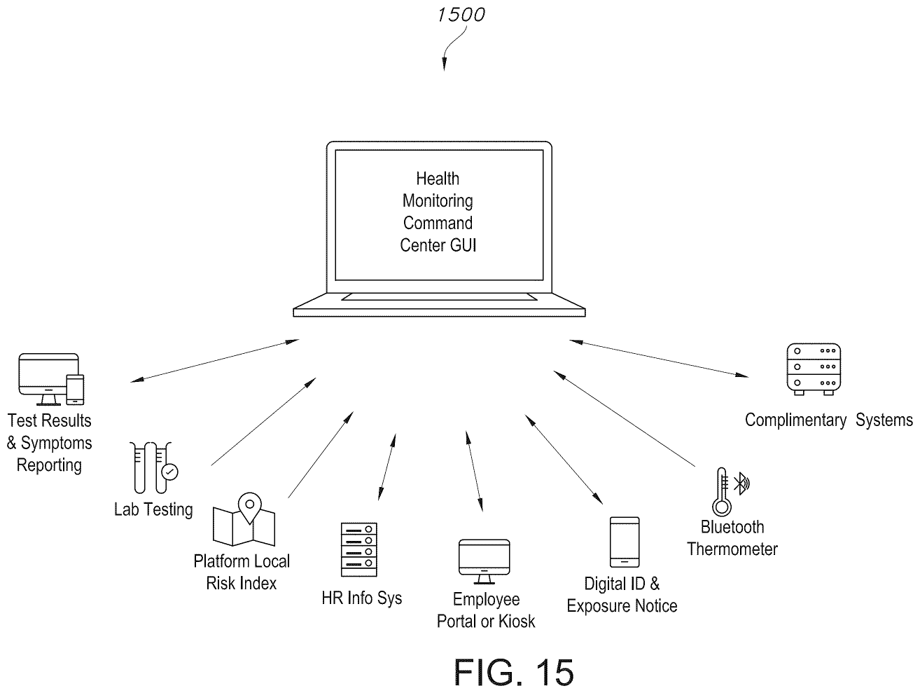

As shown in FIG. 15, the health monitoring command center may present information to a user, such as an employer, including, but not limited to, test results and symptoms reported, lab testing data, measures of risk and summary scoring of risk, such as a local risk index (LRI), as described herein, human resources data, digital IDs of employees, device data, such as a Bluetooth-connected thermometer, or some other complementary system or platform, such as a medical record database or some other type of data or medical platform. The health monitoring command center may be further associated with an employee portal or kiosk through which employees access their personal information and/or information related to their employer and its facilities and programs. In embodiments, the digital ID may be further associated with additional medical records and data including, but not limited to, a vaccination history. For example, a student that is required to present proof of certain vaccinations may be able to access and document such vaccination history using the health monitoring command center and their associated digital ID.

In embodiments, the health monitoring command center of the platform 100 may present medical lab testing results imported directly from the lab into the health monitoring command center, allowing employers to monitor employees against the company's testing policies. Test results and employee status may be displayed by employee and workplace location (office, department, floor, building, region, company, etc.). An LRI may be calculated and presented to inform employers of Covid, or other testing trends at the community level to assist the employer's decision-making regarding return-to-work or other processes. An LRI may provide detailed “community level” views of testing trends to better inform local decision making based on an aggregated and de-identified view of near-real time Covid and Covid Ab test, or other test results from a coalition of laboratories. The LRI may visualize a risk trend using test results from a user-specified prior time period (e.g., the prior 7 days, prior month, and so on). The LRI may also be used to validate and/or compare a measure of local risk against a separate source of data (e.g., public health data and statistics released by state or county authorities). Because data in any outbreak will have bias and error, this additional validation step may enable more accurate decision making regarding needed risk minimization steps and ultimately benefit the health of a population, such as employees of a work site. The LRI may also be used to establish a baseline measure of health status of a work site, community or other regions. This may allow for monitoring of changing health conditions in a region, such as the neighborhood in which a given employee resides. The LRI may also inform when certain public health thresholds are crossed, for example, it is generally considered that local containment measures for Covid infection prevention are generally only effective if the prevalence in the community is less than 1%. In embodiments, the LRI may allow the calculation of risk based at least in part on the calculation of risk based on moving time periods and allow for geographic analysis at a more detailed level than is typically made available, thus mitigating the risk inherent in some health statistics reporting of “averaging the averages.” More geo-specific LRI data will allow for more geo-specific decision making.

The health monitoring command center may generate automatic alerts, email push notifications, or some other notice type, which may be established by a user setting and/or event- or metric-triggered. Role-based access to the health monitoring command center may enhance employee privacy and limit the visibility of private information to those with need-to-know access and permission. As a result of this privacy-centric functioning of the health monitoring command center, employees may remain in control of their health information by choosing/consenting to share their information. The health monitoring command center and platform 100 may accept individual Covid, or other, test results in real time directly from the performing medical lab, as described herein. This may provide digital badging, proof of test status for both the antibody test or the virus test, as well as self-reported symptom history, or some other data type. A daily symptoms diary may be included for capturing and reporting individual symptoms and possible exposure risks and provide recommendations and contact tracing should it be necessary using a safe, secure, anonymized reporting engine available through both iOS and Android using BLE technology associated with the health monitoring command center. The health monitoring command center may also interact and communicate with other platforms and shared applications, such as those of airlines, restaurants, or some other industry.

In embodiments, the platform 100 and health monitoring command center may collect data, such as lab test results, to determine a health status for a patient, such as a positive or negative test result on a Covid test. In embodiments, the platform 100 may obtain data relating to the patient, such as demographic data including but not limited to household and location data, historical data, health status data, employment data, or some other type of demographic data, as well as prior test results including lab tests associated with other patients that may be matched by some criterion, attributes of those patients (e.g., age, sex, weight, body type), and the result of the treatment (e.g., quarantine timing and duration, prescriptions and the like). In this way, the patient may be flagged for monitoring, follow-up, or some other action by a healthcare provider, employer, or some other party. Furthermore, in embodiments, the platform 100 may make recommendations based at least in part on one or more different tests for the patient during or after the treatment, and based on external data accessed by the platform, for example, community public health data regarding Covid infection rates by geographic area.

In embodiments, the platform 100 and health monitoring command center may monitor test results of a plurality of subjects, such as employees of an organization, to determine whether the respective subjects have, or are at greater relative risk of having, a disease state of interest, such as being positive for Covid. The health monitoring command center may provide notifications and/or recommendations to appropriate third parties, such as employers, healthcare organizations (e.g., hospitals and/or clinics), long term care facilities, physicians, pharmacies, insurers, corrections facilities, first responders, government organizations, universities and schools, travelers and those in the travel industry (e.g., airlines or hotels), consumers, or some other third party. Within an organization, the platform 100 and health monitoring command center may utilize customer relationship management data and capabilities of the organization, whereby the health monitoring command center may leverage these capabilities to provide the notifications and/or recommendations regarding employees health states, such as current Covid test status, and recommended next steps for that employee and/or other employees and personnel the employee may have come in contact with.

In embodiments, the platform 100 and health monitoring command center may include a customer relationship management (CRM) system 102, a test management system 104, a prescription monitoring system 106, a machine learning system 108, and/or a workplace advisor system. The health monitoring command center and its associated dashboard (i.e., graphic user interface), may allow an organization to identify, test, monitor and track employees related to a health status of interest.

In embodiments, the test management system 104 may determine whether to recommend lab testing for a person given a current status of the person. In response to the test management system 104 determining to recommend lab testing, the CRM system 102 may provide a mechanism (e.g., a GUI) by which a user (e.g., a representative of a lab testing organization) may provide the notification recommending lab testing to a healthcare provider (e.g., the treating physician or the office thereof), a pharmacy, and/or an insurance provider. The test management system 104 may provide other features as well, such as quality assessment relating to testing labs. Test results may be verified and uploaded directly from participating laboratories to a secure mobile solution that links the employee identity to their patient profile. A digital token may act as a badge for employees based on their test results status, and, in an example, QR-code scanning may indicate compliance with the testing protocol and determines employee eligibility for work. Official government and/or employee-issued ID's may be incorporated to verify identities at a time of testing, and test result information may be shared by the employee in a private, consent-driven, touchless transaction. The health monitoring command center may further enable and incorporate contact tracing should it be necessary.

In embodiments, the CRM system 102 may be accessed by users associated with a testing lab system 150. In embodiments, the CRM system 102 may allow these users to manage relationships and communications with healthcare providers associated with healthcare systems 130, pharmacy employees associated with pharmacy systems 140, insurance providers associated with insurance systems 160, organizations and/or employers. In embodiments, the CRM system 102 may receive recommendations and/or notifications from the test management system 104 and/or the prescription monitoring system 106. The CRM system 102 may perform additional or alternative tasks, such as obtaining data from external data sources (e.g., healthcare systems 130, pharmacy systems 140, testing lab systems 150, and/or insurance system 160) and may structure the obtained data into different types of records according to respective schemas, and present such data and its derivatives to the health monitoring command center.

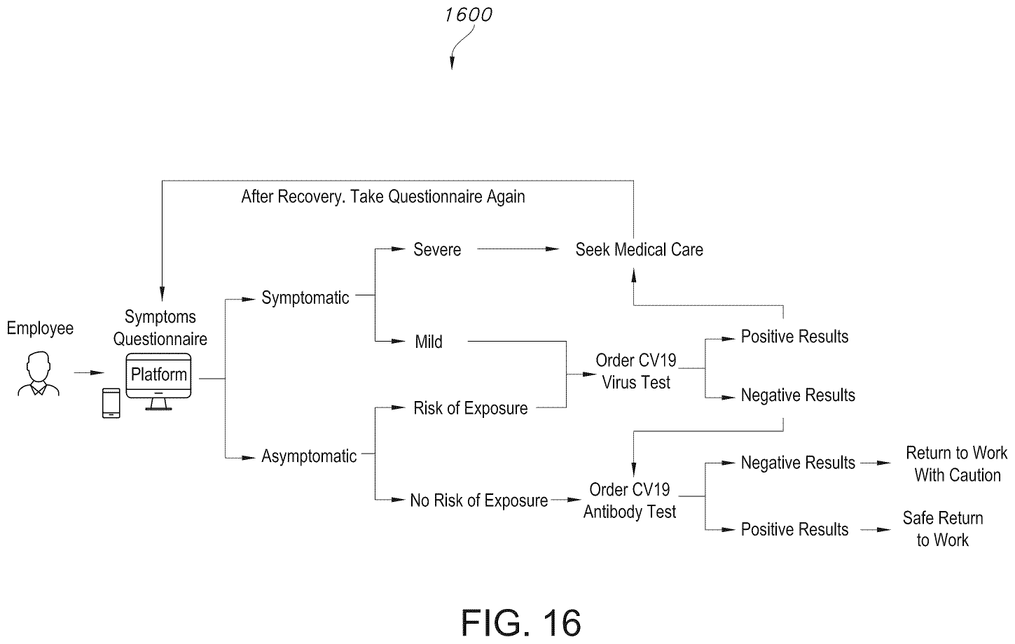

FIG. 16 depicts a simplified example workflow of an employee's interaction with the health monitoring command center and a possible sequence of steps involved. To begin, an employee may be presented a symptom questionnaire to complete related to Covid symptoms, or symptoms corresponding to another health state of interest (e.g., influenza, measles, or some other illness or health state). If the employee is symptomatic, they may be directed to seek immediate health care if the symptoms are severe, or to receive a Covid, or other tests if the symptoms are mild. If the employee is asymptomatic but has a higher relative risk of exposure (e.g., living within a neighborhood having a relatively high LRI), they may also be directed to receive a Covid, or other test. If the asymptomatic employee has a relatively low level of risk of exposure they may be directed to receive a Covid Ab, or other test. If that test is negative they may be cleared to return to work with caution (e.g., with ongoing periodic monitoring). If the Covid Ab test is positive, the employee may be cleared to return to work without additional monitoring planned.

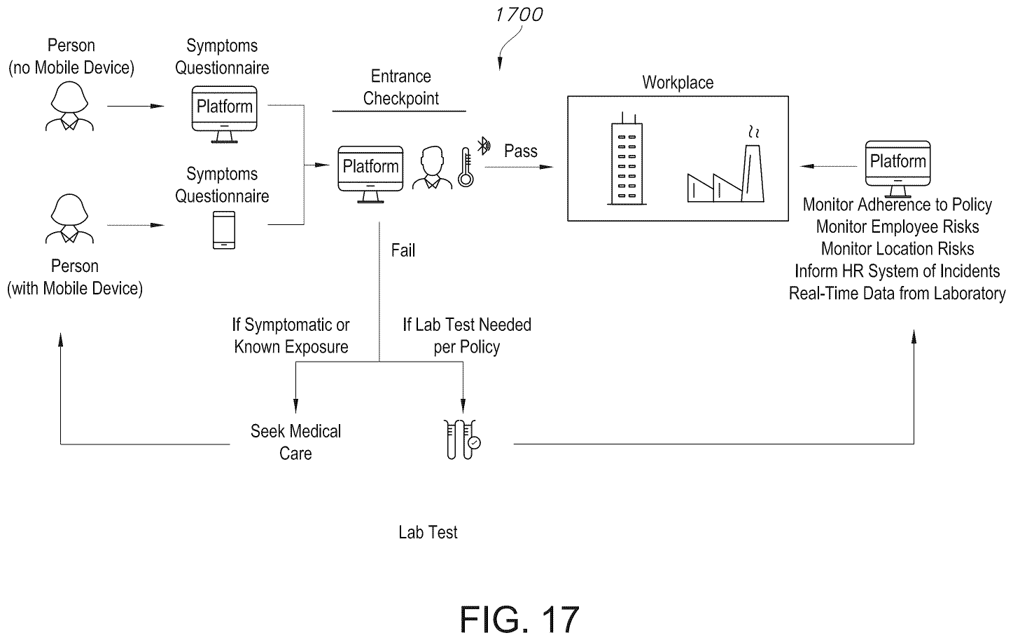

As shown in FIG. 17, a person, such as an employee, may interact with the health monitoring command center via a computing device, application running on a mobile device (e.g., a smart phone, tablet, or other mobile computing device) and complete a symptom questionnaire at an entrance checkpoint to a workplace. Based on the results of this symptom questionnaire, the asymptomatic employee may be cleared to enter the workplace and the symptomatic employee may be directed to seek immediate medical care (e.g., if a known exposure to Covid, or some other illness of interest, occurred) and/or to receive a lab test, such as a Covid test. The medical lab performing the Covid or other test may report the test results directly back to the platform 100 and health monitoring command center, as described herein, and the employer in charge of the workplace may monitor the results for a plurality of employees in real time. This information may be further shared with other platforms including, but not limited to, HRIS or other platforms.

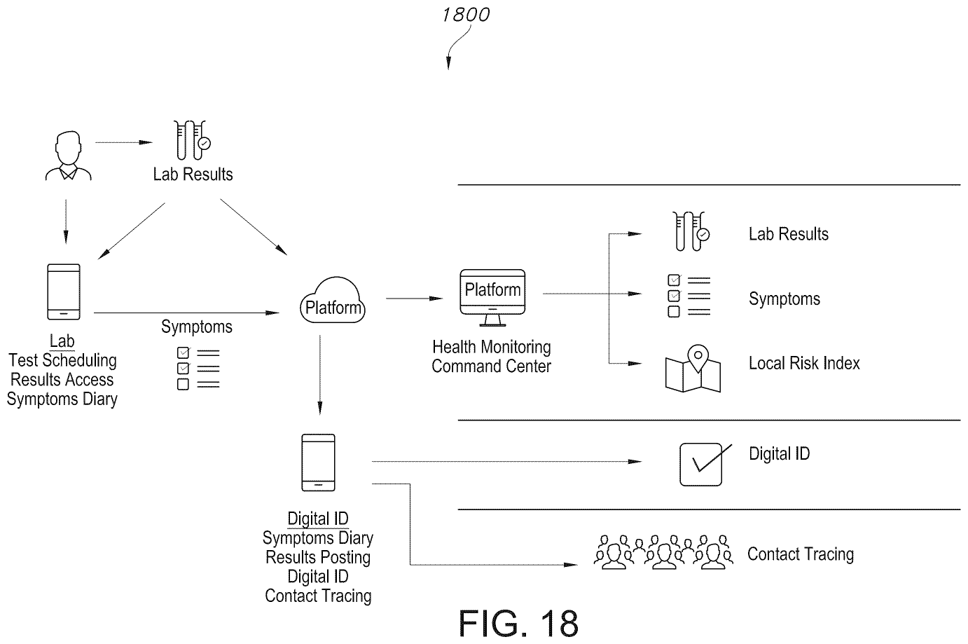

In embodiments, as shown in FIG. 18, the symptoms summary, lab results, and LRI that is associated with an individual may be tracked by the individual using a mobile application in communication with the health monitoring command center. Using the health monitoring command center, an employee, employer or other party may continuously monitor lab results, symptoms, LRI and other data and detect and measure trends, such as a worsening Covid infection or other rate. Using the digital ID that is associated with an individual, such as an employee, and stored by the health monitoring command center, the health monitoring command center may also track the relationships among digital IDs (e.g., co-workers sharing an office space) which may in turn facilitate activities such as contact tracing to determine who an infected employee may have come in contact with and exposed to the virus. This may facilitate an employer's quarantine policy and help minimize the duration that potentially exposed employees or personnel are permitting on a work site. The digital ID of the health monitoring command center may also give individuals custody of their own lab results and may be portable and used with a plurality of third parties whom the individual consents to share testing or other data with. For example, some facilities, business or groups may require documentation that an individual is illness free, or offer incentives (e.g., price incentives to persons willing to share testing or other data), such as airlines, hotels, entertainment facilities, or others.

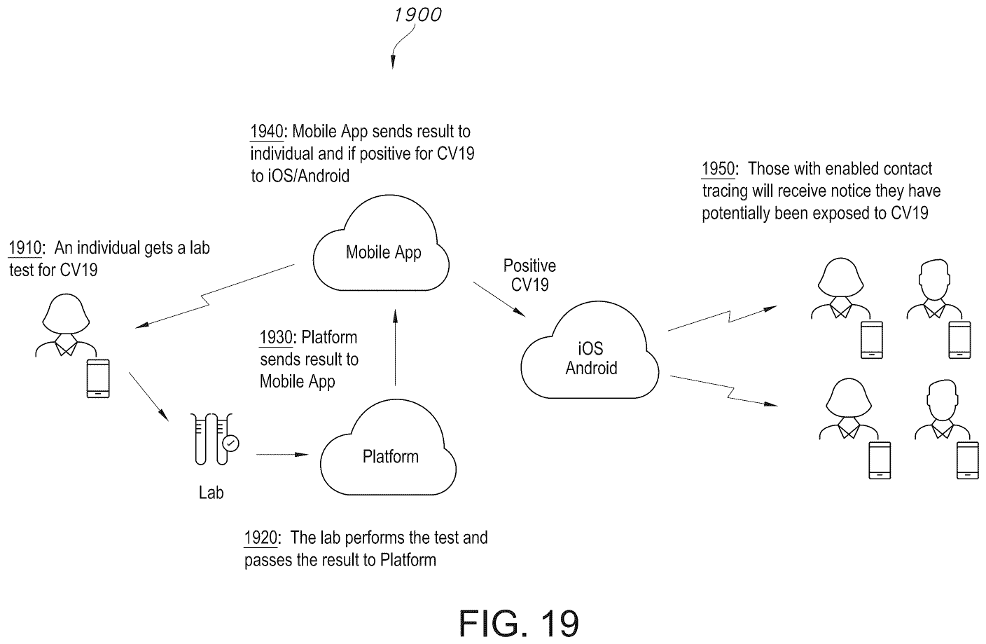

A simplified example of contact tracing using the health monitoring command center is shown in FIG. 19. At 1910, an individual, such as an employee, is tested for Covid. The medical lab performing the testing may, at 1920, communicate the individual's test result to the health monitoring command center. If the test result is positive, the health monitoring command center may send a notification back to the individual using the mobile application, or other means as described herein, informing her that she should quarantine, receive medical help or provide some other information and/or instruction. The health monitoring command center may also notify others who were recently in contact (or possible contact) with the person testing positive for Covid, and an employer or other interested party, and provide information and/or instructions on advised next steps. Such information communicated may be anonymized by the health monitoring command center to ensure employee/patient privacy.

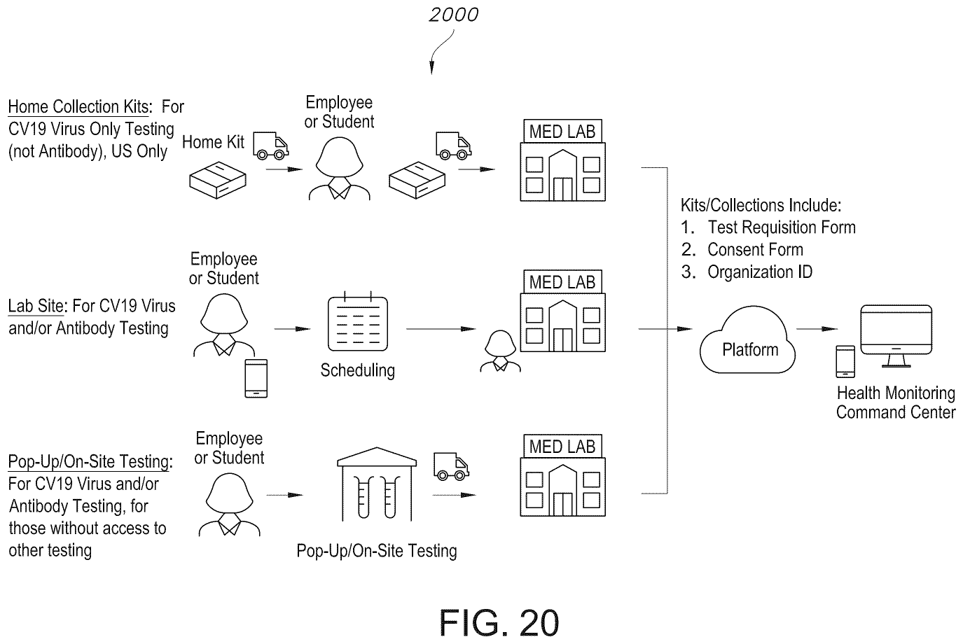

In embodiments, as shown in FIG. 20, the health monitoring command center may receive test results and other data from a plurality of testing sites and types, for example, a home collection kit sent by an individual directly to a medical lab, a lab site, such as within a physician's clinic, or a pop-up testing site, such as a community-based temporary testing site. Such testing kits and collections may include a test requisition form, patient consent form, organization ID, or some other type of health or other information that is communicated to the health monitoring command center and platform 100.

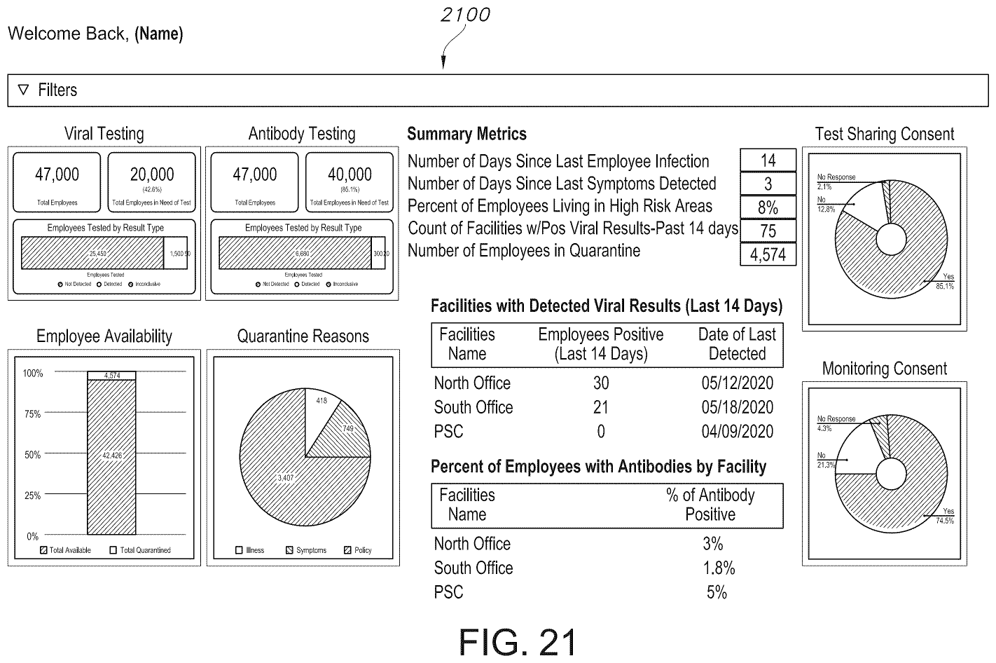

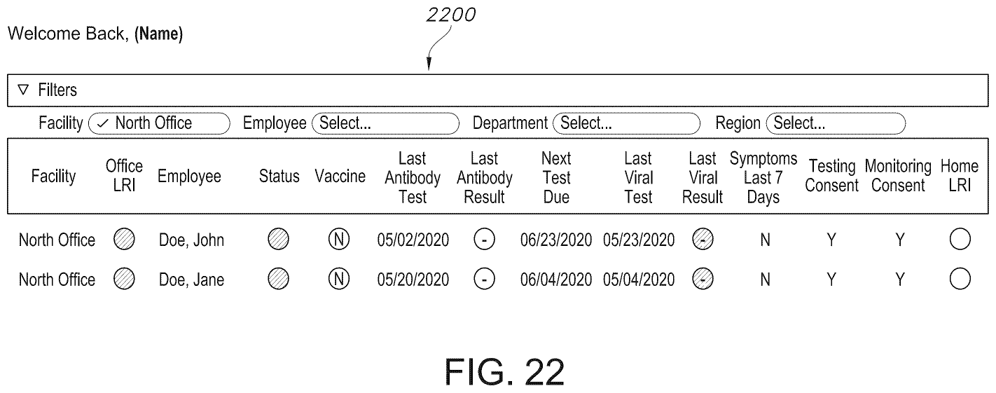

FIG. 21 presents a hypothetical dashboard of the health monitoring command center displaying the results for a particular work site. The dashboard of the health monitoring command center may present a plurality of testing, health status, community or other data including, but not limited to, viral testing results, antibody testing results, and data on an employees' availability and quarantine status. Summary metrics may be provided covering at least the number of days since the last infection or symptom was detected, the percentage of employees living in high risk areas (e.g., as measured by the LRIs), the count of an organization's facilities that have or have had positive test results for Covid or some other health condition, and the number of employees in quarantine. The health monitoring command center may provide additional detail regarding those facilities in particular at which at least one employee has tested positive for a health state of interest, like Covid, and the percentage of employees that have detectable Covid antibodies, or some other biologic marker of interest. The health monitoring command center may also provide data for managing employee consent and privacy measures. FIG. 22 shows an example dashboard view of an employee roster and the corresponding health status indicators of interest, such as the organization's facility, office LRI, employee name, health state status (e.g., positive/negative for Covid), whether the employee has received a vaccine for a specified illness, the date of the last antibody test, the next planned test date, the date of the last viral test, the last viral test result (positive/negative), the presence of any symptoms with the employee within the last specified time period of interest, whether consent for testing was obtained/monitored from the person, and what the LRI is for the person's home environment. The LRI may also allow risk assessments and analysis for regions in which an organization does business, or through which a person plans to travel. For example, a company, such as a delivery company, that has a fleet of trucks and employees spread across a broad geographic region may be able to monitor the LRI of the communities in which the trucks/employees operate to determine which trucks/employees may need to implement additional protective measures due to a higher relative infection rate in the region(s) in which they operate.

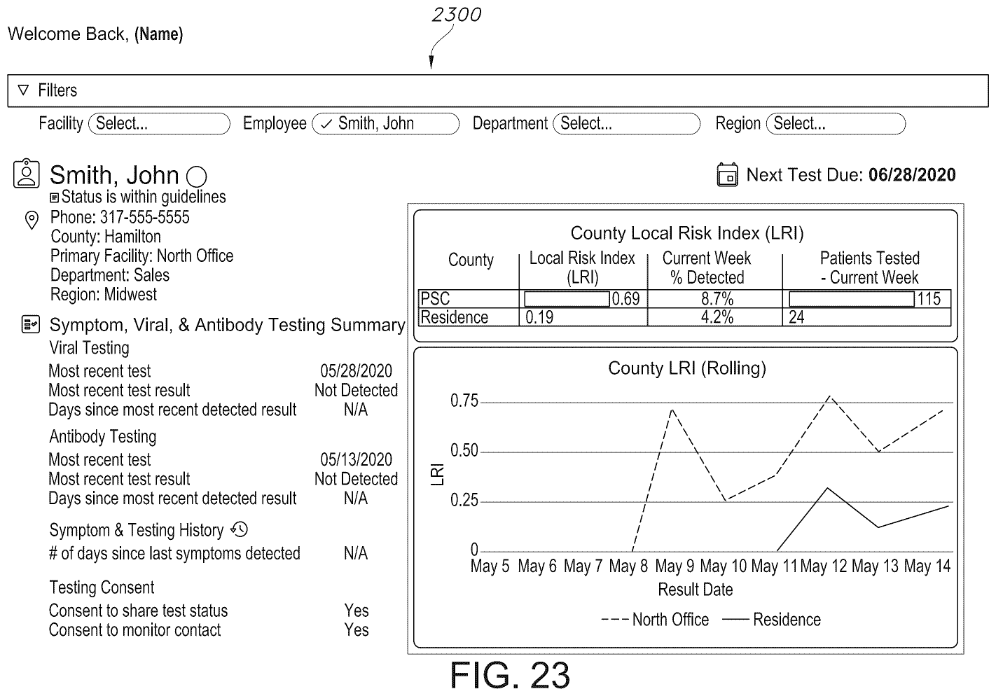

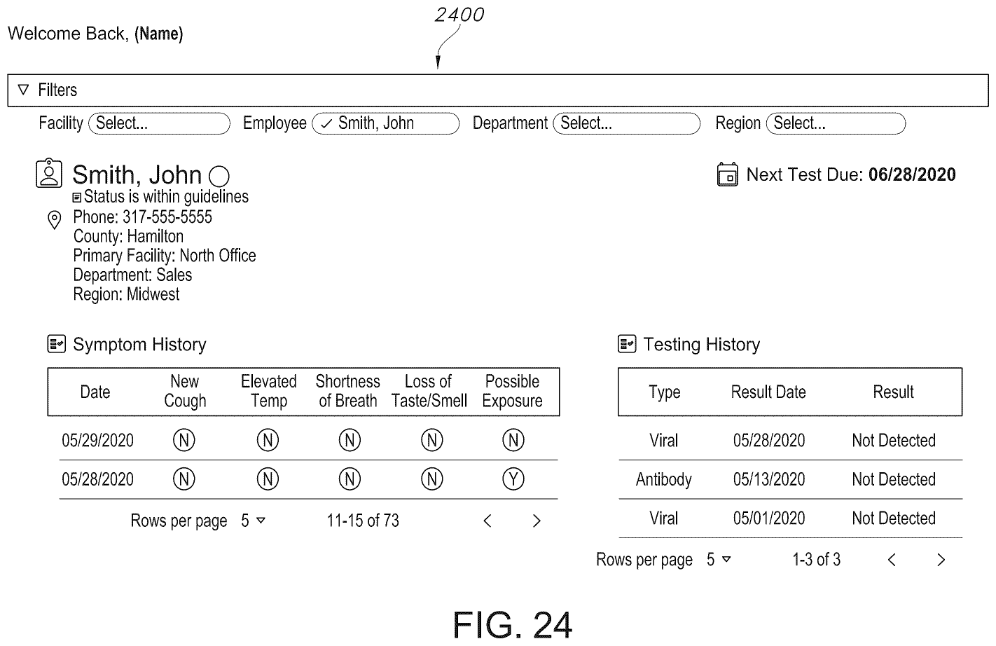

In embodiments, as shown in FIG. 23, the health monitoring command center may present a dashboard view of an individual's summary data including, but not limited to, demographic data, symptom, viral, and antibody testing summaries, and a longitudinal view of the LRI for a region of interest associated with the individual, such as the LRI for the county in which the individual lives. The health monitoring command center may allow a user to “drill down” into aspects of the data presented by selecting a data domain for which the health monitoring command center will present more detailed information. For example, FIG. 24 depicts a simplified view of a person's detailed symptom and testing history, indicating the dates on which symptoms and/or testing events occurred and the results of each occurrence. Additional types of data and information that may be summarized in the dashboard of the health monitoring command center include, but are not limited to: Percent employees tested (virus and antibody) by employer workplace categories; Percent/number of employees in quarantine by employer workplace categories; Percent of employees unable to come to work (symptoms, infection, guidelines); Number of days since last infection detected by employer workplace categories; Number of days since last symptoms detected by employer workplace categories; Offices and facilities LRI (County/PUMA); Employee risk average per office based on individuals LRI (County/PUMA); Percent of employees living in high LRI (County/PUMA); Percent employees giving consent for sharing test status by employer workplace categories; Percent employees consent driven contact tracing enabled by employer workplace categories, or some other type of data presentation.

In embodiments, the health monitoring command center and platform 100 may provide insights for screening and monitoring persons is a plurality of business types and environments including, but not limited to: Students in pre-K-12, universities, dormitories; Residents in long term care centers; Visitors to hospitals; Passengers on airplanes, cruise lines, public transit, trains, buses; Travelers in hotels, taxis and the like; Shoppers in retail stores; Fans at sporting events; Attendees at conferences; Congregations at worship, or some other business type or environment.

Detailed embodiments of the present disclosure are disclosed herein; however, it is to be understood that the disclosed embodiments are merely exemplary of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The terms “a” or “an,” as used herein, are defined as one or more than one. The term “another,” as used herein, is defined as at least a second or more. The terms “including” and/or “having,” as used herein, are defined as comprising (i.e., open transition).

While only a few embodiments of the present disclosure have been shown and described, it will be obvious to those skilled in the art that many changes and modifications may be made thereunto without departing from the spirit and scope of the present disclosure as described in the following claims. All patent applications and patents, both foreign and domestic, and all other publications referenced herein are incorporated herein in their entireties to the full extent permitted by law.

The methods and systems described herein may be deployed in part or in whole through a machine that executes computer software, program codes, and/or instructions on a processor. The present disclosure may be implemented as a method on the machine, as a system or apparatus as part of or in relation to the machine, or as a computer program product embodied in a computer readable medium executing on one or more of the machines. In embodiments, the processor may be part of a server, cloud server, client, network infrastructure, mobile computing platform, stationary computing platform, or other computing platforms. A processor may be any kind of computational or processing device capable of executing program instructions, codes, binary instructions and the like, including a central processing unit (CPU), a general processing unit (GPU), a logic board, a chip (e.g., a graphics chip, a video processing chip, a data compression chip, or the like), a chipset, a controller, a system-on-chip (e.g., an RF system on chip, an AI system on chip, a video processing system on chip, or others), an integrated circuit, an application specific integrated circuit (ASIC), a field programmable gate array (FPGA), an approximate computing processor, a quantum computing processor, a parallel computing processor, a neural network processor, or other type of processor. The processor may be or may include a signal processor, digital processor, data processor, embedded processor, microprocessor or any variant such as a co-processor (math co-processor, graphic co-processor, communication co-processor, video co-processor, AI co-processor, and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor, or any machine utilizing one, may include non-transitory memory that stores methods, codes, instructions and programs as described herein and elsewhere. The processor may access a non-transitory storage medium through an interface that may store methods, codes, and instructions as described herein and elsewhere. The storage medium associated with the processor for storing methods, programs, codes, program instructions or other types of instructions capable of being executed by the computing or processing device may include but may not be limited to one or more of a CD-ROM, DVD, memory, hard disk, flash drive, RAM, ROM, cache, network-attached storage, server-based storage, and the like.

A processor may include one or more cores that may enhance speed and performance of a multiprocessor. In embodiments, the process may be a dual core processor, quad core processors, other chip-level multiprocessor and the like that combine two or more independent cores (sometimes called a die).

The methods and systems described herein may be deployed in part or in whole through a machine that executes computer software on a server, client, firewall, gateway, hub, router, switch, infrastructure-as-a-service, platform-as-a-service, or other such computer and/or networking hardware or system. The software may be associated with a server that may include a file server, print server, domain server, internet server, intranet server, cloud server, infrastructure-as-a-service server, platform-as-a-service server, web server, and other variants such as secondary server, host server, distributed server, failover server, backup server, server farm, and the like. The server may include one or more of memories, processors, computer readable media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, social networks, and the like. Additionally, this coupling and/or connection may facilitate remote execution of programs across the network. The networking of some or all of these devices may facilitate parallel processing of a program or method at one or more locations without deviating from the scope of the disclosure. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs, or codes as described herein and elsewhere may be executed by the client. In addition, other devices required for the execution of methods as described in this application may be considered as a part of the infrastructure associated with the client.

The client may provide an interface to other devices including, without limitation, servers, other clients, printers, database servers, print servers, file servers, communication servers, distributed servers, and the like. Additionally, this coupling and/or connection may facilitate remote execution of programs across the network. The networking of some or all of these devices may facilitate parallel processing of a program or method at one or more locations without deviating from the scope of the disclosure. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements. The methods and systems described herein may be adapted for use with any kind of private, community, or hybrid cloud computing network or cloud computing environment, including those which involve features of software as a service (Saas), platform as a service (PaaS), and/or infrastructure as a service (IaaS).

The methods, program codes, and instructions described herein and elsewhere may be implemented on a cellular network with multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, 4G, 5G, LTE, EVDO, mesh, or other network types.

The methods, program codes, and instructions described herein and elsewhere may be implemented on or through mobile devices. The mobile devices may include navigation devices, cell phones, mobile phones, mobile personal digital assistants, laptops, palmtops, netbooks, pagers, electronic book readers, music players, and the like. These devices may include, apart from other components, a storage medium such as flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer-to-peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g., USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, network-attached storage, network storage, NVME-accessible storage, PCIE connected storage, distributed storage, and the like.

The methods and systems described herein may transform physical and/or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable code using a processor capable of executing program instructions stored thereon as a monolithic software structure, as standalone software modules, or as modules that employ external routines, code, services, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices, artificial intelligence, computing devices, networking equipment, servers, routers and the like. Furthermore, the elements depicted in the flow chart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps associated therewith, may be realized in hardware, software or any combination of hardware and software suitable for a particular application. The hardware may include a general-purpose computer and/or dedicated computing device or specific computing device or particular aspect or component of a specific computing device. The processes may be realized in one or more microprocessors, microcontrollers, embedded microcontrollers, programmable digital signal processors or other programmable devices, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine-readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions. Computer software may employ virtualization, virtual machines, containers, and other capabilities.

Thus, in one aspect, methods described above and combinations thereof may be embodied in computer executable code that, when executing on one or more computing devices, performs the steps thereof. In another aspect, the methods may be embodied in systems that perform the steps thereof and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

The use of the terms “a” and “an” and “the” and similar referents in the context of describing the disclosure (especially in the context of the following claims) is to be construed to cover both the singular and the plural, unless otherwise indicated herein or clearly contradicted by context. The terms “comprising,” “with,” “including,” and “containing” are to be construed as open-ended terms (i.e., meaning “including, but not limited to,”) unless otherwise noted. Recitations of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. The term “set” may include a set with a single member. No language in the specification should be construed as indicating any non-claimed element as essential to the practice of the disclosure.

While the foregoing written description enables one skilled to make and use what is considered presently to be the best mode thereof, those skilled in the art will understand and appreciate the existence of variations, combinations, and equivalents of the specific embodiment, method, and examples herein. The disclosure should therefore not be limited by the above described embodiment, method, and examples, but by all embodiments and methods within the scope and spirit of the disclosure.

All documents referenced herein are hereby incorporated by reference as if fully set forth herein.

At least some aspects of the present disclosure will now be described with reference to the following numbered clauses.

### Set A—Exemplary Clauses

1. A computerized method for healthcare data management, the method comprising:
   receiving, at a healthcare data system computing device including one or more processors, health information from one or more healthcare communication sources, the health information including data related to an individual patient and data related to a population of patients;
   storing, using the healthcare data system computing device, the health information;
   forming, using the healthcare data system computing device, a digital twin of said individual patient based on the health information related to said individual patient, the digital twin of said individual patient being a digital representation of at least one health state of said individual patient;
   forming, using the healthcare data system computing device, a digital twin of said population of patients based on the health information related to said population of patients, the digital twin of said population of patients being a digital representation of at least one health attribute of said population of patients; and
   presenting, at the healthcare data system computing device, to a user of the healthcare data system the digital twin of said patient and the digital twin of said population of patients.
2. The computerized method of healthcare data management of clause 1, further comprising:
   outputting, at the healthcare data system computing device, the digital twin of said patient and the digital twin of said population of patients to a machine learning module of the healthcare data system;
   simulating, at the healthcare data system computing device, a future health state of said patient based on the digital twin of said patient using the digital twin of said patient and the machine learning module;
   simulating, at the healthcare data system computing device, a future health state of said population of patients based on the digital twin of said population of patients using the digital twin of said population of patients and the machine learning module;
   updating, at the healthcare data system computing device, the digital twin of said patient based on the simulation of the future health state of said patient;
   updating, at the healthcare data system computing device, the digital twin of said population of patients based on the simulation of the future health state of said population of patients; and
   presenting, at the healthcare data system computing device, to said user of the healthcare data system the updated digital twin of said patient and the updated digital twin of said population of patients.
3. The computerized method of clause 2, wherein simulation of the future health state of said first population of patients and/or the future health state of said second population of patients is performed according to simulation instructions received from one or more of said healthcare workers.
4. The computerized method of clause 2, wherein simulation of the future health state of said first population of patients and/or the future health state of said second population of patients is performed according to simulation instructions formed by the machine learning module.
5. The computerized method of healthcare data management of clause 1, further comprising presenting, at the healthcare data system computing device, to said user of the healthcare data system the digital twins via a graphical interface including one or more of graphs, charts, and diagrams indicative of the health state of said patient.
6. The computerized method of healthcare data management of clause 5, further comprising comparing, using the healthcare data system computing device, the digital twin of said patient to the digital twin of said population of patients using the machine learning module, wherein the digital twin of said patient and/or one or more metrics thereof are presented to said user in comparison to the digital twin of one of said population of patients and one or more metrics of said population of patients.
7. The computerized method of clause 6, wherein the one or more metrics includes metrics related to health of said patient, health of said population of patients, and ideal disease state data, the ideal disease state data being based on best clinician standards and/or health outcomes.
8. The computerized method of healthcare data management of clause 2, further comprising determining, using the healthcare data system computing device, members of said population of patients disposed to developing one or more health issues using the machine learning module based on the simulation of the health state of said population of patients.
9. The computerized method of healthcare data management of clause 1, wherein health information received at the healthcare data system computing device includes an entire medical record of said patient.
10. The computerized method of healthcare data management of clause 2, further comprising simulating, using the healthcare data system computing device, effects of one or more healthcare treatment options for said patient and determining one or more corresponding potential future health states of said patient based on the digital twin of said patient using the digital twin of said patient and the machine learning module.
11. The computerized method of healthcare data management of clause 1, wherein the received health information includes lifestyle information related to exercise habits and diet of said patient and further comprising determining, using the healthcare data system computing device, effects of said exercise habits and diet of said patient on the health state of said patient based on the digital twin of said patient using the digital twin of said patient and the machine learning module.
12. The computerized method of healthcare data management of clause 1, wherein the lifestyle information includes one or more social determinants of health, the social determinants of health being related to one or more effects of lifestyle information on one or more health states of said patient and/or said population of patients.
13. The computerized method of clause 1, further comprising determining, using the healthcare data system computing device and the machine learning module, similar members of said population of patients having one or more similarities to said patient, the one or more similarities including one or more similarities in lifestyle, one or more similarities in on of diagnosis and prognosis, and one or more similarities in present or previous healthcare treatments.
14. The computerized method of healthcare data management of clause 13, further comprising identifying, using the healthcare data system computing device, one or more clinicians suited to treat said patient and said similar members based on one of specialization and experience of said clinicians in treating patients having said similarities.
15. The computerized method of clause 13, further comprising:
   receiving, at the healthcare data system computing device, continuous health information from one or more healthcare communication sources, the continuous health information including data related to an individual patient and data related to a population of patients, wherein the data related to said individual patient and the data related to said population of patients are related to one of lifestyle, diagnosis, prognosis, present healthcare treatments and previous healthcare treatments;
   updating, at the healthcare data system computing device, the digital twin of one of said patient and the digital twin of said population of patients based on the continuous health information;
   analyzing, using the healthcare data system computing device, effects of one of the lifestyle, the diagnosis, the prognosis, the present healthcare treatments, appropriate healthcare treatments leading to desired clinical outcome, and the previous healthcare treatments on said patient or said population of patients using the machine learning module and the one of digital twin of said patient and the digital twin of said population of patients.
16. The computerized method of clause 15, further comprising: classifying, at the healthcare data system computing device, the effects of the lifestyle, an economic class of said patient, diagnosis and/or prognosis, and/or present or previous healthcare treatments on said patient and/or said population of patients.
17. The computerized method of clause 1, further comprising:
   receiving, at the healthcare data system computing device, healthcare research information derived from a plurality of healthcare research sources; and
   correlating, using the healthcare data system computing device, the healthcare research information with the health information using the machine learning module to determine one of a relative accuracy of the healthcare research information and discrepancies between the healthcare research information and the health information.
18. The computerized method of clause 16, further comprising:
   receiving at the healthcare data computing device healthcare data entered by said patient; and
   correlating, using the healthcare data system computing device, the healthcare research information and the healthcare data entered by said patient with the health information using the machine learning module to determine one of a relative accuracy of the healthcare research information and discrepancies between the healthcare research information and the health information.
19. The computerized method of clause 15, further comprising performing, at the healthcare data system computing device, impact discovery analysis using the machine learning module to determine correlations between two or more sources of healthcare research information and/or the health information.
20. The computerized method of clause 1, wherein the health information further includes disease state information, the disease state information being indicative of one or more specific disease states of the patient and/or the population of patients.

### Set B—Exemplary Clauses

1. A computerized method for healthcare data management, the method comprising:
   receiving, at a healthcare data system computing device including one or more processors, health information from a plurality of healthcare communication sources, wherein the health information includes data related to an individual patient and data related to a population of patients;
   storing, using the healthcare data system computing device, the health information;
   correlating, using the healthcare data system computing device, the health information to one of said patient and said population of patients using the machine learning module using Bayesian graphical networks to determine patterns related to effects of one or more of lifestyle, diagnosis, prognosis, present healthcare treatments, and previous healthcare treatments based on of said patient and said population of patients;
   forming, at the healthcare data system computing device, a digital twin of said individual patient based on the health information related to said individual patient, wherein the digital twin of said individual patient is a digital representation of at least one health state of said individual patient;
   forming, at the healthcare data system computing device, a digital twin of said population of patients based on the health information related to said population of patients, wherein the digital twin of said population of patients is a digital representation of at least one health attribute of said population of patients; and
   presenting, at the healthcare data system computing device, to a user of the healthcare data system the digital twin of said patient with the health information of said patient, the digital twin of said population of patients with the health information of said population of patients, and data based on the correlating of the health information to one of said patient and said population of patients.
2. The computerized method of clause 1, further comprising categorizing, at the healthcare data system computing device, one or more patients according to one or more of lifestyle, diagnosis and/or prognosis, and present or previous healthcare treatments using the machine learning module, wherein the machine learning module applies fuzzy rules to categorize said one or more patients.
3. The computerized method of clause 2, wherein the healthcare data includes one or more social determinants of health and further comprising categorizing, at the healthcare data system computing device, the one or more social determinants of health using the machine learning module.
4. The computerized method of healthcare data management of clause 2, further comprising:
   outputting, at the healthcare data system computing device, the digital twin of said patient and the digital twin of said population of patients to a machine learning module of the healthcare data system;
   simulating, at the healthcare data system computing device, a future health state of said patient based on the digital twin of said patient using the digital twin of said patient and the machine learning module;
   simulating, at the healthcare data system computing device, a future health state of said population of patients based on the digital twin of said population of patients using the digital twin of said population of patients and the machine learning module;
   updating, at the healthcare data system computing device, the digital twin of said patient based on the simulation of the future health state of said patient;
   updating, at the healthcare data system computing device, the digital twin of said population of patients based on the simulation of the future health state of said population of patients; and
   presenting, at the healthcare data system computing device, to said user of the healthcare data system the updated digital twin of said patient and the updated digital twin of said population of patients;
   wherein said population of patients is determined based on one or more of lifestyle, diagnosis and/or prognosis, and present or previous healthcare treatments as categorized using the machine learning module.
5. The computerized method of clause 4, wherein simulation of the future health state of said first population of patients and/or the future health state of said second population of patients is performed according to simulation instructions received from one or more of said healthcare workers.
6. The computerized method of clause 4, wherein simulation of the future health state of said first population of patients and/or the future health state of said second population of patients is performed according to simulation instructions formed by the machine learning module.
7. The computerized method of clause 4, wherein said patient is a member of said population of patients and further comprising comparing the digital twin of said patient to the digital twin of said population of patients using the machine learning module.
8. The computerized method of clause 2, wherein the machine learning module applies at least one of a batch gradient descent and a stochastic gradient descent to categorize said one or more patients.
9. The computerized method of clause 1, further comprising comparing the digital twin of said individual patient with said health information to identify one or more gaps in care provided to said individual patient.
10. The computerized method of clause 9, wherein the one or more gaps in care include failure by one or more healthcare professionals to follow one or more established clinical standards of care in treating said patient.
11. The computerized method of clause 1, further comprising comparing the digital twin of said individual patient with said health information to identify one or more gaps in care provided to said population of patients.
12. The computerized method of clause 11, wherein the one or more gaps in care include failure by one or more healthcare professionals to follow one or more established clinical standards of care in treating said population of patients.

### Set C—Exemplary Clauses

1. A computerized method for healthcare data management, the method comprising:
   receiving, at a healthcare data system computing device including one or more processors, health information from a plurality of healthcare communication sources, the health information including data related to an individual patient and data related to a first population of patients;
   receiving, at the healthcare data system computing device, information indicative of a plurality of healthcare workers, each healthcare worker of said plurality of healthcare workers working together as a team to treat at least one of said individual patient, said first population of patients, and a second population of patients;
   forming, at the healthcare data system computing device, a digital twin of said individual patient based on the health information related to said individual patient, wherein the digital twin of said individual patient is a digital representation of at least one health state of said individual patient;
   forming, at the healthcare data system computing device, a digital twin of said population of patients based on the health information related to said population of patients, wherein the digital twin of said population of patients is a digital representation of at least one health attribute of said population of patients; and
   presenting, at the healthcare data system computing device, to each of the healthcare workers the digital twin of said individual patient and the digital twin of at least one of said first population of patients, second population of patients, and the health information.
2. The computerized method of clause 1, further comprising:
   receiving, at the healthcare data system computing device, healthcare research information derived from a plurality of healthcare research sources;
   determining, using the healthcare data system computing device and a machine learning module, whether at least a portion of the healthcare research information is relevant to at least one of said individual patient, said first population of patients, and said second population of patients; and
   presenting to each of the healthcare workers, at the healthcare data system computing device, the healthcare research information determined to be relevant to at least one of said individual patient, said first population of patients, and said second population of patients.
3. The computerized method of healthcare data management of clause 2, further comprising:
   outputting, at the healthcare data system computing device, the digital twin of said patient and the digital twin of said population of patients to a machine learning module of the healthcare data system;
   simulating, at the healthcare data system computing device, a future health state of said first population of patients based on the digital twin of said patient using the digital twin of said patient and the machine learning module;
   simulating, at the healthcare data system computing device, a future health state of said second population of patients based on the digital twin of said population of patients via the digital twin of said population of patients and the machine learning module;
   updating, at the healthcare data system computing device, the digital twin of said patient based on the simulation of the future health state of said patient;
   updating, at the healthcare data system computing device, the digital twin of said population of patients based on the simulation of the future health state of said population of patients; and
   presenting to each of the healthcare workers, at the healthcare data system computing device, the healthcare research information determined to be relevant to at least one of said individual patient, said first population of patients, and said second population of patients.
4. The computerized method of clause 3, wherein simulation of the future health state of said first population of patients and/or the future health state of said second population of patients is performed according to simulation instructions received from one or more of said healthcare workers.
5. The computerized method of clause 3, wherein simulation of the future health state of said first population of patients and/or the future health state of said second population of patients is performed according to simulation instructions formed by the machine learning module.
6. The computerized method of clause 1, further comprising: forming, using the healthcare data system computing device and a machine learning module, one or more models based on the health information related to at least one of a first and a second population of patients of said population of patients, wherein the one or models are configured to facilitate anticipating one or more responses to medical treatment by at least one of said first population of patients and said second population of patients.
7. The computerized method of clause 1, further comprising facilitating, using the healthcare data system computing device, opting into one or more treatment programs by at least one of said individual patient, a patient from said first population of patients, and a patient from said second population of patients.
8. The computerized method of clause 7, further comprising simulating, using the healthcare data system computing device and the machine learning module, effects of at least one of one or more drugs and treatment options on at least one of said individual patient, said first population of patients, and said second population of patients.
9. The computerized method of clause 8, further comprising comparing, using the healthcare data system computing device, simulations of one of one or more drugs and said treatment options to one or more of said treatment programs opted into by at least one of said individual patient, a patient from said first population of patients, and a patient from said second population of patients.
10. The computerized method of clause 9, further comprising:
   receiving, at the healthcare data system computing device, healthcare study information including at least one of methodology and results of one or more healthcare studies; and
   comparing, using the healthcare data system computing device and the machine learning module, the healthcare study information to the simulations of one or more said drugs and said treatment options to determine at least one of reliability and consistency of the simulations of one or more said drugs and treatment options.

### Set D—Exemplary Clauses

1. A computerized method for healthcare data management, the method comprising:
   receiving, at a healthcare data system computing device including one or more processors, health information from one or more healthcare communication sources, wherein the health information includes one or more of genetic, environmental, and lifestyle data related to an individual patient and one or more of genetic, environmental, and lifestyle data related to a population of patients;
   receiving, at the healthcare data system computing device, healthcare research information related to one or more of genetic, environmental, and lifestyle data;
   storing, using the healthcare data system computing device, the health information;
   forming, at the healthcare data system computing device, a digital twin of said individual patient based on the health information related to said individual patient, wherein the digital twin of said individual patient is a digital representation of at least one health state of said individual patient;
   forming, at the healthcare data system computing device, a digital twin of said population of patients based on the health information related to said population of patients, wherein the digital twin of said population of patients is a digital representation of at least one health attribute of said population of patients; and
   presenting, at the healthcare data system computing device, to a user of the healthcare data system the digital twin of said patient and the digital twin of said population of patients, the health information, and the healthcare research information.
2. The computerized method of clause 1, further comprising:
   receiving, at the healthcare data system computing device, desirability data from a healthcare researcher, wherein the desirability data is indicative of one or more of genetic, environmental, and lifestyle properties of a potential research subject desired by said healthcare researcher;
   comparing, at the healthcare data system computing device, the desirability data to the health information; and
   determining, at the healthcare data system computing device, whether one or more of said patient, said population of patients, and a subset of said population of patients are desired by said healthcare researcher based on the comparison of the desirability data to the health information.
3. The computerized method of clause 1, further comprising:
   comparing, at the healthcare data system computing device, the health information and the healthcare research information; and
   determining, at the healthcare data system computing device, similarities in one or more of genetic, environmental, and lifestyle data to related results of the healthcare research information.
4. The computerized method of clause 1, further comprising:
   receiving, at the healthcare data system computing device, sensor data from one or both of an environmental sensor and a wearable sensor worn by said patient;
   storing the sensor data using the healthcare data system computing device;
   processing the sensor data using the healthcare data system computing device and the machine learning module; and
   presenting the sensor data to a user of the healthcare data system using the healthcare data system computing device.
5. The computerized method of clause 4, further comprising:
   determining, using the healthcare data system computing device and the machine learning module, a personalized treatment plan for said patient based on at least one of the digital twin of said patient and the digital twin of said population of patients, the health information, the healthcare research information, and the sensor data; and
   presenting, at the healthcare data system computing device, the personalized treatment plan to a user of the healthcare data system.
6. The computerized method of clause 4, further comprising predicting, at the healthcare data system computing device, a response to a drug by said patient based on the genetic data of the health information using the machine learning module.
7. The computerized method of healthcare data management of clause 1, further comprising:
   outputting, at the healthcare data system computing device, the digital twin of said patient and the digital twin of said population of patients to a machine learning module of the healthcare data system;
   simulating, at the healthcare data system computing device, a future health state of said first population of patients based on the digital twin of said patient via the digital twin of said patient and the machine learning module;
   simulating, at the healthcare data system computing device, a future health state of said second population of patients based on the digital twin of said population of patients via the digital twin of said population of patients and the machine learning module;
   updating, at the healthcare data system computing device, the digital twin of said patient based on the simulation of the future health state of said patient;
   updating, at the healthcare data system computing device, the digital twin of said population of patients based on the simulation of the future health state of said population of patients; and
   presenting to each of the healthcare workers, at the healthcare data system computing device, the healthcare research information determined to be relevant to at least one of said individual patient, said first population of patients, and said second population of patients.
8. The computerized method of clause 7, further comprising:
   receiving, at the healthcare data system computing device, desirability data from a healthcare researcher, wherein the desirability data is indicative of one or more of genetic, environmental, and lifestyle properties of a potential research subject desired by said healthcare researcher;
   comparing, at the healthcare data system computing device, the desirability data to the health information; and
   determining, at the healthcare data system computing device, whether one or more of said patient, said population of patients, and a subset of said population of patients are desired by said healthcare researcher based on the comparison of the desirability data to the health information;
   wherein comparing the desirability data and determining whether one or more of said patient, said population of patients, and a subset of said population of patients are desired are performed based on said updated health information and/or said updated digital twins of said patient and/or said population of patients.
9. The computerized method of clause 7, further comprising:
   comparing, at the healthcare data system computing device, the health information and the healthcare research information; and
   determining, at the healthcare data system computing device, similarities in one or more of genetic, environmental, and lifestyle data to related results of the healthcare research information;
   wherein comparing the health information and the healthcare research information and determining similarities in one or more of genetic, environmental, and lifestyle data are performed based on said updated health information and/or said updated digital twins of said patient and/or said population of patients.
10. The computerized method of clause 7, wherein simulation of the future health state of said first population of patients and/or the future health state of said second population of patients is performed according to simulation instructions received from one or more of said healthcare workers.
11. The computerized method of clause 7, wherein simulation of the future health state of said first population of patients and/or the future health state of said second population of patients is performed according to simulation instructions formed by the machine learning module.

### Set E—Exemplary Clauses

1. A computerized method for healthcare data management, the method comprising:
   receiving, at a healthcare data system computing device including one or more processors, health information from one or more healthcare communication sources, wherein the health information includes data related to an individual patient and data related to a population of patients;
   storing the health information using the healthcare data system computing device;
   forming, at the healthcare data system computing device, a digital twin of said individual patient based on the health information related to said individual patient, wherein the digital twin of said individual patient is a digital representation of at least one health state of said individual patient;
   forming, at the healthcare data system computing device, a digital twin of said population of patients based on the health information related to said population of patients, wherein the digital twin of said population of patients is a digital representation of at least one health attribute of said population of patients;
   determining, at the healthcare data system computing device, whether said population of patients have one or more symptoms similar to said patient;
   presenting, at the healthcare data system computing device, to a user of the healthcare data system the digital twin of said patient, the digital twin of said population of patients, and the determination of whether said population of patients have one or more symptoms similar to said patient; and
   facilitating, at the healthcare data system computing device, consent for collaborative diagnosis of said patient and said population of patients by said user of the healthcare data system.
2. The computerized method of clause 1, further comprising:
   receiving, at the healthcare data system computing device, simulation instructions from a healthcare provider, the simulation instructions being indicative of one or more treatment plans;
   simulating, at the healthcare data system computing device, the one or more treatment plans, application of best clinical practices for a desired clinical outcome, and identification of any gaps in care on said patient via the digital twin of said patient; and
   evaluating, at the healthcare data system computing device, efficacy of the one or more treatment plans.
3. The computerized method of clause 2, further comprising simulating, at the healthcare data system computing device, application of best clinical practices for a desired clinical outcome on said patient via the digital twin of said patient.
4. The computerized method of clause 2, further comprising identifying any gaps in care provided to said patient using the digital twin of said patient.
5. The computerized method of clause 4, wherein the one or more gaps in care include failure by one or more healthcare professionals to follow one or more established clinical standards of care in treating said patient.
6. The computerized method of clause 1, further comprising:
   determining, at the healthcare data system computing device, one or more prognostication methods suitable for said population of patients using the machine learning module;
   simulating, at the healthcare data system computing device, the one or more prognostication methods on said population of patient via the digital twin of said population of patients; and
   evaluating, at the healthcare data system computing device, efficacy of the one or more prognostication methods.
7. The computerized method of clause 1, further comprising:
   receiving, at the healthcare data system computing device, simulation instructions from a healthcare researcher, the simulation instructions including one or more research experiments;
   simulating, at the healthcare data system computing device, the one or more research experiments, and results of best clinical practices on at least one of said patient and said population of patients using at least one of the digital twin of said patient and the digital twin of said population of patients.
8. The computerized method of clause 1, further comprising:
   receiving, at the healthcare data system computing device, simulation instructions from a healthcare researcher, the simulation instructions including one or more drug treatment regimens;
   simulating, at the healthcare data system computing device, the one or more drug treatment regimens on one or both of said patient and said population of patients v using at least one of the digital twin of said patient and the digital twin of said population of patients.
9. The computerized method of clause 1, further comprising:
   receiving, at the healthcare data system computing device, sensor data from one or more Internet of Things (IoT) sensors related to one or both of said patient and said population of patients; and
   updating at least one of the digital twin of said patient and the digital twin of said population of patients based on said population of patients based on the sensor data.
10. The computerized method of healthcare data management of clause 1, further comprising:
   outputting, at the healthcare data system computing device, the digital twin of said patient and the digital twin of said population of patients to a machine learning module of the healthcare data system;
   simulating, at the healthcare data system computing device, a future health state of said first population of patients based on the digital twin of said patient via the digital twin of said patient and the machine learning module;
   simulating, at the healthcare data system computing device, a future health state of said second population of patients based on the digital twin of said population of patients via the digital twin of said population of patients and the machine learning module;
   updating, at the healthcare data system computing device, the digital twin of said patient based on the simulation of the future health state of said patient;
   updating, at the healthcare data system computing device, the digital twin of said population of patients based on the simulation of the future health state of said population of patients; and
   presenting to each of the healthcare workers, at the healthcare data system computing device, the healthcare research information determined to be relevant to at least one of said individual patient, said first population of patients, and said second population of patients.
11. The computerized method of clause 10, wherein simulation of the future health state of said first population of patients and/or the future health state of said second population of patients is performed according to simulation instructions received from one or more of said healthcare workers.
12. The computerized method of clause 10, wherein simulation of the future health state of said first population of patients and/or the future health state of said second population of patients is performed according to simulation instructions formed by the machine learning module.

### Set F—Exemplary Clauses

1. A computerized method for healthcare data management, the method comprising:
   receiving, at a healthcare data system computing device including one or more processors, health information from one or more healthcare communication sources, wherein the health information including data related to an individual patient and data related to a population of patients;
   storing the health information using the healthcare data system computing device;
   forming, at the healthcare data system computing device, a digital twin of said individual patient based on the health information related to said individual patient, wherein the digital twin of said individual patient is a digital representation of at least one health state of said individual patient;
   forming, at the healthcare data system computing device, a digital twin of said population of patients based on the health information related to said population of patients, wherein the digital twin of said population of patients is a digital representation of at least one health attribute of said population of patients;
   presenting to a user of the healthcare data system, at the healthcare data system computing device, the digital twin of said patient and the digital twin of said population of patients;
   receiving, at the healthcare data system computing device, updated health information from one or more of said healthcare communication sources, and the updated health information including data related to said patient and data related to said population of patients collected by said healthcare communication sources subsequent to the healthcare data;
   updating, at the healthcare data system computing device, the digital twin of said patient and the digital twin of said population of patients based on the updated health information; and
   presenting to said user of the healthcare data system, at the healthcare data system computing device, the updated digital twins of said patient and said population of patients.
2. The computerized method of clause 1, wherein the health information includes data related to one or more interactions with healthcare providers by said patient.
3. The computerized method of clause 1, wherein the health information includes data related to compliance with one or more treatment plans by said patient, said one or more treatment plans having been prescribed by one or more healthcare providers.
4. The computerized method of clause 1, wherein the health information includes data related to one or more interactions with healthcare providers by said population of patients.
5. The computerized method of clause 1, wherein the health information includes data related to compliance with one or more treatment plans by said population of patients, said one or more treatment plans having been prescribed by one or more healthcare providers.
6. The computerized method of clause 1, further comprising:
   tracking, using the healthcare data system computing database and a machine learning module, changes in health of one or both of said patient and said population of patients based on the healthcare information and the updated healthcare information; and
   identifying, using the healthcare data system computing database and the machine learning module, indicators of potential future illness in one of said patient and said population of patients based on the tracked changes in health.
7. The computerized method of clause 6, wherein tracking changes in health is performed based on one or both of data related to one or more interactions with healthcare providers by said patient and data related to compliance with one or more treatment plans by said patient, said one or more treatment plans having been prescribed by one or more healthcare providers.
8. The computerized method of clause 6, wherein tracking changes in health is performed based on one or both of data related to one or more interactions with healthcare providers by said population of patients and data related to compliance with one or more treatment plans by said population of patients, said one or more treatment plans having been prescribed by one or more healthcare providers.
9. The computerized method of healthcare data management of clause 1, further comprising:
   outputting, at the healthcare data system computing device, the digital twin of said patient and the digital twin of said population of patients to a machine learning module of the healthcare data system;
   simulating, at the healthcare data system computing device, a future health state of said first population of patients based on the digital twin of said patient via the digital twin of said patient and the machine learning module;
   simulating, at the healthcare data system computing device, a future health state of said second population of patients based on the digital twin of said population of patients via the digital twin of said population of patients and the machine learning module;
   updating, at the healthcare data system computing device, the digital twin of said patient based on the simulation of the future health state of said patient;
   updating, at the healthcare data system computing device, the digital twin of said population of patients based on the simulation of the future health state of said population of patients; and
   presenting to each of the healthcare workers, at the healthcare data system computing device, the healthcare research information determined to be relevant to at least one of said individual patient, said first population of patients, and said second population of patients.
10. The computerized method of clause 10, wherein simulation of the future health state of said first population of patients and/or the future health state of said second population of patients is performed according to simulation instructions received from one or more of said healthcare workers.
11. The computerized method of clause 10, wherein simulation of the future health state of said first population of patients and/or the future health state of said second population of patients is performed according to simulation instructions formed by the machine learning module.

### Set G—Exemplary Clauses

1. A computerized method for healthcare data management, the method comprising:
   receiving, at a healthcare data system computing device including one or more processors, health information from one or more healthcare communication sources, wherein the health information includes data related to an individual patient and data related to a population of patients and includes data related to treatment provided to one or both of said patient and said population of patients;
   receiving, at the healthcare data system computing device, investment data related to money spent on one or both of research programs and treatment regimens by one or more of a healthcare provider, a healthcare researcher, and a health insurance provider;
   storing the health information using the healthcare data system computing device;
   forming, at the healthcare data system computing device, a digital twin of said individual patient based on the health information related to said individual patient, wherein the digital twin of said individual patient is a digital representation of at least one health state of said individual patient;
   forming, at the healthcare data system computing device, a digital twin of said population of patients based on the health information related to said population of patients, wherein the digital twin of said population of patients is a digital representation of at least one health attribute of said population of patients;
   presenting to a user of the healthcare data system, at the healthcare data system computing device, the digital twin of said patient and the digital twin of said population of patients;
   determining, using the healthcare data system computing device, a return on investment metric indicative of an amount of money invested versus an amount of money recovered by one or more of said healthcare provider, said healthcare researcher, and said health insurance provider based on said health information, said investment data, and one or both of the digital twins of said patient and said population of patients.
2. The computerized method of clause 1, further comprising receiving, at the healthcare data system computing device, investment data related to costs of care by one or more of the said healthcare provider, said healthcare researcher, and said health insurance provider.
3. The computerized method of clause 2, further comprising determining, using the healthcare data system computing device, the return on investment metric, wherein the return on investment metric is at least partially based on costs of care provided by one or more of said healthcare researcher, and said health insurance provider based on said health information, said investment data, and one or both of the digital twins of said patient and said population of patients.
4. The computerized method of clause 1, further comprising determining, using the healthcare data system computing device and a machine learning module, whether providing a first treatment to said patient and/or said population of patients rather than providing a second treatment to said patient and/or said population of patients may result in an improved return on investment metric.
5. The computerized method of clause 1, further comprising determining, using the healthcare data system computing device, an effect of a pre-existing condition on the return on investment metric of one of said patient and said population of patients.
6. The computerized method of healthcare data management of clause 1, further comprising:
   outputting, at the healthcare data system computing device, the digital twin of said patient and the digital twin of said population of patients to a machine learning module of the healthcare data system;
   simulating, at the healthcare data system computing device, a future health state of said first population of patients based on the digital twin of said patient via the digital twin of said patient and the machine learning module;
   simulating, at the healthcare data system computing device, a future health state of said second population of patients based on the digital twin of said population of patients via the digital twin of said population of patients and the machine learning module;
   updating, at the healthcare data system computing device, the digital twin of said patient based on the simulation of the future health state of said patient;
   updating, at the healthcare data system computing device, the digital twin of said population of patients based on the simulation of the future health state of said population of patients; and
   presenting to each of the healthcare workers, at the healthcare data system computing device, the healthcare research information determined to be relevant to at least one of said individual patient, said first population of patients, and said second population of patients.
7. The computerized method of clause 6, wherein simulation of the future health state of said first population of patients and/or the future health state of said second population of patients is performed according to simulation instructions received from one or more of said healthcare workers.
8. The computerized method of clause 6, wherein simulation of the future health state of said first population of patients and/or the future health state of said second population of patients is performed according to simulation instructions formed by the machine learning module.
9. The computerized method of clause 6, wherein the simulated digital twin of said patient is formed at least partially based on said investment data and includes a simulated return on investment metric.
10. The computerized method of clause 6, wherein the simulated digital twin of said population of patients is formed at least partially based on said investment data and includes a simulated return on investment metric.

### Set H—Exemplary Clauses

1. A system for characterizing the activities of one or more physicians in a health care drug prescription system, comprising:
   an interception module for retrieving PDMP information relating to the physicians;
   an interaction module identifying each sales and service representatives with whom the one or more physicians have interacted;
   an ordering module identifying orders from each of the organizations by the one or more physicians; and
   a correlation module that ensures that the PDMP information, the representatives with whom the physician has interacted and the orders are associated with correct records for the one or more physicians.
2. The system of clause 1, further comprising an insurance module that collects information from insurance records related to the one or more physicians.
3. The system of clause 1, further comprising a hospital module that collects information from hospital records related to the one or more physicians.
4. The system of clause 1, further comprising an analytics module that determines whether lab ordering patterns of the physicians and indicates whether a subset of the ordering patterns is anomalous.
5. The system of clause 4, wherein the analytics module determined whether lab ordering patterns of the physicians are indicative of over utilization and/or appropriate utilization of lab resources based on best practices and/or clinical guidelines.
6. A system for characterizing the activities of one or more patients in a health care system, comprising:
   an interception module for retrieving PDMP information relating to the one or more patients;
   a correlation module that ensures that the PDMP information is associated with the correct records of the one or more patients, and of the tests an analytics module that determines whether lab ordering patterns for the one or more patients and indicates whether a subset of the ordering patterns is anomalous.
7. The system of clause 6, further comprising a waste module that determines whether the one or more patients have taken one of unnecessary and redundant tests.
8. The system of clause 6, further comprising a prediction module that analyzes tests taken by the one or more patients results of the tests, and comparisons with aggregate information, and recommends additional tests for the one or more patients in order to detect additional conditions.
9. A method for analyzing the quality or effectiveness of a laboratory the method comprising: aggregating transaction information about a plurality of laboratories over time;
   analyzing volume and type of test from the transaction information;
   compiling a set of signals relating to pre-analytical, analytical, and post-analytical issues determined from the transaction information;
   parsing human-input information relating each of the issues determined from the transaction information;
   combining differently worded descriptions that are determined to have the same meaning; and automatically generating plain-language textual summaries that include at least a portion of detail from the issues determined from the transactional information.
10. The method of clause 9, wherein the plain-language textual summaries include one or more details of the issues with a particular laboratory from the plurality of laboratories.
11. The method of clause 9, wherein the plain-language textual summaries include an improvement plan and gaps in care report for a particular laboratory from the plurality of laboratories.
12. The method of clause 9, wherein mapping the issues determined from the transaction information to an ontology entity module containing descriptions of medical entities and automatically generating an indication of a most likely entity of the medical entities whose actions was a cause of the one or more issues.
13. A method for analyzing the quality or effectiveness of a laboratory, the method comprising:
   aggregating transaction information about a plurality of laboratories over time; analyzing volume and type of test from the transaction information;
   compiling a set of signals relating to one of test issues, speed, turnaround time, performance, and personnel determined from the transaction information; and
   compiling time utilization and workload statistics for each laboratory and each of its lab workers from the plurality of laboratories.
14. The method of clause 13, further comprising activating a workflow to identify one or more sources related to the set of signals that preceded a drop in productivity; and automatically activating a quality review of the one or more sources.
15. The method of clause 13, further comprising activating workflow to identify equipment related to the set of signals and automatically initiates a quality review of the equipment.

### Set I—Exemplary Clauses

1. A computerized method for healthcare data management, the method comprising:
   receiving, at a healthcare data system computing device including one or more processors, health information from one or more healthcare communication sources, the health information including data related to an individual patient and data related to a population of patients;
   storing, using the healthcare data system computing device, the health information;
   forming, using the healthcare data system computing device, a digital twin of said individual patient based on the health information related to said individual patient, the digital twin of said individual patient being a digital representation of at least one health state of said individual patient;
   forming, using the healthcare data system computing device, a digital twin of said population of patients based on the health information related to said population of patients, the digital twin of said population of patients being a digital representation of at least one health attribute of said population of patients; and
   presenting, at the healthcare data system computing device, to a user of the healthcare data system the digital twin of said patient and the digital twin of said population of patients.
2. The computerized method of clause 1, further comprising:
   outputting, at the healthcare data system computing device, the digital twin of said patient and the digital twin of said population of patients to a machine learning module of the healthcare data system;
   simulating, at the healthcare data system computing device, a future health state of said patient based on the digital twin of said patient via the digital twin of said patient and the machine learning module;
   simulating, at the healthcare data system computing device, a future health state of said population of patients based on the digital twin of said population of patients via the digital twin of said population of patients and the machine learning module;
   updating, at the healthcare data system computing device, the digital twin of said patient based on the simulation of the future health state of said patient;
   updating, at the healthcare data system computing device, the digital twin of said population of patients based on the simulation of the future health state of said population of patients; and
   presenting, at the healthcare data system computing device, to said user of the healthcare data system the updated digital twin of said patient and the updated digital twin of said population of patients.
3. The computerized method of clause 2, wherein simulation of the future health state of said first population of patients and/or the future health state of said second population of patients is performed according to simulation instructions received from one or more of said healthcare workers.
4. The computerized method of clause 2, wherein simulation of the future health state of said first population of patients and/or the future health state of said second population of patients is performed according to simulation instructions formed by the machine learning module.
5. The computerized method of clause 1, further comprising presenting, at the healthcare data system computing device, to said user of the healthcare data system the digital twins via a graphical interface including one or more of graphs, charts, and diagrams indicative of the health state of said patient.
6. The computerized method of clause 5, further comprising comparing, using the healthcare data system computing device, the digital twin of said patient to the digital twin of said population of patients using the machine learning module, wherein the digital twin of said patient and/or one or more metrics thereof are presented to said user in comparison to the digital twin of one of said population of patients and one or more metrics of said population of patients.
7. The computerized method of clause 6, wherein the one or more metrics includes metrics related to health of said patient, health of said population of patients, and ideal disease state data, the ideal disease state data being based on best clinician standards and/or health outcomes.
8. The computerized method of clause 2, further comprising determining, using the healthcare data system computing device, members of said population of patients disposed to developing one or more health issues using the machine learning module based on the simulation of the health state of said population of patients.
9. The computerized method of clause 1, wherein health information received at the healthcare data system computing device includes an entire medical record of said patient.
10. The computerized method of clause 2, further comprising simulating, using the healthcare data system computing device, effects of one or more healthcare treatment options for said patient and determining one or more corresponding potential future health states of said patient based on the digital twin of said patient via the digital twin of said patient and the machine learning module.
11. The computerized method of clause 1, wherein the received health information includes lifestyle information related to exercise habits and diet of said patient and further comprising determining, using the healthcare data system computing device, effects of said exercise habits and diet of said patient on the health state of said patient based on the digital twin of said patient using the digital twin of said patient and the machine learning module.
12. The computerized method of clause 1, wherein the lifestyle information includes one or more social determinants of health, the social determinants of health being related to one or more effects of lifestyle information on one or more health states of said patient and/or said population of patients.
13. The computerized method of clause 1, further comprising determining, using the healthcare data system computing device and the machine learning module, similar members of said population of patients having one or more similarities to said patient, the one or more similarities including one or more similarities in lifestyle, one or more similarities in on of diagnosis and prognosis, and one or more similarities in present or previous healthcare treatments.
14. The computerized method of clause 13, further comprising identifying, using the healthcare data system computing device, one or more clinicians suited to treat said patient and said similar members based on one of specialization and experience of said clinicians in treating patients having said similarities.
15. The computerized method of clause 13, further comprising:
   receiving, at the healthcare data system computing device, continuous health information from one or more healthcare communication sources, the continuous health information including data related to an individual patient and data related to a population of patients, wherein the data related to said individual patient and the data related to said population of patients are related to one of lifestyle, diagnosis, prognosis, present healthcare treatments and previous healthcare treatments;
   updating, at the healthcare data system computing device, the digital twin of one of said patient and the digital twin of said population of patients based on the continuous health information;
   analyzing, using the healthcare data system computing device, effects of one of the lifestyle, the diagnosis, the prognosis, the present healthcare treatments, appropriate healthcare treatments leading to desired clinical outcome, and the previous healthcare treatments on said patient or said population of patients using the machine learning module and the one of digital twin of said patient and the digital twin of said population of patients.
16. The computerized method of clause 15, further comprising: classifying, at the healthcare data system computing device, the effects of the lifestyle, an economic class of said patient, diagnosis and/or prognosis, and/or present or previous healthcare treatments on said patient and/or said population of patients.
17. The computerized method of clause 1, further comprising:
   receiving, at the healthcare data system computing device, healthcare research information derived from a plurality of healthcare research sources; and
   correlating, using the healthcare data system computing device, the healthcare research information with the health information using the machine learning module to determine one of a relative accuracy of the healthcare research information and discrepancies between the healthcare research information and the health information.
18. The computerized method of clause 16, further comprising:
   receiving at the healthcare data computing device healthcare data entered by said patient; and
   correlating, using the healthcare data system computing device, the healthcare research information and the healthcare data entered by said patient with the health information using the machine learning module to determine one of a relative accuracy of the healthcare research information and discrepancies between the healthcare research information and the health information.
19. The computerized method of clause 15, further comprising performing, at the healthcare data system computing device, impact discovery analysis using the machine learning module to determine correlations between two or more sources of healthcare research information and/or the health information.
20. The computerized method of clause 1, wherein the health information further includes disease state information, the disease state information being indicative of one or more specific disease states of the patient and/or the population of patients.

What is claimed is:

1. A system for characterizing the activities of one or more patients in a health care system, comprising:
an interception module for retrieving prescription drug data relating to the one or more patients;
a correlation module that ensures that the prescription drug data is associated with the correct records of the one or more patients, and
an analytics module that determines whether prescription ordering patterns for the one or more patients and indicates whether a subset of the ordering patterns is anomalous as compared with a stored ordering criterion.

2. The system of claim 1, further comprising a waste module that determines whether the one or more patients have taken one of unnecessary and redundant tests.

3. The system of claim 1, further comprising a prediction module that analyzes tests taken by the one or more patients results of the tests, and comparisons with aggregate information, and recommends additional tests for the one or more patients in order to detect additional conditions.

4. The system of claim 1, further comprising a machine learning module that infers relationships among prescription orders.

5. The system of claim 1, further comprising an artificial intelligence module that simulates future relationships among prescription orders.

6. A computerized method for healthcare data management, the method comprising:
receiving health data from one or more healthcare communication sources, wherein the health information includes data related to an individual patient and data related to a population of patients;
forming a digital twin of said individual patient based on the health data related to said individual patient, wherein the digital twin of said individual patient is a digital representation of at least one health state of said individual patient;
forming a digital twin of said population of patients based on the health data related to said population of patients, wherein the digital twin of said population of patients is a digital representation of at least one health attribute of said population of patients;
determining whether said population of patients have one or more symptoms similar to said patient;
simulating, using a machine learning module, a future health state of the individual patient;
detecting a new health state of the individual patient based at least in part on new health data received; and
transmitting an alert to the at least one healthcare provider indicating a discrepancy between the simulated future health state and the new health state.

7. The method of claim 6, wherein the machine learning simulation includes pharmaceutical data to simulate a future health state contingent upon the patient following a specified treatment plan.

8. The method of claim 6, wherein the machine learning simulation includes treatment plan data to simulate a future health state contingent upon the patient receiving a stated medication.

9. The method of claim 6, wherein the machine learning simulation uses a digital twin of the patient.

10. The method of claim 6, wherein the machine learning simulation uses a plurality of digital twins of the patient.

11. The method of claim 6, wherein simulation of the new health state is based in part on a measured health state of a population of patients matched to the individual patient according to a criterion.

12. The method of claim 6, wherein simulation of the new health state is based in part on a simulated health state of a population of patients matched to the individual patient according to a criterion.

13. The method of claim 6, further comprising:
simulating, using the machine learning module, effects of at least one of one or more drug treatment options of said individual patient, wherein the drug treatment options vary by the timing of providing mediation to the individual patient.

14. The method of claim 6, further comprising:
simulating, using the machine learning module, effects of at least one of one or more drug treatment options of said individual patient, wherein the drug treatment options vary by dosage level of mediation to the individual patient.

15. The method of claim 6, further comprising:
receiving healthcare study information including at least one of methodology and results of one or more healthcare studies; and
comparing, using the machine learning module, the healthcare study information to simulations of one or more said drug treatment options to determine at least one of reliability and consistency of the simulations of one or more said drug treatment options.

16. The method of claim 9, further comprising:
simulating application of best clinical practices for a desired clinical outcome on said individual patient via the digital twin of said individual patient.

17. The method of claim 6, further comprising:
receiving simulation instructions, the simulation instructions including one or more research experiments;
simulating the one or more research experiments, and results of best clinical practices on at least one of said individual patient and a population of patients using at least one of the digital twin of said individual patient and the digital twin of said population of patients.

18. The method of claim 6, further comprising:
receiving simulation instructions, the simulation instructions including one or more drug treatment regimens;
simulating the one or more drug treatment regimens on one or both of said individual patient and a population of patients using at least one of the digital twin of said individual patient and the digital twin of said population of patients.

19. The method of claim 18, wherein simulation of said individual patient and/or said population of patients is performed according to simulation instructions received from one or more of healthcare workers.

20. The method of claim 18, herein simulation of said individual patient and/or said population of patients is performed according to simulation instructions formed by the machine learning module.
